SEQUENCE LISTING (6 sequences)

SEQ ID NO: 1 - DNA, 551 bp, Homo Sapiens
accccatccgctggctctcacccctcggagacgctcgcccgacagcatagtacttgccgcccagccacgcccgcgcgccagccaccatgctaggtaacaagcgactggggctgtccggactgaccctcgccctgtccctgctcgtgtgcctgggtgcgctggccgaggcgtacccctccaagccggacaacccgggcgaggacgcaccagcggaggacatggccagatactactcggcgctgcgacactacatcaacctcatcaccaggcagagatatggaaaacgatccagcccagagacactgatttcagacctcttgatgagagaaagcacagaaaatgttcccagaactcggcttgaagaccctgcaatgtggtgatgggaaatgagacttgctctctggccttttcctattttcagcccatatttcatcgtgtaaaacgagaatccacccatcctaccaatgcatgcagccactgtgctgaattctgcaatgttttcctttgtcatcattgtatatatgtgtgtttaaataaagtatcatgcattc

SEQ ID NO: 2 - DNA, 2624 bp, Homo sapiens
  misc_feature (1622)..(1624): n=unknown
attgttcagttcaagggaatgaagaattcagaataattttggtaaatggattccaatatcgggaataagaataagctgaacagttgacctgctttgaagaaacatactgtccatttgtctaaaataatctataacaaccaaaccaatcaaaatgaattcaacattattttcccaggttgaaaatcattcagtccactctaatttctcagagaagaatgcccagcttctggcttttgaaaatgatgattgtcatctgcccttggccatgatatttaccttagctcttgcttatggagctgtgatcattcttggtgtctctggaaacctggccttgatcataatcatcttgaaacaaaaggagatgagaaatgttaccaacatcctgattgtgaacctttccttctcagacttgcttgttgccatcatgtgtctcccctttacatttgtctacacattaatggaccactgggtctttggtgaggcgatgtgtaagttgaatccttttgtgcaatgtgtttcaatcactgtgtccattttctctctggttctcattgctgtggaacgacatcagctgataatcaaccctcgagggtggagaccaaataatagacatgcttatgtaggtattgctgtgatttgggtccttgctgtggcttcttctttgcctttcctgatctaccaagtaatgactgatgagccgttccaaaatgtaacacttgatgcgtacaaagacaaatacgtgtgctttgatcaatttccatcggactctcataggttgtcttataccactctcctcttggtgctgcagtattttggtccactttgttttatatttatttgctacttcaagatatatatacgcctaaaaaggagaaacaacatgatggacaagatgagagacaataagtacaggtccagtgaaaccaaaagaatcaatatcatgctgctctccattgtggtagcatttgcagtctgctggctccctcttaccatctttaacactgtgtttgattggaatcatcagatcattgctacctgcaaccacaatctgttattcctgctctgccacctcacagcaatgatatccacttgtgtcaaccccatattttatgggttcctgaacaaaaacttccagagagacttgcagttcttcttcaacttttgtgatttccggtctcgggatgatgattatgaaacaatagccatgtccacgatgcacacagatgtttccaaaacttctttgaagcaagcaagcccagtcgcatttaaaaaaatcaacaacaatgatgataatgaaaaaatctgaaactacttatagcctatggtcccggatgacatctgtttaaaaacaagcacaacctgcaacatactttgattacctgttctcccaaggaatggggttgaaatcatttgaaaatgactaagattttcttgtcttgcttttttactgcttttgttgtagtgtcataattacatttggaacaaaaggtgtgggctttggggtcttctggaaatagttttgaccagacatctttgaagtgctttttgtgaatttatgcatataatataaagacttttatactgtacttattggaatgaaatttctttaaagtattacgatnnnctgacttcagaagtacctgccatccaatacggtcattagattgggtcatcttgattagattagattagattagattgtcaacagattgggccatccttactttatgataggcatcattttagtgtgttacaatagtaacagtatgcaaaagcagcattcaggagccgaaagatagtcttgaagtcattcagaagtggtttgaggtttctgttttttggtggtttttgtttgttttttttttttttcaccttaagggaggctttcatttcctcccgactgattgtcacttaaatcaaaatttaaaaatgaataaaaagacatacttctcagctgcaaatattatggagaattgggcacccacaggaatgaagagagaaagcagctccccaacttcaaaaccattttggtacctgacaacaagagcattttagagtaattaatttaataaagtaaattagtattgctgcaaatagctaaattatatttatttgaattgatggtcaagagattttccattttttttacagactgttcagtgtttgtcaagcttctggtctaatatgtactcgaaagactttccgcttacaatttgtagaaacacaaatatcgttttccatacagcagtgcctatatagtgactgattttaactttcaatgtccatctttcaaaggaagtaacaccaaggtacaatgttaaaggaatattcactttacctagcagggaaaaatacacaaaaactgcagatacttcatatagcccattttaacttgtataaactgtgtgacttgtggcgtcttataaataatgcactgtaaagattactgaatagttgtgtcatgttaatgtgcctaatttcatgtatcttgtaatcatgattgagcctcagaatcatttggagaaactatattttaaagaacaagacatacttcaatgtattatacagataaagtattacatgtgtttgattttaaaagggcggacattttattaaaatcaagg

SEQ ID NO: 3 - DNA, 1200 bp, Homo sapiens
caagtggacctgtactgaaaatgggtccaataggtgcagaggctgatgagaaccagacagtggaagaaatgaaggtggaacaatacgggccacaaacaactcctagaggtgaactggtccctgaccctgagccagagcttatagatagtaccaagctgattgaggtacaagttgttctcatattggcctactgctccatcatcttgcttggggtaattggcaactccttggtgatccatgtggtgatcaaattcaagagcatgcgcacagtaaccaactttttcattgccaatctggctgtggcagatcttttggtgaacactctgtgtctaccgttcactcttacctataccttaatgggggagtggaaaatgggtcctgtcctgtgccacctggtgccctatgcccagggcctggcagtacaagtatccacaatcaccttgacagtaattgccctggaccggcacaggtgcatcgtctaccacctagagagcaagatctccaagcgaatcagcttcctgattattggcttggcctggggcatcagtgccctgctggcaagtcccctggccatcttccgggagtattcgctgattgagatcatcccggactttgagattgtggcctgtactgaaaagtggcctggcgaggagaagagcatctatggcactgtctatagtctttcttccttgttgatcttgtatgttttgcctctgggcattatatcattttcctacactcgcatttggagtaaattgaagaaccatgtcagtcctggagctgcaaatgaccactaccatcagcgaaggcaaaaaaccaccaaaatgctggtgtgtgtggtggtggtgtttgcggtcagctggctgcctctccatgccttccagcttgccgttgacattgacagccaggtcctggacctgaaggagtacaaactcatcttcacagtgttccacatcatcgccatgtgctccacttttgccaatccccttctctatggctggatgaacagcaactacagaaaggctttcctctcggccttccgctgtgagcagcggttggatgccattcactctgaggtgtccgtgacattcaaggctaaaaagaacctggaggtcagaaagaacagtggccccaatgactctttcacagaggctaccaatgtctaaggaagctgtggtgtgaaaatgtatggatgaattc

SEQ ID NO: 4 - DNA, 2893 bp, Homo sapiens
ggcaccagctcagccccaagccactgctctcccatcccagtccctggaaatccacccacttggcccagctcaccccaactccaacccactgggacccagtctccaggggcctgactgtgggcggcagccactcctgagtgagcaaaggttcctccgcggtgctctcccgtccagagccctgctgatggggaagtccgaaggccccgtggggatggtggagagcgctggccgtgcagggcagaagcgcccggggttcctggagggggggctgctgctgctgctgctgctggtgaccgctgccctggtggccttgggtgtcctctacgccgaccgcagagggaagcagctgccacgccttgctagccggctgtgcttcttacaggaggagaggacctttgtaaaacgaaaaccccgagggatcccagaggcccaagaggtgagcgaggtctgcaccacccctggctgcgtgatagcagctgccaggatcctccagaacatggacccgaccacggaaccgtgtgacgacttctaccagtttgcatgcggaggctggctgcggcgccacgtgatccctgagaccaactcaagatacagcatctttgacgtcctccgcgacgagctggaggtcatcctcaaagcggtgctggagaattcgactgccaaggaccggccggctgtggagaaggccaggacgctgtaccgctcctgcatgaaccagagtgtgatagagaagcgaggctctcagcccctgctggacatcttggaggtggtgggaggctggccggtggcgatggacaggtggaacgagaccgtaggactcgagtgggagctggagcggcagctggcgctgatgaactcacagttcaacaggcgcgtcctcatcgacctcttcatctggaacgacgaccagaactccagccggcacatcatctacatagaccagcccaccttgggcatgccctcccgagagtactacttcaacggcggcagcaaccggaaggtgcgggaagcctacctgcagttcatggtgtcagtggccacgttgctgcgggaggatgcaaacctgcccagggacagctgcctggtgcaggaggacatggtgcaggtgctggagctggagacacagctggccaaggccacggtaccccaggaggagagacacgacgtcatcgccttgtaccaccggatgggactggaggagctgcaaagccagtttggcctgaagggatttaactggactctgttcatacaaactgtgctatcctctgtcaaaatcaagctgctgccagatgaggaagtggtggtctatggcatcccctacctgcagaaccttgaaaacatcatcgacacctactcagccaggaccatacagaactacctggtctggcgcctggtgctggaccgcattggtagcctaagccagagattcaaggacacacgagtgaactaccgcaaggcgctgtttggcacaatggtggaggaggtgcgctggcgtgaatgtgtgggctacgtcaacagcaacatggagaacgccgtgggctccctctacgtcagggaggcgttccctggagacagcaagagcatggtcagagaactcattgacaaggtgcggacagtgtttgtggagacgctggacgagctgggctggatggacgaggagtccaagaagaaggcgcaggagaaggccatgagcatccgggagcagatcgggcaccctgactacatcctggaggagatgaacaggcgcctggacgaggagtactccaatctgaacttctcagaggacctgtactttgagaacagtctgcagaacctcaaggtgggcgcccagcggagcctcaggaagcttcgggaaaaggtggacccaaatctctggatcatcggggcggcggtggtcaatgcgttctactccccaaaccgaaaccagattgtattccctgccgggatcctccagccccccttcttcagcaaggagcagccacaggccttgaactttggaggcattgggatggtgatcgggcacgagatcacgcacggctttgacgacaatggccggaacttcgacaagaatggcaacatgatggattggtggagtaacttctccacccagcacttccgggagcagtcagagtgcatgatctaccagtacggcaactactcctgggacctggcagacgaacagaacgtgaacggattcaacacccttggggaaaacattgctgacaacggaggggtgcggcaagcctataaggcctacctcaagtggatggcagagggtggcaaggaccagcagctgcccggcctggatctcacccatgagcagctcttcttcatcaactatgcccaggtgtggtgcgggtcctaccggcccgagttcgccatccaatccatcaagacagacgtccacagtcccctgaagtacagggtactggggtcgctgcagaacctggccgccttcgcagacacgttccactgtgcccggggcacccccatgcaccccaaggagcgatgccgcgtgtggtagccaaggccctgccgcgctgtgcggcccacgcccacctgctgctcggaggcatctgtgcgaaggtgcagctagcggcgacccagtgtacgtcccgccccggccaaccatgccaagcctgcctgccaggcctctgcgcctggcctagggtgcagccacctgcctgacacccagggatgagcagtgtccagtgcagtacctggaccggagccccctccacagacacccgcggggctcagtgcccccgtcacagctctgtagagacaatcaactgtgtcctgcccaccctccaaggtgcattgtcttccagtatctacagcttcagacttgagctaagtaaatgcttcaaagaaaaaaaaaaaaaaaaaaaaa

SEQ ID NO: 5 - DNA, 2975 bp, Homo sapiens
cagagctcgtttagtgaaccgtcagaattttgtaatacgactcactatagggcggccgcgaattcggcaccagctcagccccaagccactgctctcccatcccagtccctggaaatccacccacttggcccagctcaccccaactccaacccactgggacccagtctccaggggcctgactgtgggcggcagccactcctgagtgagcaaaggttcctccgcggtgctctcccgtccagagccctgctgatggggaagtccgaaggccccgtggggatggtggagagcgctggccgtgcagggcagaagcgcccggggttcctggagggggggctgctgctgctgctgctgctggtgaccgctgccctggtggccttgggtgtcctctacgccgaccgcagagggaagcagctgccacgccttgctagccggctgtgcttcttacaggaggagaggacctttgtaaaacgaaaaccccgagggatcccagaggcccaagaggtgagcgaggtctgcaccacccctggctgcgtgatagcagctgccaggatcctccagaacatggacccgaccacggaaccgtgtgacgacttctaccagtttgcatgcggaggctggctgcggcgccacgtgatccctgagaccaactcaagatacagcatctttgacgtcctccgcgacgagctggaggtcatcctcaaagcggtgctggagaattcgactgccaaggaccggccggctgtggagaaggccaggacgctgtaccgctcctgcatgaaccagagtgtgatagagaagcgaggctctcagcccctgctggacatcttggaggtggtgggaggctggccggtggcgatggacaggtggaacgagaccgtaggactcgagtgggagctggagcggcagctggcgctgatgaactcacagttcaacaggcgcgtcctcatcgacctcttcatctggaacgacgaccagaactccagccggcacatcatctacatagaccagcccaccttgggcatgccctcccgagagtactacttcaacggcggcagcaaccggaaggtgcgggaagcctacctgcagttcatggtgtcagtggccacgttgctgcgggaggatgcaaacctgcccagggacagctgcctggtgcaggaggacatggtgcaggtgctggagctggagacacagctggccaaggccacggtaccccaggaggagagacacgacgtcatcgccttgtaccaccggatgggactggaggagctgcaaagccagtttggcctgaagggatttaactggactctgttcatacaaactgtgctatcctctgtcaaaatcaagctgctgccagatgaggaagtggtggtctatggcatcccctacctgcagaaccttgaaaacatcatcgacacctactcagccaggaccatacagaactacctggtctggcgcctggtgctggaccgcattggtagcctaagccagagattcaaggacacacgagtgaactaccgcaaggcgctgtttggcacaatggtggaggaggtgcgctggcgtgaatgtgtgggctacgtcaacagcaacatggagaacgccgtgggctccctctacgtcagggaggcgttccctggagacagcaagagcatggtcagagaactcattgacaaggtgcggacagtgtttgtggagacgctggacgagctgggctggatggacgaggagtccaagaagaaggcgcaggagaaggccatgagcatccgggagcagatcgggcaccctgactacatcctggaggagatgaacaggcgcctggacgaggagtactccaatctgaacttctcagaggacctgtactttgagaacagtctgcagaacctcaaggtgggcgcccagcggagcctcaggaagcttcgggaaaaggtggacccaaatctctggatcatcggggcggcggtggtcaatgcgttctactccccaaaccgaaaccagattgtattccctgccgggatcctccagccccccttcttcagcaaggagcagccacaggccttgaactttggaggcattgggatggtgatcgggcacgagatcacgcacggctttgacgacaatggccggaacttcgacaagaatggcaacatgatggattggtggagtaacttctccacccagcacttccgggagcagtcagagtgcatgatctaccagtacggcaactactcctgggacctggcagacgaacagaacgtgaacggattcaacacccttggggaaaacattgctgacaacggaggggtgcggcaagcctataaggcctacctcaagtggatggcagagggtggcaaggaccagcagctgcccggcctggatctcacccatgagcagctcttcttcatcaactatgcccaggtgtggtgcgggtcctaccggcccgagttcgccatccaatccatcaagacagacgtccacagtcccctgaagtacagggtactggggtcgctgcagaacctggccgccttcgcagacacgttccactgtgcccggggcacccccatgcaccccaaggagcgatgccgcgtgtggtagccaaggccctgccgcgctgtgcggcccacgcccacctgctgctcggaggcatctgtgcgaaggtgcagctagcggcgacccagtgtacgtcccgccccggccaaccatgccaagcctgcctgccaggcctctgcgcctggcctagggtgcagccacctgcctgacacccagggatgagcagtgtccagtgcagtacctggaccggagccccctccacagacacccgcggggctcagtgcccccgtcacagctctgtagagacaatcaactgtgtcctgcccaccctccaaggtgcattgtcttccagtatctacagcttcagacttgagctaagtaaatgcttcaaagaaaaaaaaaaaaaaaaaaaaactcgactctagattgcg

SEQ ID NO: 6 - protein, 779 aa, Homo sapiens
MGKSEGPVGMVESAGRAGQKRPGFLEGGLLLLLLLVTAALVALGVLYADRRGKQLPRLASRLCFLQEERTFVKRKPRGIPEAQEVSEVCTTPGCVIAAARILQNMDPTTEPCDDFYQFACGGWLRRHVIPETNSRYSIFDVLRDELEVILKAVLENSTAKDRPAVEKARTLYRSCMNQSVIEKRGSQPLLDILEVVGGWPVAMDRWNETVGLEWELERQLALMNSQFNRRVLIDLFIWNDDQNSSRHIIYIDQPTLGMPSREYYFNGGSNRKVREAYLQFMVSVATLLREDANLPRDSCLVQEDMVQVLELETQLAKATVPQEERHDVIALYHRMGLEELQSQFGLKGFNWTLFIQTVLSSVKIKLLPDEEVVVYGIPYLQNLENIIDTYSARTIQNYLVWRLVLDRIGSLSQRFKDTRVNYRKALFGTMVEEVRWRECVGYVNSNMENAVGSLYVREAFPGDSKSMVRELIDKVRTVFVETLDELGWMDEESKKKAQEKAMSIREQIGHPDYILEEMNRRLDEEYSNLNFSEDLYFENSLQNLKVGAQRSLRKLREKVDPNLWIIGAAVVNAFYSPNRNQIVFPAGILQPPFFSKEQPQALNFGGIGMVIGHEITHGFDDNGRNFDKNGNMMDWWSNFSTQHFREQSECMIYQYGNYSWDLADEQNVNGFNTLGENIADNGGVRQAYKAYLKWMAEGGKDQQLPGLDLTHEQLFFINYAQVWCGSYRPEFAIQSIKTDVHSPLKYRVLGSLQNLAAFADTFHCARGTPMHPKERCRVW

(19) United States
(12) Patent Application Publication
(10) Pub. No.: US 2003/0119714 A1
(43) Pub. Date: Jun. 26, 2003

(54) Treatment of male sexual dysfunction

(76) Inventors: Alasdair Mark Naylor, Sandwich (GB); Pieter Hadewijn Van Der Graaf, Sandwich (GB); Christopher Peter Wayman, Sandwich (GB)

Correspondence Address: Gregg C. Benson, Pfizer Inc., Patent Department, MS4159, Eastern Point Road, Groton, CT, 06340, US

(21) Appl. No.: 10/017,273

(22) Filed: Dec. 12, 2001

Related U.S. Application Data
(63) Continuation-in-part of application No. 09/895,367, filed on Jun. 29, 2001.
(63) Continuation-in-part of application No. 09/905,846, filed on Jul. 13, 2001.
(60) Provisional application No. 60/265,358, filed on Jan. 31, 2001.
(60) Provisional application No. 60/291,722, filed on May 17, 2001.

(30) Foreign Application Priority Data
Dec. 15, 2000 (GB) 0030647.2
Apr. 6, 2001 (GB) 0108730.3
Apr. 23, 2001 (GB) 0109910.0
Apr. 5, 2001 (GB) 0111037.8
Aug. 24, 2001 (GB) 0120679.6

(51) Int. Cl. A61K 31/00

(52) U.S. Cl. 514/1

(57) ABSTRACT

The use of an inhibitor of a neuropeptide Y (NPY), preferably of a NPY Y1 receptor, which inhibitor is selective for an NPY or NPY Y1 receptor associated with male genitalia, in the preparation/manufacture of a medicament for the treatment or prevention of male erectile dysfunction (MED).

### FIELD OF INVENTION

The present invention relates to a compound and a pharmaceutical that is useful for the treatment and/or prevention of male sexual dysfunction (MSD), in particular male erectile dysfunction (MED).

The present invention also relates to a method of prevention and/or treatment of MSD, in particular MED.

The present invention also relates to assays to screen for the compounds useful in the treatment of MSD, in particular MED.

For convenience, a list of abbreviations that are used in the following text is presented before the claims section.

### BACKGROUND TO THE INVENTION

Sexual dysfunction (SD) is a significant clinical problem which can affect both males and females. The causes of SD may be both organic as well as psychological. Organic aspects of SD are typically caused by underlying vascular diseases, such as those associated with hypertension or diabetes mellitus, by prescription medication and/or by psychiatric disease such as depression. Physiological factors include fear, performance anxiety and interpersonal conflict. SD impairs sexual performance, diminishes self-esteem and disrupts personal relationships thereby inducing personal distress. In the clinic, SD disorders have been divided into female sexual dysfunction (FSD) disorders and male sexual dysfunction (MSD) disorders (Melman et al 1999). FSD is best defined as the difficulty or inability of a woman to find satisfaction in sexual expression. Male sexual dysfunction (MSD) is generally associated with erectile dysfunction, also known as male erectile dysfunction (MED) (Benet et al 1994).

Male erectile dysfunction (MED), otherwise known as male erectile disorder, is defined as:

“the inability to achieve and/or maintain a penile erection for satisfactory sexual performance” (NIH Consensus Development Panel on Impotence, 1993)”

It has been estimated that the prevalence of erectile dysfunction (ED) of all degrees (minimal, moderate and complete impotence) is 52% in men 40 to 70 years old, with higher rates in those older than 70 (Melman et al 1999). The condition has a significant negative impact on the quality of life of the individual and their partner, often resulting in increased anxiety and tension which leads to depression and low self esteem. Whereas two decades ago, MED was primarily considered to be a psychological disorder (Benet et al 1994), it is now known that for the majority of individuals there is an underlying organic cause. As a result, much progress has been made in identifying the mechanism of normal penile erection and the pathophysiologies of MED.

Penile erection is a haemodynamic event which is dependent upon the balance of contraction and relaxation of the corpus cavernosal smooth muscle and vasculature of the penis (Lerner et al 1993). Corpus cavernosal smooth muscle is also referred to herein as corporal smooth muscle or in the plural sense corpus cavernosa. Relaxation of the corpus cavernosal smooth muscle leads to an increased blood flow into the trabecular spaces of the corpus cavernosa, causing them to expand against the surrounding tunica and compress the draining veins. This produces a vast elevation in blood pressure which results in an erection (Naylor, 1998).

The changes that occur during the erectile process are complex and require a high degree of coordinated control involving the peripheral and central nervous systems, and the endocrine system (Naylor, 1998). Corporal smooth muscle contraction is modulated by sympathetic noradrenergic innervation via activation of postsynaptic a, adrenoceptors. MED may be associated with an increase in the endogenous smooth muscle tone of the corpus cavernosum. However, the process of corporal smooth muscle relaxation is mediated partly by non-adrenergic, non-cholinergic (NANC) neurotransmission. There are a number of other NANC neurotransmitters found in the penis, other than NO, such as calcitonin gene related peptide (CGRP) and vasoactive intestinal peptide (VIP). The main relaxing factor responsible for mediating this relaxation is nitric oxide (NO), which is synthesised from L-arginine by nitric oxide synthase (NOS) (Taub et al 1993; Chuang et al 1998). It is thought that reducing corporal smooth muscle tone may aid NO to induce relaxation of the corpus cavernosum. During sexual arousal in the male, NO is released from neurones and the endothelium and binds to and activates soluble guanylate cyclase (sGC) located in the smooth muscle cells and endothelium, leading to an elevation in intracellular cyclic guanosine 3′,5′-monophosphate (cGMP) levels. This rise in cGMP leads to a relaxation of the corpus cavernosum due to a reduction in the intracellular calcium concentration ([Ca²⁺]ᵢ), via unknown mechanisms thought to involve protein kinase G activation (possibly due to activation of Ca²⁺ pumps and Ca²⁺-activated K⁺ channels; Chuang et al., 1998).

Sildenafil citrate (also known as Viagra™) has recently been developed by Pfizer as the first oral drug treatment for MED. Sildenafil acts by inhibiting cGMP breakdown in the corpus cavernosa by selectively inhibiting phosphodiesterase 5 (PDE5), thereby limiting the hydrolysis of cGMP to 5′GMP (Boolel et al., 1996; Jeremy et al., 1997) and thereby increasing the intracellular concentrations of cGMP and facilitating corpus cavernosal smooth muscle relaxation.

Currently, all other available MED therapies on the market, such as treatment with prostaglandin based compounds i.e. alprostadil which can be administered intra-urethrally (available from Vivus Inc., as Muse™) or via small needle injection (available from Pharamcia & Upjohn, as Caverject™), are either inconvenient and/or invasive. Other treatments include vacuum constriction devices, vasoactive drug injection or penile prostheses implantation (Montague et al., 1996). Although injectable vasoactive drugs show high efficacy, side effects such as penile pain, fibrosis and priapism are common, and injection therapy is not as convenient as oral therapy therefore sildenafil currently represents the most preferred therapy on the market.

Thus, it is desirable to find new ways of treating male sexual dysfunction, in particular MED.

### SUMMARY ASPECTS

A seminal finding of the present invention is the ability to selectively treat a male suffering from sexual dysfunction, in particular MED, with use of a neuropeptide Y inhibitor (NPYi), preferably a NPY Y1 receptor inhibitor (NPY Y1i), without peripheral side effects. Surprisingly the applicants have also found that inhibition of NPY, preferably NPY Y1, with a neuropeptide Y inhibitor, hereinafter referred to as a NPYi, significantly enhances the nerve-stimulated erectile process. The terms I:NPY and NPYi, and I:NPY Y1 and NPY Y1i, are used interchangeably hereinafter.

The term “without peripheral side effects” as used herein means that the NPYi, preferably NPY Y1i, is devoid, or substantially devoid, of any activity on the cardiovascular system. Thus, the NPYi, preferably NPY Y1i, is inactive in the cardiovascular system, thus reducing or eliminating the prospect of cardiovascular events, such as a drop in blood pressure when the NPYi, preferably NPY Y1i, is administered systemically (i.e. by mouth). Peripheral side effects are those resulting from the inhibition of NPY or NPY Y1 receptors other than those in the genitalia and/or central nervous system. The NPYi, preferably NPY Y1i, according to the present invention may, in addition to acting on NPY, preferably NPY Y1 receptors, in the genitalia, act centrally on the central nervous system to effectively treat for example abnormal drink and food intake disorders, such as obesity, anorexia, bulimia and metabolic disorders. However, the NPYi, preferably NPY Y1i, according to the present invention when in use preferably has no, or substantially no, peripheral activity, i.e. on the cardiovascular system and/or the gastrointestinal system, other than that in respect of the genitalia. Thus there is systemic selectivity of the genitalia, although some activity in the central nervous system may also occur.

According to the present invention there is provided the use of an inhibitor of a neuropeptide Y (NPY), preferably an inhibitor of a NPY Y1 receptor, which when in use is selective, or highly selective, for NPY or NPY Y1 receptors associated with male genitalia for the treatment of male sexual dysfunction, in particular MED.

According to the present invention there is provided the use of an inhibitor of a neuropeptide Y (NPY), preferably an inhibitor of a NPY Y1 receptor, which when in use is selective, or highly selective, for NPY or NPY Y1 receptors associated with male genitalia for enhancing nerve-stimulated erectile process.

Preferably, the NPY/NPY Y1inhibitors for use in the treatment of male sexual dysfunction, in particular MED according to the present invention have an IC₅₀ of less than 100 nanomolar (nM), preferably of less than 75 nM, more preferably of less than 50 nM.

The term “selective” as used herein means the NPY inhibitors according to the present invention have greater than about 100-fold, more preferably greater than about 300-fold selectivity for NPY, in particular NPY Y1 receptors, in male genitalia, preferably in the corpus cavernosum, over NPY Y2 or NPY Y5 receptors. Preferably the NPYi or NPY Y1i has no, or substantially no, activity towards endopeptidase NEP EC 3.4.24.11 and/or angiotensin converting enzyme (ACE). Preferably, the NPY or NPY Y1inhibitors according to the present invention have no activity towards endothelin converting enzyme (ECE). Suitably, the NPY or NPY Y1inhibitors according to the present invention have greater than 300-fold, more preferably greater than 500-fold, more preferably greater than 1000-fold selectivity for NPY/NPY Y1 over NEP and/or ACE. Preferably the NPYi also has a greater than 1000-fold selectivity over ECE. This reduces the prospect of cardiovascular events (e.g. drop in blood pressure) when the NPYi or NPY Y1i is administered systemically (e.g. by mouth). The term “selectively” should be construed accordingly.

The term “highly selective” as used herein means the NPY/NPY Y1inhibitors according to the present invention have greater than about 400-fold selectivity, preferably at least about 500-fold selectivity, preferably at least about 600-fold selectivity, preferably at least about 700-fold selectivity, preferably at least about 800-fold activity, preferably at least about 900-fold activity, preferably at least about 1000-fold selectivity for NPY, in particular NPY Y1 receptors, in male genitalia (particularly in the corpus cavernosum) over NPY Y2 or NPY Y5 receptors. Preferably the NPYi or NPY Y1i has no, or substantially no, activity towards NEP and/or ACE and/or ECE. The term “highly selectively” should be construed accordingly.

Preferably, the NPY inhibitors, preferably NPY Y1inhibitors, for use in the treatment of male sexual dysfunction, in particular MED, according to the present invention when in use are highly selective for the reproductive tract. Thus, the use of an NPY inhibitor, preferably an NPY Y1i, results in localised activity in the genitalia and/or no, or substantially no, activity in the cardiovascular system. This reduces the prospect of cardiovascular events (e.g. drop in blood pressure) when the NPYi, preferably NPY Y1i, is administered systemically (e.g. by mouth).

There is further provided the use of an NPYi, preferably an NPY Y1i, in the manufacture of a medicament for the selective treatment and/or selective prevention of MED.

There is further provided the use of an NPYi, preferably an NPY Y1i, in the preparation of a medicament for the selective treatment and/or prevention of MED. Here, the NPYi or the NPY Y1i may be used in, for example, a manufacturing step and/or an identification preparative step and/or a modification preparative step of an agent according to the present invention.

The reported function of NPY in the penis is its role in the venous occlusion mechanism that occurs at penile level to sustain erections. That is to say, it has been reported that NPY acts as a vasoconstrictor and causes restriction of penile veins, in particular those which regulate the flow of blood from the penis. Thus, during an erection NPY was thought to aid the maintenance of an erection by causing constriction of the penile veins, therefore, preventing or reducing the flow of blood from the corpus cavernosum in the penis. In light of the teachings prior to the present invention, inhibition of NPY would have been expected to result in the relaxation of the penile veins regulating the flow of blood from the corpus cavernosum and, thus, the administration of an NPYi or an NPY Y1i would have been expected to result in the detumescence of the penis. In other words, inhibition of NPY, prior to the present invention, would have been expected to maintain the penis in a flaccid state.

Surprisingly, however, it has been found by the applicants that use of NPY inhibitors, and in particular use of antagonists of NPY Y1 receptors, results in an increase in the intracavernosal pressure of the penis and, thus, facilitates and/or causes penile erection. The increase in the intracavernosal pressure resulting from the use of an NPYi, preferably an NPY Y1i, preferably occurs during sexual stimulation.

There is further provided an inhibitor which when in use is highly selective for NPY, preferably NPY Y1 receptors, associated with sexual responses, preferably in the genitalia.

The present invention further provides an NPYi, preferably an NPY Y1i, which when in use is highly selective for NPY receptors, preferably NPY Y1 receptors, associated with an increase the intracavernosal pressure in the corpus cavernosum.

The present invention further provides the use of an NPYi, preferably an NPY Y1i, in the manufacture of a medicament for selectively increasing the intracavernosal (i.c.) pressure during sexual arousal. The NPYi, preferably NPY Y1i, advantageously enhances the sexual arousally mediated increase in i.c. pressure, suitably the NPY/NPY Y1inhibitors of the present invention selectively enhance the sexually arousally mediated increase in i.c. pressure. The NPYi and/or NPY Y1i may be used to treat MED by increasing i.c. pressure, for example by influencing genital blood flow.

In particular the present invention provides an NPYi, preferably an NPY Y1i, compounds for use in the selective treatment and/or selective prevention of MED.

In addition to or as an alternative of the use of an inhibitor of a NPY Y1 receptor, an inhibitor of a NPY Y2 receptor may be used. The term NPYi used herein includes a NPY Y2 inhibitor.

The present invention is advantageous as it provides a means for restoring a normal sexual arousal response—namely increased penile blood flow leading to erection of the penis. Hence, the present invention provides a means to restore, or potentiate, the normal sexual arousal response.

Some NPYi, particularly NPY Y1i, compounds were tested as agents and were found to be useful for enhancing the endogenous erectile process, and thereby being useful in the treatment of MED. Some of the experimental data concerning an NPYi, particularly an NPY Y1i, are presented in the Experimental section (infra).

Without being limited to any particular theory it is proposed herein that by inhibiting NPY, and particularly NPY Y1, adenylate cyclase levels in and around the corpus cavernosum can be directly or indirectly enhanced. This may ultimately increase levels of cAMP in and around the corpus cavernosum. Increased levels of adenylate cyclase and/or cAMP mediates corpus cavernosal vasorelaxation and genital blood flow into the corpus cavernosum can be enhanced.

Detailed Aspects

In one aspect the present invention relates to NPYi (preferably NPY Y1i) compounds and pharmaceutical compositions comprising an NPYi, preferably an NPY Y1i, and pharmaceutical combinations consisting of an NPYi, preferably an NPY Y1i, and a PDEi, preferably a PDE5i, for use (or when in use) in the selective treatment and/or selective prevention of male sexual dysfunction, in particular MED. In the pharmaceutical compositions the NPYi or NPY Y1i (and PDEi or PDE5i, if present) is optionally admixed with a pharmaceutically acceptable carrier, diluent or excipient. Here, the composition (like any of the other compositions mentioned herein) may be packaged for subsequent use in the treatment of male sexual dysfunction, in particular MED.

In another aspect, the present invention relates to the use of an NPYi, preferably am NPY Y1i, in the manufacture of a medicament (such as a pharmaceutical composition) for use in the selective, or highly selective, treatment of male sexual dysfunction, in particular MED.

In another aspect, the present invention relates to the use of an NPYi, preferably an NPY Y1i, in the preparation of a medicament (such as a pharmaceutical composition) for use in the selective, or highly selective, treatment of male sexual dysfunction, in particular MED.

In a further aspect, the present invention relates to a medicament comprising an NPYi, preferably an NPY Y1i, which inhibitor when in use is selective for NPY, preferably NPY Y1 receptors, in genitalia.

In a further aspect, the present invention relates to a method of selectively, or highly selectively, treating or preventing MED in a human or animal which method comprises administering to an individual an effective amount of an NPYi, preferably an NPY Y1i, wherein the NPYi, preferably the NPY Y1i, is optionally admixed with a pharmaceutically acceptable carrier, diluent or excipient.

In a further aspect, the present invention relates to a method of treating a male suffering from male sexual dysfunction, in particular MED; the method comprising delivering to the male an NPYi, preferably an NPY Y1i, that is capable of selectively increasing the intracavernosal pressure during sexual arousal, without peripheral side effects.

There is further provided a pharmaceutical pack comprising one or more compartments wherein at least one compartment comprises one or more of an NPYi, preferably an NPY Y1i.

The present invention further provides a process of preparation of a pharmaceutical composition according to the present invention, said process comprising admixing one or more NPYi, preferably an NPY Y1i, with a pharmaceutically acceptable diluent, excipient or carrier.

The present invention further provides the use of an NPYi, preferably NPY Y1i, in accordance with the present invention in the manufacture or preparation of a medicament for both the selective treatment and/or selective prevention of male sexual dysfunction, particularly MED, and the treatment and/or prevention of abnormal drink and food intake disorders, in particular, obesity, anorexia, bulimia and metabolic disorders.

In a further aspect, the present invention relates to an assay method for identifying an agent (hereinafter referred to as an NPYi or an NPY Y1i) that can be used to selectively treat or prevent male sexual dysfunction, in particular MED, the assay comprising: determining whether a test agent can directly enhance the endogenous erectile process; wherein said enhancement is defined as a potentiation of intracavernosal (i.c.) pressure (and/or cavernosal blood flow) in the presence of a test agent as defined herein; such potentiation by a test agent is indicative that the test agent may be useful in the selective treatment or prevention of male sexual dysfunction, in particular MED and wherein said test agent is an NPYi, preferably an NPY Y1i. Preferably, the agent inhibits NPY, preferably NPY Y1 receptors, associated with the genitalia, particularly in association with the corpus cavernosum. Preferably, the agent has no, or substantially no, effect on arterial blood pressure.

By way of example, the present invention relates to an assay method for identifying an agent that can directly enhance the endogenous erectile process in order to treat or prevent male sexual dysfunction, in particular MED, the assay method comprising: contacting a test agent which has a moiety capable of inhibiting the metabolic breakdown of a peptide (preferably a fluorescent labelled peptide), said peptide being normally metabolised by NPY or NPY Y1; and measuring the activity and/or levels of peptide remaining after a fixed time (for example via fluorometric analysis); wherein the change in the level of the peptide measured by fluorescence is indicative of the potency (IC₅₀) of the test agent and is indicative that the test agent may be useful in the treatment or prevention of male sexual dysfunction, in particular MED; and wherein said agent is an NPYi or NPY Y1i.

In a further aspect, the present invention relates to a process comprising the steps of:

(a) performing the assay method according to the present invention;

(b) identifying one or more agents capable of inhibiting NPY, preferably NPY Y1; and

(c) preparing a quantity of those one or more identified agents; and wherein said agent is a NPYi or an NPY Y1i.

With this aspect, the agent identified in step (b) may be modified so as to maximise, for example, activity and then step (a) may be repeated. These steps may be repeated until the desired activity or pharmacokinetic profile has been achieved.

Thus, in a further aspect, the present invention relates to a process comprising the steps of: (a1) performing the assay according to the present invention; (b1) identifying one or more agents that can directly enhance the endogenous erectile process; (b2) modifying one or more of said identified agents; (a2) optionally repeating step (a1); and (c) preparing a quantity of those one or more identified agents (i.e. those that have been modified); and wherein said agent is an NPYi or an NPY Y1i.

In a further aspect, the present invention relates to a process comprising the steps of:

(i) performing the assay method according to the present invention;

(ii) identifying one or more agents capable of inhibiting NPY, preferably NPY Y1;

(iii) testing identified agents for their effect on arterial blood pressure in test animials, such as anaesthetised rabbits;

(iv) selecting agents with no, or substantially no, effect on arterial blood pressure; and

(v) preparing a quantity of those one or more selected agents; and wherein said agent is a NPYi or an NPY Y1i.

With this aspect, the agent identified in step (b) may be modified so as to maximise, for example, activity and then step (a) may be repeated. These steps may be repeated until the desired activity or pharmacokinetic profile has been achieved.

In a further aspect, the present invention relates to a method of treating or preventing male sexual dysfunction, in particular MED, by potentiating the nerve stimulated endogenous erectile process in vivo (e.g. in rabbit) by measuring the intracavernosal pressure or cavernosal blood flow with an agent; wherein the agent is capable of directly inhibiting the metabolic breakdown of a fluorescent peptide (as detailed hereinbefore) in an in vitro assay method; wherein the in vitro assay method is the assay method according to the present invention; and wherein said agent is an NPYi or an NPY Y1i.

In a further aspect, the present invention relates to the use of an agent in the preparation of a pharmaceutical composition for the selective treatment or selective prevention of male sexual dysfunction, in particular MED, wherein the agent is capable of directly inhibiting the metabolic breakdown of a fluorescent peptide when assayed in vitro by the assay method according to the present invention; and wherein said agent is an NPYi or an NPY Y1i.

In a further aspect, the present invention relates to the use of an agent in the manufacture of a pharmaceutical composition for the selective treatment or selective prevention of male sexual dysfunction, in particular MED, wherein the agent is capable of directly inhibiting the metabolic breakdown of a fluorescent peptide when assayed in vitro by the assay method according to the present invention; and wherein said agent is an NPYi or an NPY Y1i.

In a further aspect, the present invention relates to an animal model used to identify agents capable of treating or preventing male sexual dysfunction (in particular MED), said model comprising an anaesthetised male animal including means to measure changes in intracavemosal pressure and/or cavernosal blood flow of said animal following stimulation of the pelvic nerve thereof; and wherein said agent is an NPYi or an NPY Y1i.

The animal model may further comprise means to measure the arterial blood pressure of said animal.

In a further aspect, the present invention relates to an assay method for identifying an agent that can directly enhance the endogenous erectile process in order to selectively treat or selectively prevent MED, the assay method comprising: administering an agent to the animal model of the present invention; and measuring the change in the endogenous erectile process; wherein said change is defined as a potentiation of intracavernosal pressure (and/or cavernosal blood flow) in the animal model in the presence of a test agent as defined; and wherein said agent is an NPYi or an NPY Y1i.

In a further aspect, the present invention relates to an assay method for identifying an agent that can directly enhance the endogenous erectile process without effect on arterial blood pressure in order to selectively treat or selectively prevent MED, the assay method comprising: administering an agent to the animal model of the present invention; and measuring the change in the endogenous erectile process; wherein said change is defined as a potentiation of intracavernosal pressure (and/or cavernosal blood flow) in the animal model in the presence of a test agent as defined; measuring the arterial blood pressure in the animal model to ensure no or substantially no change in blood pressure; and wherein said agent is an NPYi or an NPY Y1i.

In a further aspect, the present invention relates to a diagnostic method, the method comprising isolating a sample from a male; determining whether the sample contains an entity present in such an amount as to cause male sexual dysfunction, preferably MED; wherein the entity has a direct effect on the endogenous erectile process in the corpus cavernosum of the male; and wherein said entity can be modulated to achieve a beneficial effect by use of an agent; and wherein said agent is an NPYi or an NPY Y1i.

In a further aspect, the present invention relates to a diagnostic composition or kit comprising means for detecting an entity in an isolated male sample; wherein the means can be used to determine whether the sample contains the entity and in such an amount to cause male sexual dysfunction, preferably MED, or is in an amount so as to cause male sexual dysfunction, preferably MED; wherein the entity has a direct effect on the endogenous erectile process and wherein said entity can be modulated to achieve a beneficial effect by use of an agent; and wherein said agent is an NPYi or an NPY Y1i.

Surprisingly the applicants have also found that inhibition of NPY, and particularly NPY Y1, with a NPYi, particularly a NPY Y1i, significantly potentiates PDE inhibitor, particularly PDE5 inhibitor, -mediated enhancement of the erectile process.

Since NPY and NPY Y1 receptors are present throughout the body, it is very unexpected that NPYi and/or NPY Y1i can be administered systemically and achieve a therapeutic response in the male genitalia without provoking intolerable (adverse) side effects, in particular intolerable (adverse) peripheral side effects. Thus in the in vivo (e.g. in rabbit) results hereafter the NPY Y1i alone (particularly having a selectivity as above) and NPYi/PDE5 combination when administered systemically increased genital blood flow, upon sexual arousal (mimicked by pelvic nerve stimulation) without adversely affecting cardiovascular parameters, such as causing a significant hypotensive or hypertensive effect.

Thus according to a further aspect of the invention, there is provided the use of an NPYi, preferably NPY Y1i, by systemic administration (preferably by mouth e.g. swallowable tablet or capsule, or a sublingual or buccal formulation) in the preparation of a medicament for the selective treatment or selective prevention of male sexual dysfunction, in particular MED.

Thus according to a further embodiment the present invention provides the use of a combination comprising one or more NPYi's, preferably NPY Y1i's, and one or more PDEi's, preferably PDE5i's, in the manufacture/preparation of a medicament for the selective treatment or selective prevention of male sexual dysfunction, in particular MED.

Thus according to a further embodiment the present invention provides the use of a combination consisting of one or more NPYi's, preferably NPY Y1i's, and one or more PDEi's, preferably PDE5i's, in the manufacture/preparation of a medicament for the treatment or prevention of male sexual dysfunction, in particular MED. Preferably, only NPYi's, particularly NPY Y1i's, and PDEi's, particularly PDE5i's, are used together.

Preferably said combined treatment comprises a combination of one or more NPYi's, preferably NPY Y1i's, with one or more PDEi's, preferably PDE5i's. More preferably such a combination provides for the concomitant administration of one or more NPYi's, particularly NPY Y1i's with one or more PDEi's, particularly PDE5i's, for the treatment of MED.

In a further embodiment, the present invention provides the use of a pharmaceutical composition comprising one or more NPYi's, preferably NPY Y1i's, with one or more PDEi's, preferably PDE5i's, (without the presence of further active components/ingredients, for example other inhibitors, such as neutral endopeptidase (NEP) inhibitors for example) for the treatment or prevention of MED. Preferably the pharmaceutical composition consists solely of one or more NPYi's, particularly NPY Y1i's, with one or more PDEi's, particularly PDE5i's, as the active components/ingredients for the treatment of MED.

In a further embodiment, the present invention provides the use of a pharmaceutical composition comprising one or more NPYi's, preferably NPY Y1i's, one or more PDEi's, preferably PDE5i's, and one further auxiliary active agent as disclosed hereinbelow.

According to a further embodiment, the present invention provides a pharmaceutical composition consisting of one or more NPYi's, preferably NPY Y1i's, and one or more PDEi's, preferably PDE5i's, optionally admixed with a pharmaceutically acceptable carrier, diluent or excipient.

Our results show that surprisingly this combination can be given systemically (preferably by mouth e.g. a swallowable tablet or capsule, sublingual or buccal formulation) with minimal drop in blood pressure—thus allowing systemic treatment of male sexual dysfunction using the combination.

Especially preferred for use in the pharmaceutical compositions for the selective treatment or prevention of MED according to the present invention is the combination of a potent and selective NPYi's, preferably NPY Y1i's, with a potent and selective PDEi, preferably PDE5i.

Thus, according to a further embodiment of the present invention, there is provided the use of a pharmaceutical composition consisting of one or more NPYi's, preferably NPY Y1i's, and one or more PDEi's, preferably PDE5i's, for the selective treatment of MED.

In a preferred embodiment herein said combined administration of a NPYi, preferably a NPY Y1i, and a PDEi, preferably a PDE5i, is concomitant. Concomitant administration as defined herein encompasses simultaneous (separate) administration, simultaneous combined administration, separate administration, combined administration, sequential administration and co-formulated combined administration of a PDEi (particularly PDE5i) and a NPYi (particularly NPY Y1i).

As detailed hereinbefore the present invention further proposes that, concomitant administration of a PDEi (particularly PDE5i) and NPYi (particularly NPY Y1i) can effect an increase in the efficacy as compared with that obtainable by PDE, particularly PDE5-alone associated MED therapy.

According to a further aspect of the present invention it is proposed herein that, concomitant application of an NPYi, preferably an NPY Y1i, and a PDEi, preferably a PDE5i, can provide faster onset of action than that achievable via the PDEi or PDE5i alone. In other words the present invention additionally provides the use of a fast-acting composition for the treatment of MED. A fast acting MED composition as defined herein means that following i.v. administration of the composition (consisting of a NPYi, preferably NPY Y1i, and a PDEi, preferably PDE5i) the time to maximal effect on intracavernosal pressure is reduced versus the equivalent time obtained for the same dose of the PDEi or PDE5i alone.

Thus, a further aspect of the invention provides a fast acting pharmaceutical compositions comprising an NPYi, preferably an NPY Y1i, and a PDEi, preferably a PDE5i, for use in the selective treatment of MED.

A further aspect of the invention provides a fast acting pharmaceutical compositions consisting of an NPYi, preferably an NPY Y1i, and a PDEi, preferably a PDE5i, for use in the selective treatment of MED.

It is further proposed herein that use of a NPY Y1i/PDE5i combination may enhance the efficacy of the PDE5i thereby enabling a reduction in the dose of PDE5 inhibitor required for a specific efficacy. A formulation comprising a NPY Y1i and a reduced amount of a PDE5i as defined herein means that a reduced amount of a given PDE5i is required to effect a particular response when combined with an effective amount of a NPY Y1i according to the present invention than the required amount of PDE5i alone. Such reduced dose compositions for the treatment of MED reduce the potential nitrate interactions of PDE5. Furthermore it may be desirable for particular individuals such as for example men with mild MED. This may be particularly advantageous to individuals who respond poorly to a PDE5 inhibitor alone (e.g. sildenafil).

The use of a pharmaceutical combination adapted for administering by mouth in the preparation of a medicament for the selective treatment of male sexual dysfunction, said combination comprising an inhibitor of NPY or NPY Y1 having an IC₅₀ against NPY or NPY Y1, respectively, of less than 100 nM and a selectivity for NPY or NPY Y1 receptors in the genitalia over angiotensin converting enzyme of greater than 1000, and an inhibitor of phosphodiesterase type 5 enzyme (PDE5) having an IC₅₀ against PDE5 of less than 100 nM and a selectivity for PDE5 over PDE3 of greater than 100.

Preferably, the PDE5i used herein is sildenafil, preferably sildenafil citrate.

According to a further aspect, the present invention relates to the use of a composition consisting essentially of an NPYi, preferably an NPY Y1i, as the sole active ingredient in the manufacture of a medicament (such as a pharmaceutical composition) for use in the treatment of male sexual dysfunction, in particular MED.

According to a further aspect, the present invention relates to the use of a composition consisting of an NPYi, preferably an NPY Y1i, as the sole active ingredient in the manufacture of a medicament (such as a pharmaceutical composition) for use in the treatment of male sexual dysfunction, in particular MED.

The term “active ingredient” as used herein means an ingredient which is active in the treatment of male sexual dysfunction, in particular MED.

For ease of reference, these and further aspects of the present invention are now discussed under appropriate section headings. However, the teachings under each section are not necessarily limited to each particular section.

Preferable Aspects

The agents for use in the selective treatment or selective prevention of MED according to the present invention are preferably NPY inhibitors and/or NPY Y1 inhibitors.

The agents for use in the selective treatment or selective prevention of MED according to the present invention are preferably highly selective when in use for NPY and/or NPY Y1 receptors in genitalia compared with NPY and/or NPY Y1 receptors at other peripheral locations in the body, for example in the cardiovascular system and/or the gastrointestinal system.

The agents for use in the selective treatment or prevention of MED according to the present invention in addition to being when in use highly selective for NPY and/or NPY Y1 receptors in genitalia are active against NPY and/or NPY Y1 receptors in the central nervous system and thus may be used in the treatment or prevention of both MED and food and drink intake disorders.

In one embodiment, preferably the agent for the use according to the present invention is for oral administration.

In another embodiment, the agent for the use according to the present invention may be for topical administration or intracavernosal administration.

The present invention also encompasses administration of the agent of the present invention before and/or during sexual arousal/stimulation. This is advantageous because it may provide systemic selectivity activity, such that the NPYi or NPY Y1i is active at the genitalia but not active at the cardiovascular system for example. This selectivity may be due a physiological effect rather than a pharmacological effect.

Thus, for some aspects of the present invention it is highly desirable that there is a sexual arousal/stimulation step. We have found that this step can provide systemic selectivity.

Here, “sexual arousal/stimulation” may be one or more of a visual arousal/stimulation, a physical arousal/stimulation, an auditory arousal/stimulation or a thought arousal/stimulation.

Thus, preferably the agents of the present invention are delivered before or during sexual arousal/stimulation, particularly when those agents are for oral delivery.

Hence, for this preferred aspect, the present invention provides for the use of an agent in the manufacture of a medicament for the selective treatment or selective prevention of male sexual dysfunction, in particular MED; wherein said agent is capable of inhibiting NPYi, preferably NPY Y1i, in the genitalia of an individual without peripheral side effects; and wherein said individual is sexually aroused/stimulated before or during administration of said medicament.

Preferably, the medicament is delivered orally to said individual.

In addition, for this preferred aspect, the present invention provides for a method of treating an individual; the method comprising delivering to the individual an agent that is capable of inhibiting NPYi, preferably NPY Y1i, in the genitalia without peripheral side effects; wherein the agent is in an amount to selectively treat or selectively prevent MED; wherein the agent is optionally admixed with a pharmaceutically acceptable carrier, diluent or excipient; and wherein said individual is sexually aroused/stimulated before or during administration of said agent.

Preferably said agent is delivered orally to said individual.

Surprising and Unexpected Findings

The present invention demonstrates the surprising and unexpected findings that:

(a) Inhibition of NPY receptors, particularly NPY Y1 receptors, results in an increased intracavernosal pressure and thus facilitates/causes penile erection;

(b) An agent which inhibits NPY or NPY Y1 may be useful in enhancing the erectile response and may help to overcome an erectile dysfunction such as MED without peripheral side effects;

(c) The administration of an NPYi, preferably an NPY Y1i, systemically (e.g. orally) results in selective treatment of MED without peripheral side effects, in particular without any adverse cardiovascular events, such as a drop in blood pressure.

(d) The administration of an NPYi, preferably an NPY Y1i, systemically may be useful in the selective treatment or selective prevention of both MED and food or drink intake disorders, because in addition to acting peripherally on the genitalia (although not elsewhere peripherally) the agent may also act on the central nervous system.

(e) The inhibition of NPY, and particularly NPY Y1, with a NPYi, particularly a NPY Y1i, significantly potentiates PDE inhibitor, particularly PDE5 inhibitor, -mediated enhancement of the erectile process.

Advantages

The present invention is advantageous because:

(i) Agents which inhibit NPY, particularly NPY Y1 receptors, in genitalia can provide a means for selectively preventing and/or selectively treating and/or selectively restoring a normal sexual response, such as a male erectile response, by inducing an increased intracavernosal pressure and/or blood flow into the corpus cavernosum without the risk of adverse side effects, such as adverse cardiovascular events. Hence, the present invention provides a means to restore, or mimic, the normal erectile response.

(ii) The increased intracavernosal pressure and/or blood flow into the corpus cavernosum through the inhibition of NPY, particularly NPY Y1, in combination with sexual arousal, appears to be specific to the genitalia, including the corpus cavernosum, and to have no effect on other peripheral systems, in particular, the cardiovascular system. This selective targeting reduces and/or eliminates risks and side effects (such as decreases in blood pressure) which are associated with some of the vasoactive drugs which are currently used to treat MED.

Other advantages are discussed and are made apparent hereinabove and in the following commentary.

Patient Groups

Patients with mild to moderate MED should benefit from treatment with a NPYi or NPY Y1i, and patients with severe MED may also respond. However, early investigations suggest that the responder rate of patients with mild, moderate and severe MED will be greater with a NPY or NPY Y1 inhibitor/PDE5 inhibitor combination. Mild, moderate and severe MED will be terms known to the man skilled in the art, but guidance can be found in: The Journal of Urology, vol. 151, 54-61 (January 1994).

Early investigations suggest the below mentioned MED patient groups should benefit from treatment with a NPYi/NPY Y1i and a PDE5i (or other combination set out hereinafter). These patient groups, which are described in more detail in Clinical Andrology vol. 23, no.4, p773-782 and chapter 3 of the book by I. Eardley and K. Sethia “Erectile Dysfunction-Current Investigation and Management, published by Mosby-Wolfe, are as follows: psychogenic, organic, vascular, endocrinologic, neurogenic, arteriogenic, drug-induced sexual dysfunction (lactogenic) and sexual dysfunction related to cavernosal factors, particularly venogenic causes.

NPY

As indicated above, the agent may be any suitable agent that can act as an inhibitor of NPY (sometimes referred to as an NPY antagonist).

Background teachings on NPY and its associated receptors have been prepared by Victor A. McKusick et al on http://www3.ncbi.nlm.nih.gov/Omim/searchomim.htm. The following text concerning NPY has been extracted from that source.

“Neuropeptide Y (NPY) is an abundant and widespread peptide in the mammalian nervous system. It shows sequence homology to peptide YY and over 50% homology in amino acid and nucleotide sequence to pancreatic polypeptide (PNP; 167780). NPY is a 36-amino acid peptide. Minth et al. (1984) cloned the NPY gene starting from mRNA of a pheochromocytoma. Takeuchi et al. (1985, 1986) isolated cDNA clones of the NPY and PNP genes from a pheochromocytoma and a pancreatic endocrine tumour, respectively. Using these cDNA probes to analyse genomic DNA from chromosome assignment panels of human-mouse somatic cell hybrids, they then examined the question of whether the genes are syntenic. The studies showed nonsynteny, with NPY on 7pter-7q22 and PNP on 17p11.1-17qter. By studies of a backcross with Mus spretus, Bahary et al. (1991) mapped the homologous NPY gene to mouse chromosome 6. Since mouse chromosome 6 has homology to human 7q, it is likely that the NPY gene in man is located in the region 7cen-q22. Meisler et al. (1987) excluded close linkage between the loci for cystic fibrosis (219700) and neuropeptide Y. Terenghi et al. (1987) determined the distribution of mRNA encoding NPY in neurons of the cerebral cortex in surgical biopsy specimens and postmortem brain by means of in situ hybridisation techniques. They showed consistent localisation of NPY gene transcription and expression in normal mature cortical neurons. Baker et al. (1995) showed by fluorescence in situ hybridisation that the NPY gene is located on 7p15.1 and exists in single copy. They commented that NPY is one of the most highly conserved peptides known, with, for example, only 3 amino acid differences between human and shark. Neuropeptide Y is a neuromodulator implicated in the control of energy balance and is overproduced in the hypothalamus of ob/ob mice. To determine the role of NPY in the response to leptin (164160) deficiency, Erickson et al. (1996) generated ob/ob mice deficient in NPY. In the absence of NPY, ob/ob mice were less obese because of reduced food intake and increased energy expenditure, and were less severely affected by diabetes, sterility, and somatotropic defects. These results were interpreted as indicating that NPY is a central effector of leptin deficiency. Genetic linkage analysis of rats that were selectively bred for alcohol preference identified a chromosomal region that included the NPY gene (Carr et al., 1998). Alcohol-preferring rats had lower levels of NPY in several brain regions compared with alcohol-nonpreferring rats. Thiele et al. (1998) therefore studied alcohol consumption by mice that completely lacked NPY as a result of targeted gene disruption (Erickson et al., 1996). They found that NPY-deficient mice showed increased consumption, compared with wildtype mice, of solutions containing 6%, 10%, and 20% (by volume) ethanol. NPY-deficient mice were also less sensitive to the sedative/hypnotic effects of ethanol, as shown by more rapid recovery from ethanol-induced sleep, even though plasma ethanol concentrations did not differ significantly from those of controls. In contrast, transgenic mice that overexpressed a labelled NPY gene in neurons that usually express it had a lower preference for ethanol and were more sensitive to the sedative/hypnotic effects of ethanol than controls. These data provided direct evidence that alcohol consumption and resistance are inversely related to NPY levels in the brain. As part of an on-going study of the genetic basis of obesity, Karvonen et al. (1998) identified a 1128T-C polymorphism that resulted in substitution of leucine by proline at residue 7 in the signal peptide part of pre-pro-NPY. This polymorphism was not associated with obesity or energy metabolism, but was significantly and consistently associated with high serum total and LDL cholesterol levels both in normal-weight and obese Finns and in obese Dutch subjects. Uusitupa et al. (1998) found the pro7 polymorphism in 14% of Finns but in only 6% of Dutchmen. Subjects with pro7 in NPY had, on average, 0.6 to 1.4 mmol/L higher serum total cholesterol levels than those without this gene variant. As the impact of pro7 NPY on serum cholesterol levels could not be found in normal-weight Dutchmen, it can be assumed that obese persons may be more susceptible to the effect of the gene variant. It was calculated that the probability of having the pro7 in NPY could be as high as 50 to 60% in obese subjects with a total serum cholesterol equal to or higher than 8 mmol/L. At least among Finns, the pro7 form of NPY is one of the strongest genetic factors affecting serum cholesterol levels. See also Allen and Bloom (1986); Dockray (1986); Maccarrone; and Minth et al. (1986).”

As indicated background teachings on NPY and it associated receptors have been prepared by Victor A. McKusick et al (ibid). The following text concerning NPY Y1 has been extracted from that source.

“Neuropeptide Y (NPY; 162640) is one of the most abundant neuropeptides in the mammalian nervous system and exhibits a diverse range of important physiologic activities, including effects on psychomotor activity, food intake, regulation of central endocrine secretion, and potent vasoactive effects on the cardiovascular system. Two major subtypes of NPY (Y1 and Y2) have been defined by pharmacologic criteria. The NPY Y1 receptors have been identified in a variety of tissues, including brain, spleen, small intestine, kidney, testis, placenta, and aortic smooth muscle. The Y2 receptor is found mainly in the central nervous system. Herzog et al. (1992) reported cloning of a cDNA encoding a human NPY receptor which they confirmed to be a member of the G protein-coupled receptor superfamily. When expressed in Chinese hamster ovary (CHO) or human embryonic kidney cells, the receptor exhibited characteristic ligand specificity. In the kidney cell line, the receptor was coupled to a pertussis toxin-sensitive G protein that mediated the inhibition of cyclic AMP accumulation. In the CHO cell line, on the other hand, the receptor was coupled not to inhibition of adenylate cyclase but rather to the elevation of intracellular calcium. Thus the second messenger coupling of the NPY receptor was cell type specific, depending on the specific repertoire of G proteins and effector systems present in the cell type. Larhammar et al. (1992) independently cloned and characterised the neuropeptide Y receptor. Herzog et al. (1993) determined the molecular organisation and regulation of the human NPY Y1 receptor gene. In contrast to the contiguous structure of most G protein-coupled receptor genes, they found that the NPY Y1 receptor gene has 3 exons. They also identified a common PstI polymorphism in the first intron of the gene. By high-resolution fluorescence in situ hybridisation, they localised the gene to 4q31.3-q32. Herzog et al. (1997) found that the NPY1R and NPY5R (602001) genes are colocalized on chromosome 4q31-q32. The 2 genes are transcribed in opposite directions from a common promoter region. One of the alternately spliced 5-prime exons of the Y1 receptor gene is a part of the coding sequence of the Y5 receptor. This unusual arrangement suggested to Herzog et al. (1997) that the 2 genes arose by a gene duplication event and that they may be coordinately expressed. By interspecific backcross analysis, Lutz et al. (1997) mapped the Npy1 r and Npy2r genes to conserved linkage groups on mouse chromosomes 8 and 3, respectively, which correspond to the distal region of human chromosome 4q.”

As indicated background teachings on NPY and it associated receptors has been prepared by Victor A. McKusick et al (ibid). The following text concerning NPY Y2 has been extracted from that source.

“Neuropeptide Y (NPY) signals through a family of G protein-coupled receptors present in the brain and sympathetic neurons. At least 3 types of neuropeptide Y receptor have been defined on the basis of pharmacologic criteria, tissue distribution, and structure of the encoding gene; see 162641 and 162643. Rose et al. (1995) reported the expression cloning in COS cells of a cDNA for the human type 2 receptor, NPY2R. Transfected cells showed high affinity for NPY (162640), peptide YY (PYY; 600781), and a fragment of NPY including amino acids 13 to 36. The predicted 381-amino acid protein has 7 transmembrane domains characteristic of G protein-coupled receptors and is only 31% identical to the human Y1 receptor (NPY1 R; 162641). A 4-kb mRNA was detected on Northern blots of tissue samples from several regions of the nervous system. Gerald et al. (1995) cloned the cDNA corresponding to the human Y2 receptor from a human hippocampal cDNA expression library using a radiolabeled PYY-binding assay. They expressed the Y2 gene in COS-7 cells and performed a hormone-binding assay which showed that the Y2 receptor binds (from highest to lowest affinity) PYY, NPY, and pancreatic polypeptide (PP; 167780) hormones. Ammar et al. (1996) cloned and characterised the human gene encoding the type 2 NPY receptor. The transcript spans 9 kb of genomic sequence and is encoded in 2 exons. As in the type 1 NPY receptor gene, the 5-prime untranslated region of NPY2R is interrupted by a 4.5-kb intervening sequence. Ammar et al. (1996) demonstrated by Southern analysis of rodent-human cell hybrids followed by fluorescence in situ hybridisation (FISH) that the NPY2R gene maps to 4q31, the same region containing the NPY1 R gene, suggesting that these subtypes may have arisen by gene duplication despite their structural differences. By interspecific backcross analysis, Lutz et al. (1997) mapped the Npy1r and Npy2r genes to conserved linkage groups on mouse chromosomes 8 and 3, respectively, which correspond to the distal region of human chromosome 4q.”

NPY Sequence Data

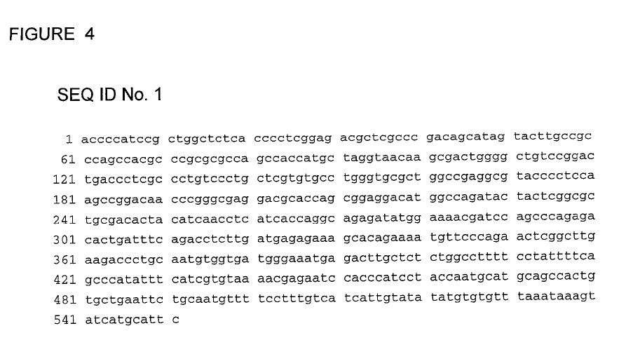

Nucleotide sequences and amino acid sequences for NPY and its receptors (i.e. NPY Y1) are available in the literature. Some sequences are presented as FIGS. 4-6.

NPY Inhibitors and/or NPY Y1 Inhibitors

Details of suitable assay systems for identifying and/or studying an NPYi (or an NPY Y1i) are presented hereinafter in the section entitled NPY assay and are based on the assays presented in WO-A-98/52890 (see page 96 thereof, lines 2 to 28).

Further examples of NPY inhibitors or NPY Y1inhibitors are disclosed and discussed in the following review articles:

Dunlop J, Rosenzweig-Lipson S: Therapeutic approaches to obesity Exp Opin Ther Pat 1999 8 12 1683-1694

Wang S, Ferguson K C, Burris T P, Dhurandhar N V: 8th annual international conference on obesity and non-insulin dependent diabetes mellitus: novel drug developments. Exp Opin Invest Drugs 1999 8 7 1117-1125

Ling A L: Neuropeptide Y receptor antagonists Exp Opin Ther Pat 1999 9 4 375-384

Adham N, Tamm J, Du P, Hou C, et al: Identification of residues involved in the binding of the antagonist SNAP 6608 to the Y5 receptor Soc Neurosci Abstr 1998 24 part 2 626.9

Shu Y Z, Cutrone J Q, Klohr S E, Huang S: BMS-192548, a tetracyclic binding inhibitor of neuropeptide Y receptors, from Aspergillus niger WB2346. II. Physico-chemical properties and structural characterization J Antibiot 1995 48 10 1060-1065

Rigollier P, Rueger H, Whitebread S, Yamaguchi Y, Chiesi M, Schilling W, Criscione L: Synthesis and SAR of CGP 71683A, a potent and selective antagonist of the neuropeptide Y Y5 receptor. Int Symp Med Chem 1998 15th Edinburgh 239

Criscione L, Rigollier P, Batzl-Hartmann C, Rueger H, Stricker-Krongrad A, et al: Food intake in free-feeding and energy-deprived lean rats is mediated by the neuropeptide Y5 receptor. J Clin Invest 1998 102 12 2136-2145

Neurogen Corp: NGD 95-1 Clin Trials Monitor 1996 5 10 Ab 19244

Buttle L A: Anti-obesity drugs: on target for huge market sales. Exp Opin Invest Drugs 1996 5 12 1583-1587

Gehiert D R, Hipskind P A: Neuropeptide Y receptor antagonists in obesity. Exp Opin Invest Drugs 1996 7 9 1827-1838

Goldstein D J, Trautmann M E: Treatments for obesity. Emerging Drugs 1997 2-1-27

Hipskind P A, Lobb K L, Nixon J A, Britton T C, Bruns R F, Catlow J, Dieckman McGinty D K, Gackenheimer S L, Gitter B D, lyengar S, Schober D A, et al.: Potent and selective 1,2,3-trisubstituted indole NPY Y-1 antagonists. J Med Chem 1997 40 3712-3714

Zimmerman D M, Cantrall B E, Smith E C R, Nixon J A, Bruns R F, Gitter B, Hipskind P A, Ornstein P L, Zarrinmayeh H, Britton T C, Schober D A, Gehlert D R: Structure-activity relationships of a series of 1-substituted-4-methylbenzimidazole neuropeptide Y-1 receptor antagonists Bioorganic Med Chem Lett 1998 8 5 473-476

Zarrinmayeh H, Nunes A, Ornstein P, Zimmerman D, Arnold M B, et al: Synthesis and evaluation of a series of novel 2-[(4-chlorophenoxy)methylbenzimidazoles as selectiveneuropeptide Y Y1 receptor antagonists J Med Chem 1998 41 15 2709-2719

Britton T C, Spinazze P G, Hipskind P A, Zimmerman D M, Zarrinmayeh H, Schober D A, Gehlert D R, Bruns R F: Structure-activity relationships of a series of benzothiophene-dervied NPY-Y1 antagonists: optimization of the C2 side chain Bioorganic Med Chem Lett 1999 9 3 475-480

Zarrinmayeh H, Zimmerman D M, Cantrell B E, Schober D A, Bruns R F, Gackenheimer S L, Ornstein P L, Hipskind P A, Britton T C, Gehlert D R: Structure-activity relationship of a series of diaminoalkyl substituted benzimidazole as neuropeptide Y Y1 receptor antagonists Bioorganic Med Chem Lett 1999 9 5 647-652

Murakami Y, Hara H, Okada T, Hashizume H, Kii M, Ishihara Y, Ishikawa M, Mihara S-I, Kato G, Hanasaki K, Hagishita S, Fujimoto M: 1,3-disubstituted benzazepines as novel, potent, selective neuropeptide Y Y1 receptor antagonists J Med Chem 1999 42 14 2621-2632

Rudolf K, Eberlein W, Engel W, Wieland H A, Willim K D, Entzeroth M, Wienen W, Beck Sickinger A G, Doods H N: The first highly potent and selective non-peptide neuropeptide YY1 receptor antagonist: BIBP3226 Eur J Pharmacol 1994 271 2-3 R11-R13

Wieland H A, Willim K D, Entzeroth M, Wienen W, Rudolf K, Eberlein W, Engel W, Doods H N: Subtype selectivity and antagonbist profile of the nonpeptide neuropeptide Y1 receptor antagonist BIBP 3226 J Pharmacol Exp Ther 1995 275 1 143-149.

Wright J, Bolton G, Creswell M, Downing D, Georgic L, Heffner T, Hodges J, MacKenzie R, Wise L: 8-amino-6-(arylsulphonyl)-5-nitroquinolones: novel nonpeptide neuropeptide Y1 receptor antagonists Bioorganic Med Chem Lett 1996 6 15 1809-1814

Capurro D, Huidobro-Toro J P: The involvement of neuropeptide Y Y1 receptors in the blood pressure baroreflex:studies with BIBP 3226 and BIB 3304. Eur J Pharmacol 1999 376 3 251-255

Dumont Y, Cadieux A, Doods H, Quirion R: New tools to investigate neuropeptide Y receptors in the central and peripheral nervous systems: BIBO-3304 (Y1), BIIE-246 (Y2) and [¹²⁵I]-GR-231118 (Y1/Y4). Soc Neurosci Abstr 1999 25 Part 1 Abs 74.11

Hegde S S, Bonhaus D W, Stanley W, Eglen R M, Moy T M, Loeb M, et al: Pharmacological evaluation of 1229U91, a high affinity and selective neuropeptide Y(NPY)—Y1 receptor antagonist Pharmacol Res 1995 31 190

Matthews J E, Chance W T, Grizzle M K, Heyer D, Daniels A J: Food intake inhibition and body weight loss in rats treated with GI 264879A, an NPY-Y1 receptor. Soc Neurosci Abstr 1997 23 Pt 2 1346

Doods H N, Willim K-D, Smith S J: BIBP 3226: a selective and highly potent NPY-Y1 antagonist Proc Br Pharmacol Soc Dec. 13-16, 1994 C47

Rudolf K, Eberlein W, Engel W, Wieland H A, Willim K D, Entzeroth M, Wienen W, Beck Sickinger A G, Doods H N: The first highly potent and selective non-peptide neuropeptide YY1 receptor antagonist: BIBP3226 Eur J Pharmacol 1994 271 2-3 R11-R13

Serradelil-Le-Gal C, Valette G, Rouby P E, Pellet A, Villanova G, Foulon L, Lespy L, Neliat G, Chambon J P, Maffrand J P, Le-Fur G: SR 120107A and SR 120819A: Two potent and selective, orally-effective antagonists for NPY Y1 receptors Soc Neurosci Abstr 1994 20 Pt 1907-Abs 376.14

Hong Y, Gregor V, Ling A L, Tompkins E V, Porter J, Chou TS, Paderes G, Peng Z, Hagaman C, Anderes K, Luthin D, May J: Synthesis and biological evaluation of novel guanylurea compounds as potent NPY Y1 receptor antagonist Acs 1999 217 Anaheim MEDI 108

Yet further examples of NPYi's and/or NPY Y1i's are disclosed in the following documents:

WO-98/07420

WO-94/00486

WO-96/22305

WO-97/20821

WO-97/20822

WO-96114307

JP-07267988

WO-96/12489

U.S. Pat. No. 5,552,422

WO-98/35957

WO-96/14307

WO-94/17035

EP-0614911

WO-98/40356

EP-0448765

EP-0747356

WO-98/35941

WO-97/46250

EP-0747357

EP-0896822

EP-1 033366

WO-00/66578

Further examples of NPY inhibitors and/or NPY Y1inhibitors are selected from the following structures:

| | | Mode of Action |
|---|---|---|
| Compound | Structure | References |
| F34 | 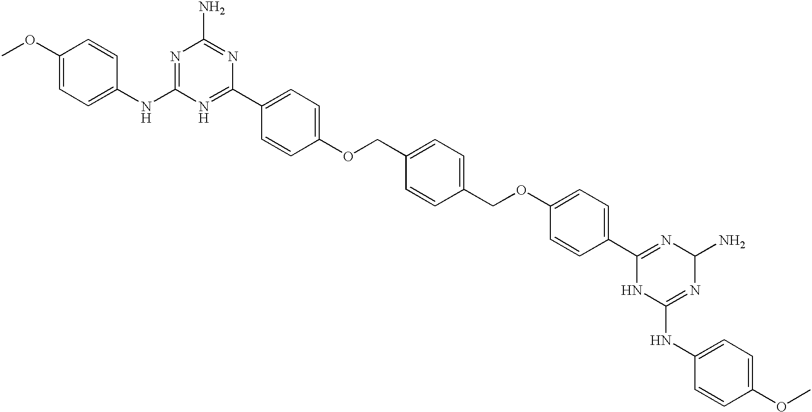 | I:NPY Y1 WO-98/07420 |
| F35 | 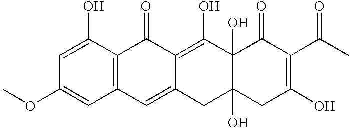 | I:NPY |
| F37 | Ile-Cys-Pro- | I:NPY Y1 |
| | Cys-Tyr-Arg-Leu-Arg-Tyr-NH2 | WO-94/00486 |
| | cyclic (2,2′), (4,4′)-disulfide dimer | WO-96/22305 |
| F39 | 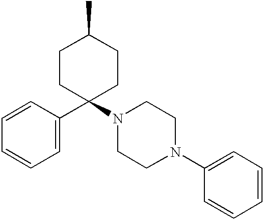 | I:NPY Y1 WO-96/14307 |
| F40 | 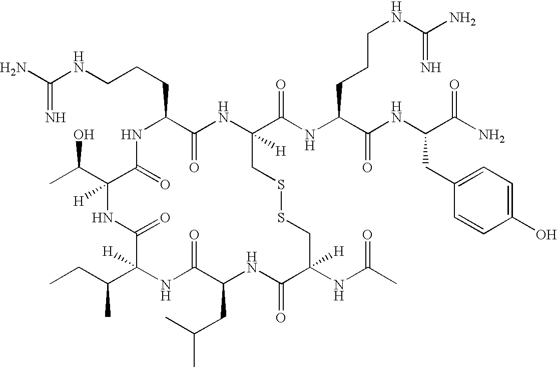 | I:NPY Y1 JP-07267988 |
| F41 | 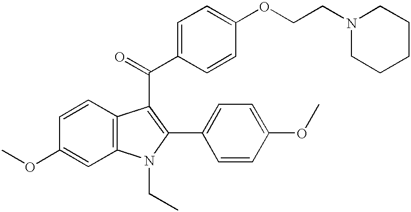 | I:NPY Y1 WO-96/12489 |
| F42 | 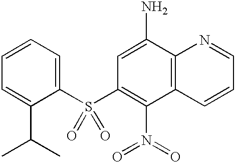 | I:NPY Y1 US-5552422 |
| F44 | 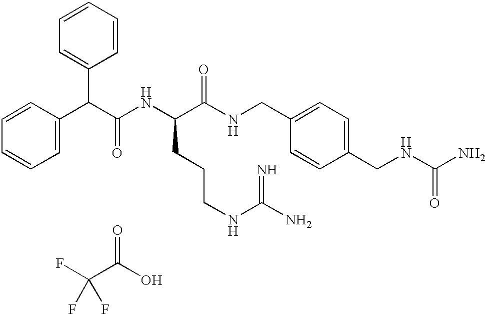 | I:NPY Y1 |
| F45 | 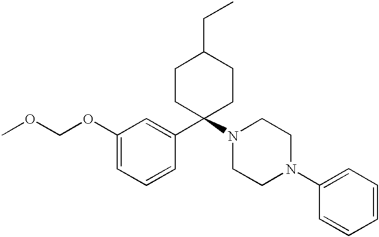 | I:NPY Y1 WO-96/14307 |
| F47a | 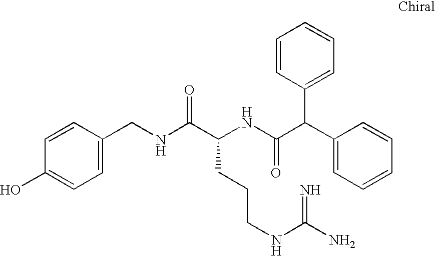 | I:NPY Y1 WO-94/17035 (BIBP 3226) |
| F48 | 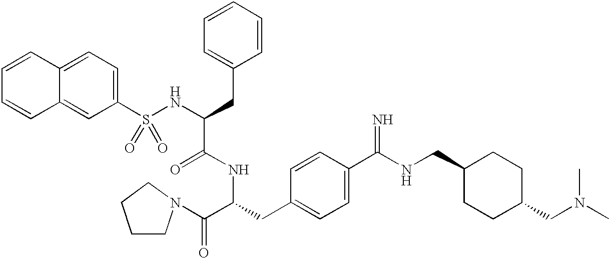 | I:NPY Y1 EP-0614911 |
| F49 | 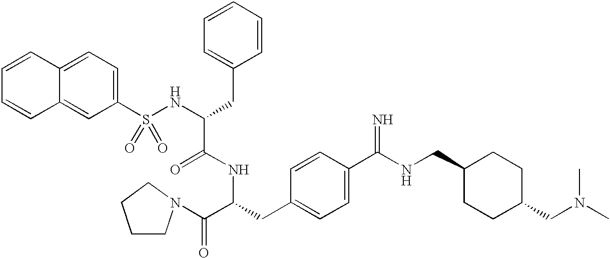 | I:NPY Y1 EP-0614911 |
| F50 | 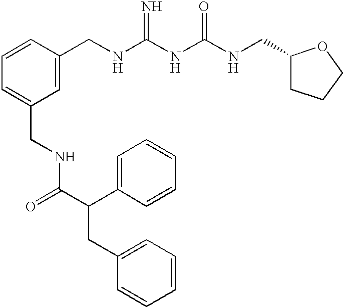 | I:NPY Y1 |
| F52 | 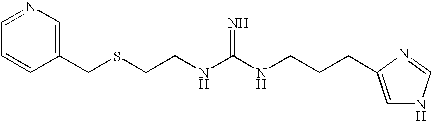 | I:NPY EP-0448765 |
| F53 | 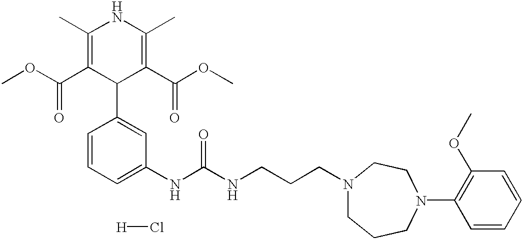 | I:NPY Y1 EP-0747356 |
| F54 | 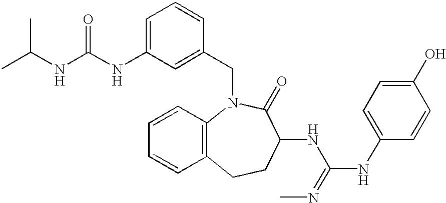 | I:NPY Y1 WO-98/35941 |
| F56 | 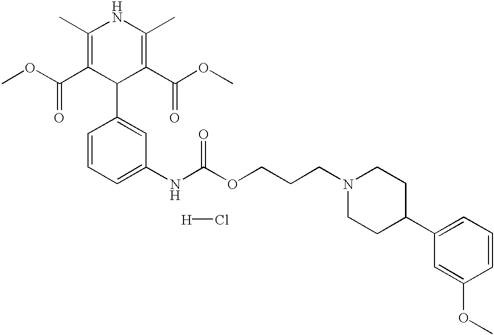 | I:NPY Y1 EP-0747357 |
| F57 | 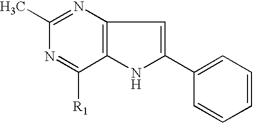 | I-NPY EP-0896822 |
| | 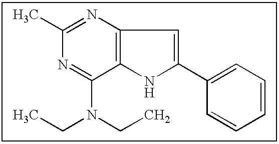 | |
| | R₁ = NR₂R₃.CO₂H, pipendino, morpholmo | |
| | R₂ = H, alkyl | |
| | R₃ = NH₂, CH₂CH═CH₂, 4 (Me)-piperazino, opt sub alkyl, Ph | |
| F58 | 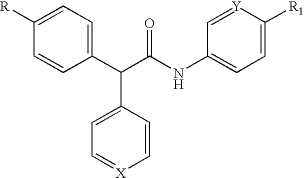 | I:NPY EP-1033366 |
| | 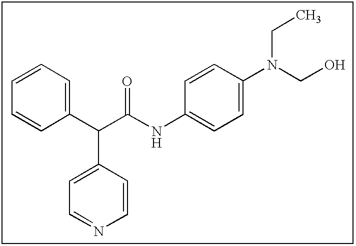 | |
| | R = H, F | |
| | R₁ = NEt, piperdino, pyrrofidino, 2,5-Me-pyrolidino, | |
| | X, Y = CH₂N | |
| F59 | 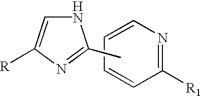 | I:NPY WO-00/66578 |
| | 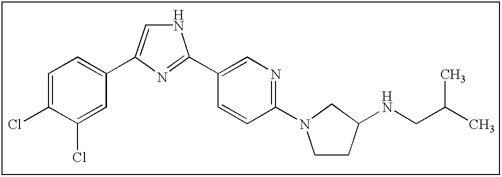 | |
| | R = opt sub Ph | |
| | R₁ = opt sub piperazino, | |
| | tetrahydropyridin-1-yl, | |
| | pyrrolidino | |

NPY Assays

As detailed in WO98/52890 (page 96, lines 2-28 thereof), the ability of compounds to bind to NPY may be assessed using a protocol essentially as described in M. W. Walker et al Journal of Neurosciences 8:2438-2446 (1988).

In this assay the cell line SK-N-MC was employed. This cell line was available from Sloane-Kettering Memorial Hospital, New York.

These cells were cultured in T-150 flasks using Dulbecco's Minimal Essential Media (DMEM) supplemented with 5% fetal calf serum. The cells were manually removed from the flasks by scraping, pelleted, and stored at −70° C.

The pellets were resuspended using a glass homogeniser in 25 mM HEPES (pH 7.4) buffer containing 2.5 mM calcium chloride, 1 mM magnesium chloride and 2 g/L bacitracin. Incubations were performed in a final volume of 200 μl containing 0.1 nM ¹²⁵I-peptide YY (2200 Ci/mmol) and 0.2-0.4 mg protein for about two hours at room temperature.

Nonspecific binding was defined as the amount of radioactivity remaining bound to the tissue after incubating in the presence of 1 μM neuropeptide Y. In some experiments various concentrations of compounds were included in the incubation mixture.

Incubations were terminated by rapid filtration through glass fibre filters which had been presoaked in 0.3% polyethyleneimine using a 96-well harvester. The filters were washed with 5 ml of 50 mM Tris (pH 7.4) at 4° C. and rapidly dried at 60° C. The filters were then treated with melt-on scintillation sheets and the radioactivity retained on the filters were counted. The results were analysed using various software packages. Protein concentrations were measured using standard coumassie protein assay reagents using bovine serum albumin as standards.

Combinations

In more detail, the present invention further comprises the combination of a compound of the invention for the treatment of male sexual dysfunction as outlined herein (more particularly male erectile dysfunction) with one or more of auxiliary active agents (see later discussion for suitable examples). The combination provides a treatment for erectile dysfunctions of organic, vascular, neurogenic, drug induced and/or psychogenic origin.

The present invention further comprises the use of a combination consisting essentially of an NPYi or an NPY Y1i according to the present invention and two auxiliary active agents (see later discussion for suitable examples) in the manufacture of a medicament for the treatment or prevention of male sexual dysfunction as outlined herein (more particularly male erectile dysfunction). The combination provides a treatment for erectile dysfunctions of organic, vascular, neurogenic, drug induced and/or psychogenic origin.

The present invention further comprises the use of a combination consisting of an NPYi or an NPY Y1i according to the present invention and two auxiliary active agents (see later discussion for suitable examples) in the manufacture of a medicament for the treatment or prevention of male sexual dysfunction as outlined herein (more particularly male erectile dysfunction). The combination provides a treatment for erectile dysfunctions of organic, vascular, neurogenic, drug induced and/or psychogenic origin.

The present invention further comprises the use of a combination consisting essentially of an NPYi or an NPY Y1i according to the present invention and one auxiliary active agent (see later discussion for suitable examples) in the manufacture or preparation of a medicament for the treatment or prevention of male sexual dysfunction as outlined herein (more particularly male erectile dysfunction). The combination provides a treatment for erectile dysfunctions of organic, vascular, neurogenic, drug induced and/or psychogenic origin.

The present invention further comprises the use of a combination consisting of an NPYi or an NPY Y1i according to the present invention and one auxiliary active agent (see later discussion for suitable examples) in the manufacture or preparation of a medicament for the treatment or prevention of male sexual dysfunction as outlined herein (more particularly male erectile dysfunction). The combination provides a treatment for erectile dysfunctions of organic, vascular, neurogenic, drug induced and/or psychogenic origin.

Thus a further combination aspect of the invention provides a pharmaceutical combination (for simultaneous, separate or sequential administration) comprising a compound of the invention and one or more auxiliary active agents (see later discussion for suitable examples).

A yet further combination aspect of the invention provides a pharmaceutical composition (for simultaneous, separate or sequential administration) consisting essentially of an NPYi or an NPY Y1i and two auxiliary active agents (see later discussion for suitable examples).

A yet further combination aspect of the invention provides a pharmaceutical composition (for simultaneous, separate or sequential administration) consisting of an NPYi or an NPY Y1i and two auxiliary active agents (see later discussion for suitable examples).

A yet further combination aspect of the invention provides a pharmaceutical composition (for simultaneous, separate or sequential administration) consisting essentially of an NPYi or an NPY Y1i and one auxiliary active agent (see later discussion for suitable examples).

A yet further combination aspect of the invention provides a pharmaceutical composition (for simultaneous, separate or sequential administration) consisting of an NPYi or an NPY Y1i and one auxiliary active agent (see later discussion for suitable examples).

Auxiliary Active Agents

Suitable auxiliary active agents for use in the combinations of the present invention include:

1) Naturally occurring or synthetic prostaglandins or esters thereof. Suitable prostaglandins for use herein include compounds such as alprostadil, prostaglandin E₁, prostaglandin E₀, 13, 14-dihydroprosta glandin E₁, prostaglandin E₂, eprostinol, natural synthetic and semi-synthetic prostaglandins and derivatives thereof including those described in WO-00033825 and/or U.S. Pat. No. 6,037,346 issued on Mar. 14, 2000 all incorporated herein by reference, PGE₀, PGE₁, PGA₁, PGB₁, PGF₁α, 19-hydroxy PGA₁, 19-hydroxy—PGB₁, PGE₂, PGB₂, 19-hydroxy-PGA₂, 19-hydroxy-PGB₂, PGE₃α, carboprost tromethamine dinoprost, tromethamine, dinoprostone, lipo prost, gemeprost, metenoprost, sulprostune, tiaprost and moxisylate;

2) α-adrenergic receptor antagonist compounds also known as α-adrenoceptors or α-receptors or α-blockers. Suitable compounds for use herein include: the α-adrenergic receptor blockerss as described in PCT application WO99/30697 published on Jun. 14, 1998, the disclosures of which relating to α-adrenergic receptors are incorporated herein by reference and include, selective α₁-adrenoceptor or α₂-adrenoceptor blockers and non-selective adrenoceptor blockers, suitable α₁-adrenoceptor blockers include: phentolamine, phentolamine mesylate, trazodone, alfuzosin, indoramin, naftopidil, tamsulosin, dapiprazole, phenoxybenzamine, idazoxan, efaraxan, yohimbine, rauwolfa alkaloids, Recordati 15/2739, SNAP 1069, SNAP 5089, RS17053, SL 89.0591, doxazosin, terazosin, abanoquil and prazosin; α₂-blocker blockers from U.S. Pat. No. 6,037,346 [Mar. 14, 2000] dibenamine, tolazoline, trimazosin and dibenamine; α-adrenergic receptors as described in U.S. Pat. Nos.: 4,188,390; 4,026,894; 3,511,836; 4,315,007; 3,527,761; 3,997,666; 2,503,059; 4,703,063; 3,381,009; 4,252,721 and 2,599,000 each of which is incorporated herein by reference; α₂-Adrenoceptor blockers include: clonidine, papaverine, papaverine hydrochloride, optionally in the presence of a cariotonic agent such as pirxamine;

3) NO-donor (NO-agonist) compounds. Suitable NO-donor compounds for use herein include organic nitrates, such as mono- di or tri-nitrates or organic nitrate esters including glyceryl trinitrate (also known as nitroglycerin), isosorbide 5-mononitrate, isosorbide dinitrate, pentaerythritol tetranitrate, erythrityl tetranitrate, sodium nitroprusside (SNP), 3-morpholinosydnonimine molsidomine, S-nitroso-N-acetyl penicilliamine (SNAP) S-nitroso-N-glutathione (SNO-GLU), N-hydroxy-L-arginine, amylnitrate, linsidomine, linsidomine chlorohydrate, (SIN-1) S-nitroso-N-cysteine, diazenium diolates,(NONOates), 1,5-pentanedinitrate, L-arginene, ginseng, zizphi fructus, molsidomine, Re—2047, nitrosylated maxisylyte derivatives such as NMI-678-11 and NMI-937 as described in published PCT application WO 0012075;

4) Potassium channel openers or modulators. Suitable potassium channel openers/modulators for use herein include nicorandil, cromokalim, levcromakalim, lemakalim, pinacidil, cliazoxide, minoxidil, charybdotoxin, glyburide, 4-amini pyridine, BaCl₂;

5) Dopaminergic agents, preferably apomorphine or a selective D2, D3 or D2/D₃agonist such as, pramipexole and ropirinol (as claimed in WO-0023056), PNU95666 (as claimed in WO-0040226);

6) Vasodilator agents. Suitable vasodilator agents for use herein include nimodepine, pinacidil, cyclandelate, isoxsuprine, chloroprumazine, halo peridol, Rec 15/2739, trazodone;

7) Thromboxane A2 agonists;

8) CNS active agents;

9) Ergot alkoloids; Suitable ergot alkaloids are described in U.S. Pat. No. 6,037,346 issued on Mar. 14, 2000 and include acetergamine, brazergoline, bromerguride, cianergoline, delorgotrile, disulergine, ergonovine maleate, ergotamine tartrate, etisulergine, lergotrile, lysergide, mesulergine, metergoline, metergotamine, nicergoline, pergolide, propisergide, proterguride and terguride;

10) Compounds which modulate the action of natruretic factors in particular atrial naturetic factor (also known as atrial naturetic peptide), B type and C type naturetic factors such as inhibitors or neutral endopeptidase;

11) Angiotensin receptor antagonists such as losartan;

12) Substrates for NO-synthase, such as L-arginine;

13) Calcium channel blockers such as amlodipine;

14) Antagonists of endothelin receptors and inhibitors or endothelin-converting enzyme;

15) Cholesterol lowering agents such as statins (e.g. atorvastatin/Lipitor-trade mark) and fibrates;

16) Antiplatelet and antithrombotic agents, e.g. tPA, uPA, warfarin, hirudin and other thrombin inhibitors, heparin, thromboplastin activating factor inhibitors;

17) Insulin sensitising agents such as rezulin and hypoglycaemic agents such as glipizide;

18) L-DOPA or carbidopa;

19) Acetylcholinesterase inhibitors such as donezipil;

20) Steroidal or non-steroidal anti-inflammatory agents;

21) Estrogen receptor modulators and/or estrogen agonists and/or estrogen antagonists, preferably raloxifene or lasofoxifene, (−)-cis-6-phenyl-5-[4-(2-pyrrolidin-1-yl-ethoxy)-phenyl]-5,6,7,8-tetrahydronaphthalene-2-ol and pharmaceutically acceptable salts thereof the preparation of which is detailed in WO 96/21656;

22) A PDE inhibitor, more particularly a PDE 2, 3, 4, 5, 7 or 8 inhibitor, preferably PDE2 or PDE5 inhibitor and most preferably a PDE5 inhibitor (see hereinafter), said inhibitors preferably having an IC50 against the respective enzyme of less than 100 nM;

23) A NEP inhibitor preferably having an IC50 for NEP of less than 300 nM, more preferably less than 100 nM;

24) Vasoactive intestinal protein (VIP), VIP mimetic, VIP analogue, more particularly mediated by one or more of the VIP receptor subtypes VPAC1, VPAC or PACAP (pituitary adenylate cyclase activating peptide), one or more of a VIP receptor agonist or a VIP analogue (eg Ro-125-1553) or a VIP fragment, one or more of a α-adrenoceptor antagonist with VIP combination (eg Invicorp, Aviptadil);

25) A melanocortin receptor agonist or modulator or melanocortin enhance, such as melanotan II, PT-14, PT-141 or compounds claimed in WO-09964002, WO-00074679, WO-09955679, WO-00105401, WO-00058361, WO-00114879, WO-00113112, WO-09954358;

26) A serotonin receptor agonist, antagonist or modulator, more particularly agonists, antagonists or modulators for 5HT1A (including VML 670), 5HT2A, 5HT2C, 5HT3 and/or 5HT6 receptors, including those described in WO-09902159, WO-00002550 and/or WO-00028993;

27) A testosterone replacement agent (including dehydroandrostendione), testosternone (Tostrelle), dihydrotestosterone or a testosterone implant;

28) Estrogen, estrogen and medroxyprogesterone or medroxyprogesterone acetate (MPA) (i.e. as a combination), or estrogen and methyl testosterone hormone replacement therapy agent (e.g. HRT especially Premarin, Cenestin, Oestrofeminal, Equin, Estrace, Estrofem, Elleste Solo, Estring, Eastraderm TTS, Eastraderm Matrix, Dermestril, Premphase, Preempro, Prempak, Premique, Estratest, Estratest HS, Tibolone);

29) A modulator of transporters for noradrenaline, dopamine and/or serotonin, such as bupropion, GW-320659;

30) A purinergic receptor agonist and/or modulator;

31) A neurokinin (NK) receptor antagonist, including those described in WO-09964008;

32) An opioid receptor agonist, antagonist or modulator, preferably agonists for the ORL-1 receptor;

33) An agonist or modulator for oxytocin/vasopressin receptors, preferably a selective oxytocin agonist or modulator;

34) Modulators of cannabinoid receptors;

35) A bombesin receptor antagonist, more particularly a bombesin BB₁, BB₂, BB₃, or BB₄ receptor antagonist, preferably a bombesin BB, inhibitor (see hereinafter), said inhibitors preferably having an IC50 against the respective enzyme of less than 100 nM;

36) A SEP inhibitor (SEPi), for instance a SEPi having an IC₅₀ at less than 100 nanomolar, more preferably, at less than 50 nanomolar.

Preferably, the SEP inhibitors according to the present invention have greater than 30-fold, more preferably greater than 50-fold selectivity for SEP over neutral endopeptidase NEP EC 3.4.24.11 and angiotensin converting enzyme (ACE). Preferably the SEPi also has a greater than 100-fold selectivity over endothelin converting enzyme (ECE);

37) An agent capable of modulating the activity of an intermediate conductance calcium-activated potassium (IK_{Ca}) channel in the sexual genitalia of an individual.

By cross reference herein to compounds contained in patents and patent applications which can be used in accordance with invention, we mean the therapeutically active compounds as defined in the claims (in particular of claim 1) and the specific examples (all of which is incorporated herein by reference).

If a combination of active agents is administered, then they may be administered simultaneously, separately or sequentially.

Auxiliary Agents—PDE5 Inhibitors

The suitability of any particular cGMP PDE5 inhibitor can be readily determined by evaluation of its potency and selectivity using literature methods followed by evaluation of its toxicity, absorption, metabolism, pharmacokinetics, etc in accordance with standard pharmaceutical practice.

IC50 values for the cGMP PDE5 inhibitors may be determined using the PDE5 assay (see hereinbelow).

Preferably the cGMP PDE5 inhibitors used in the pharmaceutical combinations according to the present invention are selective for the PDE5 enzyme. Preferably they are selective over PDE3, more preferably over PDE3 and PDE4. Preferably, the cGMP PDE5 inhibitors of the invention have a selectivity ratio greater than 100 more preferably greater than 300, over PDE3 and more preferably over PDE3 and PDE4. Selectivity ratios may readily be determined by the skilled person. IC50 values for the PDE3 and PDE4 enzyme may be determined using established literature methodology, see S A Ballard et al, Journal of Urology, 1998, vol. 159, pages 2164-2171 and as detailed herein after.

Suitable cGMP PDE5 inhibitors for the use according to the present invention include:

the pyrazolo [4,3-d]pyrimidin-7-ones disclosed in EP-A-0463756; the pyrazolo [4,3-d]pyrimidin-7-ones disclosed in EP-A-0526004; the pyrazolo [4,3-d]pyrimidin-7-ones disclosed in published international patent application WO 93/06104; the isomeric pyrazolo [3,4-d]pyrimidin-4-ones disclosed in published international patent application WO 93/07149; the quinazolin-4-ones disclosed in published international patent application WO 93/12095; the pyrido [3,2-d]pyrimidin-4-ones disclosed in published international patent application WO 94/05661; the purin-6-ones disclosed in published international patent application WO 94/00453; the pyrazolo [4,3-d]pyrimidin-7-ones disclosed in published international patent application WO 98/49166; the pyrazolo [4,3-d]pyrimidin-7-ones disclosed in published international patent application WO 99/54333; the pyrazolo [4,3-d]pyrimidin-4-ones disclosed in EP-A-0995751; the pyrazolo [4,3-d]pyrimidin-7-ones disclosed in published international patent application WO 00/24745; the pyrazolo [4,3-d]pyrimidin4-ones disclosed in EP-A-0995750; the compounds disclosed in published international application WO95/19978; the compounds disclosed in published international application WO 99/24433 and the compounds disclosed in published international application WO 93/07124. The pyrazolo [4,3-d]pyrimidin-7-ones disclosed in published international application WO 01/27112; the pyrazolo [4,3-d]pyrimidin-7-ones disclosed in published international application WO 01/27113; the compounds disclosed in EP-A-1092718 and the compounds disclosed in EP-A-1092719.

Further suitable PDE5 inhibitors for the use according to the present invention include:

5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil) also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methylpiperazine (see EP-A-0463756); 5-(2-ethoxy-5-morpholinoacetylphenyl)-1-methyl-3-n-propyl-1,6-dihydro-7H-pyrazolo[4,3-d]pyrimidin-7-one (see EP-A-0526004); 3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one (see WO98/49166); 3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one (see WO99/54333); (+)-3-ethyl-5-[5-(4-ethylpiperazin-1-ylsulphonyl)-2-(2-methoxy-1 (R)-methylethoxy)pyridin-3-yl]-2-methyl-2,6-dihydro-7H-pyrazolo[4,3-d]pyrimidin-7-one, also known as 3-ethyl-5-{5-[4-ethylpiperazin-1-ylsulphonyl]-2-([(1R)-2-methoxy-1-methylethyl]oxy)pyridin-3-yl}-2-methyl-2,6-dihydro-7H-pyrazolo[4,3-d] pyrimidin-7-one (see WO99/54333); 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, also known as 1-{6-ethoxy-5-[3-ethyl-6,7-dihydro-2-(2-methoxyethyl)-7-oxo-2H-pyrazolo[4,3-d]pyrimidin-5-yl]-3-pyridylsulphonyl}-4-ethylpiperazine (see WO 01/27113, Example 8); 5-[2-iso-Butoxy-5-(4-ethylpiperazin-1-ylsulphonyl)pyridin-3-yl]-3-ethyl-2-(1-methylpiperidin-4-yl)-2,6-dihydro-7H-pyrazolo[4,3-d]pyrimidin-7-one (see WO 01/27113, Example 15); 5-[2-Ethoxy-5-(4-ethylpiperazin-1-ylsulphonyl)pyridin-3-yl]-3-ethyl-2-phenyl-2,6-dihydro-7H-pyrazolo[4,3-d]pyrimidin-7-one (see WO 01/27113, Example 66); 5-(5-Acetyl-2-propoxy-3-pyridinyl)-3-ethyl-2-(1-isopropyl-3-azetidinyl)-2,6-dihydro-7H-pyrazolo[4,3-d]pyrimidin-7-one (see WO 01/27112, Example 124); 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one (see WO 01/27112, Example 132); (6R,12aR)-2,3,6,7,12,12a-hexahydro-2-methyl-6-(3,4-methylenedioxyphenyl) -pyrazino[2′,1′:6,1]pyrido[3,4-b]indole-1,4-dione (IC-351), i.e. the compound of examples 78 and 95 of published international application WO95/19978, as well as the compound of examples 1, 3, 7 and 8; 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil) also known as 1-[[3-(3,4-dihydro-5-methyl-4-oxo-7-propylimidazo[5,1-f]-as-triazin-2-yl)-4-ethoxyphenyl]sulphonyl]-4-ethylpiperazine, i.e. the compound of examples 20, 19, 337 and 336 of published international application WO99/24433; and the compound of example 11 of published international application WO93/07124 (EISAI); and compounds 3 and 14 from Rotella D P, J. Med. Chem., 2000, 43, 1257.

Still other suitable PDE5 inhibitors include:

4-bromo-5-(pyridylmethylamino)-6-[3-(4-chlorophenyl)-propoxy]-3 (2H)pyridazinone; 1-[4-[(1,3-benzodioxol-5-ylmethyl)amiono]-6-chloro-2-quinozolinyl]-4-piperidine-carboxylic acid, monosodium salt; (+)-cis-5,6a,7,9,9,9a-hexahydro-2-[4-(trifluoromethyl)-phenylmethyl-5-methyl-cyclopent-4,5]imidazo[2,1-b]purin-4 (3H)one; furaziocillin; cis-2-hexyl-5-methyl-3,4,5,6a,7,8,9,9a-octahydrocyclopent[4,5]-imidazo[2, 1-b]purin-4-one; 3-acetyl-1-(2-chlorobenzyl)-2-propylindole-6-carboxylate; 3-acetyl-1-(2-chlorobenzyl)-2-propylindole-6-carboxylate; 4-bromo-5-(3-pyridylmethylamino)-6-(3-(4-chlorophenyl) propoxy)-3-(2H)pyridazinone; 1-methyl-5(5-morpholinoacetyl-2-n-propoxyphenyl)-3-n-propyl-1,6-dihydro-7H-pyrazolo(4,3-d)pyrimidin-7-one; 1-[4-[(1,3-benzodioxol-5-ylmethyl)amino]-6-chloro-2-quinazolinyl]-4-piperidinecarboxylic acid, monosodium salt; Pharmaprojects No. 4516 (Glaxo Wellcome); Pharmaprojects No. 5051 (Bayer); Pharmaprojects No. 5064 (Kyowa Hakko; see WO 96/26940); Pharmaprojects No. 5069 (Schering Plough); GF-196960 (Glaxo Wellcome); E-8010 and E-4010 (Eisai); Bay-38-3045 & 38-9456 (Bayer) and Sch-51866.

In vitro PDE inhibitory activities against cyclic guanosine 3′,5′-monophosphate (cGMP) and cyclic adenosine 3′,5′-monophosphate (cAMP) phosphodiesterases were determined by measurement of their IC₅₀ values (the concentration of compound required for 50% inhibition of enzyme activity).

The required PDE enzymes were isolated from a variety of sources, including human corpus cavernosum, human and rabbit platelets, human cardiac ventricle, human skeletal muscle and human and canine retina, essentially by the method of W. J. Thompson and M. M. Appleman (Biochem., 1971, 10, 311). In particular, the cGMP-specific PDE (PDE5) and the cGMP-inhibited cAMP PDE (PDE3) were obtained from human corpus cavernosum or human platelets; the cGMP-stimulated PDE (PDE2) was obtained from human corpus cavernosum and human platelets; the calcium/calmodulin (Ca/CAM)-dependent PDE (PDE1) from human cardiac ventricle; the cAMP-specific PDE (PDE4) from human skeletal muscle and human recombinant, expressed in SF9 cells; and the photoreceptor PDE (PDE6) from human or canine retina. Phosphodiesterases 7-11 were generated from full length human recombinant clones transfected into SF9 cells.

Assays can be performed either using a modification of the “batch” method of W. J. Thompson et al. (Biochem., 1979, 18, 5228) or using a scintillation proximity assay for the direct detection of AMP/GMP using a modification of the protocol described by Amersham pic under product code TRKQ7090/7100. In summary, the effect of PDE inhibitors was investigated by assaying a fixed amount of enzyme in the presence of varying inhibitor concentrations and low substrate, (cGMP or cAMP in a 3:1 ratio unlabelled to [³H]-labelled at a conc ˜⅓ Kₘ) such that IC₅₀ ≅Kᵢ. The final assay volume was made up to 100 μl with assay buffer [20 mM Tris-HCl pH 7.4, 5 mM MgCl₂, 1 mg/ml bovine serum albumin]. Reactions were initiated with enzyme, incubated for 30-60 min at 30° C. to give <30% substrate turnover and terminated with 50 μl yttrium silicate SPA beads (containing 3 mM of the respective unlabelled cyclic nucleotide for PDEs 9 and 11). Plates were re-sealed and shaken for 20 min, after which the beads were allowed to settle for 30 min in the dark and then counted on a TopCount plate reader (Packard, Meriden, Conn.) Radioactivity units were converted to % activity of an uninhibited control (100%), plotted against inhibitor concentration and inhibitor IC₅₀ values obtained using the ‘Fit Curve’ Microsoft Excel extension (or in-house equivalent). Results from these tests show that the compounds of the present invention are inhibitors of cGMP-specific PDE5.

Functional activity can be assessed in vitro by determining the capacity of a compound of the invention to enhance sodium nitroprusside or electrical field stimulation-induced relaxation of pre-contracted rabbit corpus cavernosum tissue strips, using methods based on that described by S. A. Ballard et al. (Brit. J. Pharmacol., 1996, 118 (suppl.), abstract 153P) or S. A. Ballard et al. (J. Urology, 1998, 159, 2164-2171).

Compounds can be screened in vivo in test animals, such as anaesthetised rabbits, to determine their capacity, after i.v. administration, to enhance the pressure rises in the corpora cavernosa of the penis induced by intracavemosal injection of sodium nitroprusside, using a method based on that described by Trigo-Rocha et al. (Neurourol. and Urodyn., 1994, 13, 71).

Highly preferred for use in combination with NPYi in the pharmaceutical compositions herein are potent and selective PDE5 inhibitors.

Especially preferred herein is the combination of one or more potent and selective cGMP PDE5 inhibitors with one or more highly selective inhibitors of the NPY Y1 receptor.

Auxiliary Agents—NEP inhibitors (I:NEP=NEPi)

NEP EC3.4.24.11 (FEBS Left. 229(1), 206-210 (1988)), also known as enkephalinase or neprilysin, is a zinc-dependent neutral endopeptidase. This enzyme is involved in the breakdown of several bioactive oligopeptides, cleaving peptide bonds on the amino side of hydrophobic amino acid residues (Reviewed in Turner et al., 1997). The key neuronally released bioactive agents or neuropeptides metabolised by NEP include natriuretic peptides such as atrial natriuretic peptides (ANP) as well as brain natriuretic peptide and C-type natriuretic peptide, bombesin, bradykinin, calcitonin gene-related peptide, endothelins, enkephalins, neurotensin, substance P and vasoactive intestinal peptide. Some of these peptides have potent vasodilatory and neurohormone functions, diuretic and natriuretic activity or mediate behaviour effects. Background teachings on NEP have been presented by Victor A. McKusick et al on http://www3.ncbi.nlm.nih.gov/Omim/searchomim. htm.

The suitability of any particular I:NEP can be readily determined by evaluation of its potency and selectivity using literature methods followed by evaluation of its toxicity, absorption, metabolism, pharmacokinetics, etc in accordance with standard pharmaceutical practice.

Preferably the I:NEP have a selectivity over ACE of greater than 300.

IC50 values and selectivity ratios for ACE may be determined by methods described in EP1097719A1.

Examples of NEP inhibitors are disclosed and discussed in the following review articles: Pathol. Biol., 46(3), 1998, 191; Current Pharm. Design, 2(5), 1996, 443; Biochem. Soc. Trans., 21(3), 1993, 678; Handbook Exp. Pharmacol., 104/1, 1993, 547; TiPS, 11, 1990, 245; Pharmacol. Rev., 45(1), 1993, 87; Curr. Opin. Inves. Drugs, 2(11), 1993, 1175; Antihypertens. Drugs, (1997), 113; Chemtracts, (1997), 10(11), 804; Zinc Metalloproteases Health Dis. (1996), 105; Cardiovasc. Drug Rev., (1996), 14(2), 166; Gen. Pharmacol., (1996), 27(4), 581; Cardiovasc. Drug Rev., (1994), 12(4), 271; Clin. Exp. Pharmacol. Physiol., (1995), 22(1), 63; Cardiovasc. Drug Rev., (1991), 9(3), 285; Exp. Opin. Ther. Patents (1996), 6(11), 1147.

Further examples of NEP inhibitors are disclosed in the following documents: EP-509442A; U.S. Pat. Nos. 192,435; 4,929,641; EP-599444B; U.S. Pat. No. 884,664; EP-544620A; U.S. Pat. No. 798,684; J. Med. Chem. 1993, 3821; Circulation 1993, 88(4), 1; EP-136883; JP-85136554; U.S. Pat. No. 4,722,810; Curr. Pharm. Design, 1996, 2, 443; EP-640594; J. Med. Chem. 1993, 36(1), 87; EP-738711-A; JP-270957; CAS #115406-23-0; DE-19510566; DE-19638020; EP-830863; JP-98101565; EP-733642; WO9614293; JP-08245609; JP-96245609; WO9415908; JP05092948; WO-9309101; WO-9109840; EP-519738; EP-690070; J. Med. Chem. (1993), 36, 2420; JP-95157459; Bioorg. Med. Chem. Letts., 1996, 6(1), 65; EP-A-0274234; JP-88165353; Biochem.Biophys.Res. Comm., 1989, 164, 58; EP-629627-A; U.S. Pat. No. 77,978; Perspect. Med. Chem. (1993), 45; EP-358398-B

Further examples of NEP inhibitors are disclosed in EP1097719-A1, in particular compounds FXII to FXIII therein.

Preferred NEP inhibitors are compounds FV to FXI and F57 to F65 of EP1097719-A1.

Auxiliary Agents—Bombesin Receptor Antagonists

It has been found that bombesin receptor antagonists are useful in the treatment of male sexual dysfunction, especially drug-induced male sexual dysfunction and psychogenic male sexual dysfunction associated with generalised unresponsiveness and ageing-related decline in sexual arousability.

Compounds that are bombesin receptor antagonists have been tested using animal models that are believed to be reliable and predictive, in particular with the capacity to make predictions for females. In rodents proceptive behaviour is under hormonal control, progesterone being essential for induction of proceptive behaviour in combination with oestrogen (Johnson M and Everitt B., Essential Reproduction (3^{rd} edn), Blackwell, Oxford, 1988). The evidence for the hormonal control of proceptive behaviour in primates is conflicting, but on the whole oestrogens and/or androgens appear to enhance proceptive behaviour (Baum M. J., J. Biosci., 1983; 33:578-582). The behavioural manifestations of proceptive behaviour in the rat include “hopping and darting” movement, with rapid vibration of the ears. Tests to assess the eagerness to seek sexual contact (sexual motivation) have been reported as the most appropriate way to measure proceptivity (Meyerson B. J, Lindstrom L. H., Acta Physiol. Scand., 1973; 389 (Suppl.): 1-80). Receptivity, in the rat, is demonstrated when the female assumes a lordotic position. This occurs when, on mounting, the male exerts pressure with his forepaws on the flanks of the receptive female. The main sites of neuronal control for this behaviour are the ventromedial nucleus (VMN) and the midbrain central grey area (MCG) (for review, see Wilson C. A., In: Sexual Pharmacology, Riley A. J. et al, (Eds), Clarendon Press, Oxford, 1993: 1-58).

Bombesin is a 14-amino acid peptide originally isolated from the skin of the European frog Bombina bombina (Anastasi A. et al., Experientia, 1971; 27: 166). It belongs to a class of peptides which share structural homology in their C-terminal decapeptide region (Dutta A. S., Small Peptides; Chemistry, Biology, and Clinical Studies, Chapter 2, pp 66-82). At present, two mammalian bombesin-like peptides have been identified, the decapeptide neuromedin B (NMB) and a 23-residue amino acid, gastrin-releasing peptide (GRP).

Bombesin evokes a number of central effects through actions at a heterogeneous population of receptors. The BB, receptor binds neuromedin B (NMB) with higher affinity than gastrin-related peptide (GRP) and neuromedin C (NMC) and BB₂ receptors bind GRP and NMC with greater affinity than NMB. More recently evidence has emerged of two more receptor subtypes denoted BB₃ and BB₄ but due to limited pharmacology, little is known of their function at present. BB₁ and BB₂ receptors have a heterogeneous distribution within the central nervous system indicating that the endogenous ligands for these receptors may differentially modulate neurotransmission. Among other areas, BB₁ receptors are present in the ventromedial hypothalamus (Ladenheim E. E et al, Brain Res., 1990; 537: 233-240).

Bombesin-like immunoreactivity and mRNA have been detected in mammalian brain (Braun M., et al., Life. Sci., 1978; 23: 2721) (Battey J., et al., TINS, 1991;14:524). NMB and GRP are believed to mediate a variety of biological actions (for a review, see WO 98/07718).

The following patent applications disclose compounds capable of antagonising the effects of NMB and/or GRP at bombesin receptors: CA 2030212, EP 0309297, EP 0315367, EP 0339193, EP 0345990, EP 0402852, EP 0428700, EP 0438519, EP 0468497, EP 0559756, EP 0737691, EP 0835662, JP 07258081, UK 2231051, U.S. Pat. Nos. 4,943,561, US 5,019,647, US 5,028,692, US 5,047,502, US 5,068,222, US 5,084,555, US 5,162,497, US 5,244,883, US 5,439,884, US 5,620,955, US 5,620,959, US 5,650,395, US 5,723,578, US 5,750,646, US 5,767,236, US 5,877,277, US 5,985,834, WO 88/07551, WO 89/02897, WO 89/09232, WO 90/01037, WO 90/03980, WO 91/02746, WO 91/04040, WO 91/06563, WO 92/02545, WO 92/07830, WO 92/09626, WO 92/20363, WO 92/20707, WO 93/16105, WO 94/02018, WO 94/02163, WO 94/21674, WO 95/00542, WO 96/17617, WO 96/28214, WO 97/09347, WO 98/07718, WO 00/09115, WO 00/09116. We believe that compounds disclosed in these applications can be used in the prevention or treatment of sexual dysfunction, which is an indication that is not disclosed or suggested by the aforesaid applications, or indeed in any previous scientific publication concerning bombesin receptors.

One preferred genus of bombesin receptor antagonists disclosed in WO 98/07718 comprises compounds of the formula (I)

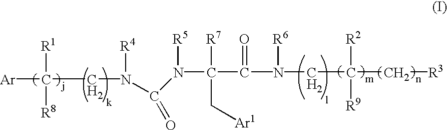

and pharmaceutically acceptable salts thereof, wherein:

j is 0 or 1;

k is 0 or 1;

l is 0, 1, 2, or 3;

m is 0 or 1;

n is 0, 1 or 2;

Ar is phenyl, pyridyl or pyrimidyl, each unsubstituted or substituted by from 1 to 3 substituents selected from alkyl, halogen, alkoxy, acetyl, nitro, amino, —CH₂NR¹⁰R¹¹, cyano, —CF₃, —NHCONH₂, and —CO₂R¹²;

R¹ is hydrogen or straight, branched, or cyclic alkyl of from 1 to 7 carbon atoms;

R⁸ is hydrogen or forms a ring with R¹ of from 3 to 7 carbon atoms;

R² is hydrogen or straight, branched, or cyclic alkyl of from 1 to 8 carbon atoms which can also contain 1 to 2 oxygen or nitrogen atoms;

R⁹ is hydrogen or forms with R² a ring of from 3 to 7 carbon atoms which can contain an oxygen or nitrogen atom; or R² and R⁹ can together be a carbonyl;

Ar¹ can be independently selected from Ar and can also include pyridyl-N-oxide, indolyl, imidazolyl, and pyridyl;

R⁴, R⁵, R⁶, and R⁷ are each independently selected from hydrogen and lower alkyl; R⁴ can also form with R⁵ a covalent link of 2 to 3 atoms which may include an oxygen or a nitrogen atom;

R³ can be independently selected from Ar or is hydrogen, hydroxy, —NMe₂, N-methyl-pyrrolyl, imidazolyl, N-methyl-imidazolyl, tetrazolyl, N-methyl-tetrazolyl, thiazolyl, —CONR¹³R¹⁴, alkoxy,

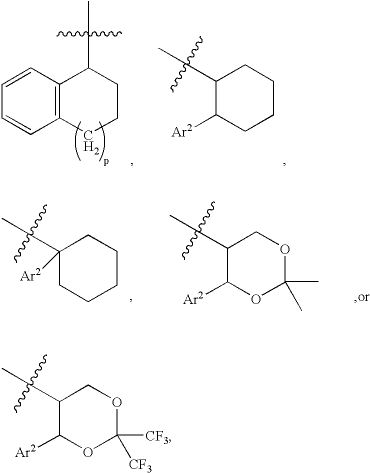

wherein p is 0, 1 or 2 and Ar² is phenyl or pyridyl;

R¹⁰, R¹¹, R¹², R¹³ and R¹⁴ are each independently selected from hydrogen or straight, branched, or cyclic alkyl of from 1 to 7 carbon atoms.

A particularly preferred compound within the above genus is (S) 3-(1H-Indol-3-yl)-N-[1-(5-methoxy-pyridin-2-yl)-cyclohexylmethyl]-2-methyl-2-[3-(4-nitro-phenyl)-ureido]-propionamide and its pharmaceutically acceptable salts.

BB₁ AND BB₂ Binding Assays

In the following experiments, measurement of BB₁ and BB₂ binding was as follows. CHO-K1 cells stably expressing cloned human NMB (for (BB₁ assay) and GRP receptors (for BB₂ assay) were routinely grown in Ham's F12 culture medium supplemented with 10% foetal calf serum and 2 mM glutamine. For binding experiments, cells were harvested by trypsinization, and stored frozen at −70° C. in Ham's F12 culture medium containing 5% DMSO until required. On the day of use, cells were thawed rapidly, diluted with an excess of culture medium, and centrifuged for 5 minutes at 2000 g. Cells were resuspended in 50 mM Tris-HCl assay buffer (pH 7.4 at 21° C., containing 0.02% BSA, 40 μg/mL bacitracin, 2 μg/mL chymostatin, 4 μg/mL leupeptin, and 2 μM phosphoramidon), counted, and polytronned (setting 5, 10 sec) before centrifuging for 10 minutes at 28,000 g. The final pellet was resuspended in assay buffer to a final cell concentration of 1.5×10⁵/mL. For binding assays, 200 μL aliquots of membranes were incubated with [¹²⁵I] [Tyr⁴]bombesin (<0.1 nM) in the presence and absence of test compounds (final assay volume 250 μL) for 60 minutes and 90 minutes for NMB and GRP receptors, respectively. Nonspecific binding was defined by 1 μM bombesin. Assays were terminated by rapid filtration under vacuum onto Whatman GF/C filters presoaked in 0.2% PEI for >2 hours, and washed 50 mM Tris-HCl (pH 6.9 at 21° C.; 6 □1 mL). Radioactivity bound was determined using a gamma counter.

All competition data was analysed using nonlinear regression utilizing iterative curve-plotting procedures in Prism® (GraphPad Software Inc., San Diego, USA). IC₅₀ values were corrected to Kᵢ values using the Cheng-Prusoff equation (Cheng Y., Prusoff W. H., Biochem. Pharmacol. 22: 3099-3108,1973).

Auxiliary Agents—SEP Inhibitors (SEPi)

A SEPi is a compound which inhibits or selectively inhibits a polypeptide having SEP activity.

SEP is a soluble secreted endopeptidase. Endopeptidases, including serine proteases, cysteine proteases and metalloendopeptidases, cleave at a sequence within an peptide.

An important group of endopeptidases known as zinc metalloproteases are characterised by having a requirement for the binding of a zinc ion in their catalytic site. Zinc metalloproteases can be subdivided into classes (for review see FEBS Letters 354 (1994) pp. 1-6), with one such class being the neprilysin (NEP)-like zinc metalloproteases (FASEB Journal, Vol 11, 1997 pp. 355-384). The NEP class includes at least 7 enzymes that are structurally related to each other (see later). They are typically membrane-bound, with a large carboxy-terminal extracellular domain, a short membrane-spanning region, and a short intracellular domain at the amino terminus. Known members of this family are neprilysin (also called NEP, CD10, CALLA, enkephalinase or EC 3.4.24.11), endothelin-converting enzymes (ECE-1 and ECE-2), PEX, KELL, X-converting enzyme/damage induced neural endopeptidase (XCE/DINE), and an enzyme identified in rodents called soluble secreted endopeptidase/neprilysin II (SEP/NEPII; Ghaddar, G et al, Biochem Journal, Vol 347, 2000, pp. 419-429; Ikeda, K et al, Journal Biological Chemistry, Vol 274, 1999, pp. 32469-32477; Tanja, 0 et al, Biochem Biophys Research Communication, Vol 271, 2000, pp. 565-570; International Patent Application WO 99/53077). The functions of the members of this class are thought to be related to peptidergic signalling. This is a process that occurs in most organisms, including humans, in which peptide molecules are used as “messengers” to elicit physiological responses. This typically involves the production and release of the peptide messenger by a specific cell, sometimes as an inactive precursor that is cleaved by a protease to become active. The active form of the peptide then binds a specific receptor on the surface of another cell where it elicits a response. The peptide is then inactivated by degradation by another protease.

NEPII is likely to be a rat equivalent of SEP, which is a mouse enzyme, as they share 91% amino acid identity. They are the members of this class closest to NEP in their amino acid sequence, both being 54% identical to human NEP. The mRNA of both is highly abundant in the testis and can also be detected at low levels in a broad range of other tissues. In the case of rat NEPII, the mRNA has also been found at comparatively high levels in the brain and pituitary. When produced recombinantly in mammalian cells, both mouse SEP and rat NEPII can be found in the growth media. This suggests they could be secreted proteases that may be able to circulate and hence cleave peptides at other sites in the body. Mouse SEP and rat NEPII, like some other members of this class such as ECE-1, exhibit splice variation. In the case of mouse SEP and rat NEPII, this splice variation results in isoforms with alterations in sequences involved in membrane localisation and secretion. The physiological significance of this is unclear but it is likely there could be membrane-bound, circulating, and intracellular forms of these enzymes. Mouse SEP has been shown to be able to cleave a range of important biological peptides including enkephalin, endothelin, big-endothelin, Bradykinin and substance P. Like NEP, therefore, it has a fairly broad substrate specificity and may have several physiological functions in different tissues.

Enzymes in this NEP class, like other metalloprotease enzymes, have been shown to be amenable to inhibition by small drug-like molecules (for example, thiorphan and phosphoramidon). This, together with the emerging nature of the physiological function of some members of the NEP-like enzymes in modulating peptidergic signalling, makes them attractive targets for pharmaceutical intervention.

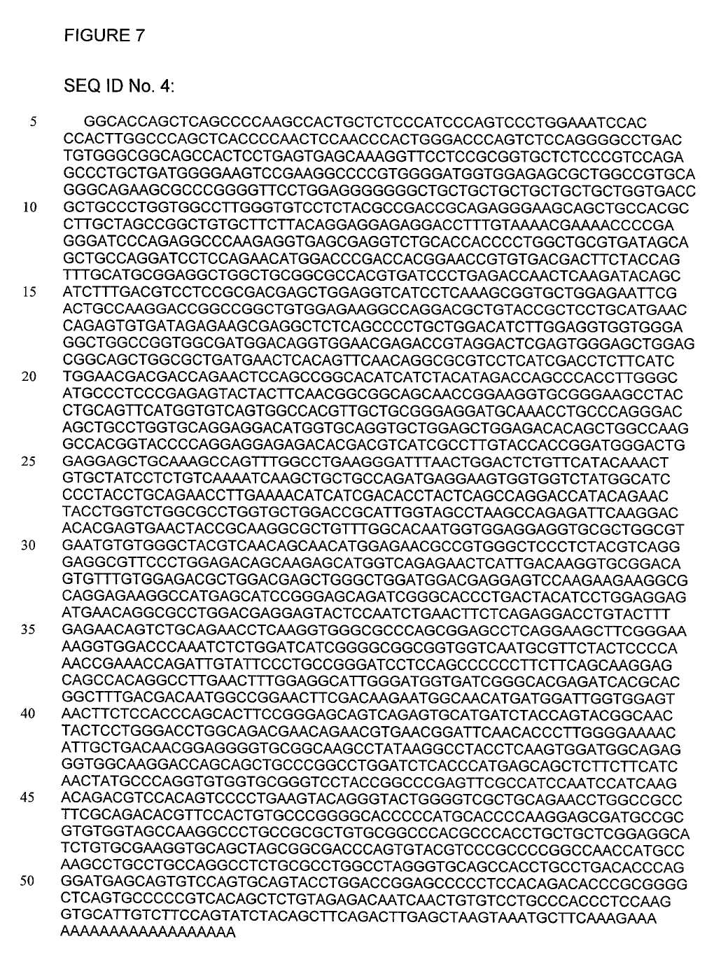

Sequences for SEP are presented in WO99/53077, EP 1069188 and WO0/47750 and also in FIGS. 7-9 (SEQ ID No.s 4-6).

SEP sequences mentioned herein for, for example, assays, include references to any one or more of the sequences presented in WO99/53077, EP 1069188 or WO00/47750 or presented as SEQ ID No. 4, SEQ ID No. 5 or SEQ ID No. 6 or variants, fragments, homologues, analogues or derivatives thereof. SEQ ID No. 4 and SEQ ID No. 5 each disclose a nucleotide sequence (cDNA) coding for human SEP. SEQ ID No. 5 includes 5′ and 3′ partial vector sequences. SEQ ID No. 6 shows a human SEP protein.

The suitability of any particular SEPi can be determined by evaluation of its potency and selectivity using, for example, the following assays followed by evaulation of its toxicity, absorption, metabolism, pharmacokinetics, etc. in accordance with standard pharmaceutical practice.

One SEP assay that may be used to detect candidate inhibitors of SEP is a fluorescence resonance energy transfer (FRET) assay. Most preferably, said labelled substrate peptide is Rhodamine green-Gly-Gly-dPhe-Leu-Arg-Arg-Val-Cys(QSY™-7)-βAla-NH₂.

SEP FRET Assay

The SEP FRET assay is based on an assay developed by Carvalho et al. for use with NEP (Carvalho et al., Annal. Biochem. 237, pp. 167-173 (1996)). The SEP FRET assay utilises a similar intramolecularly quenched fluorogenic peptide substrate, but with a novel combination of fluorogenic donor/acceptor dyes, specifically Rhodamine green (Molecular Probes, Inc., Eugene, Oreg., USA) and QSY™-7 (abbreviated hereafter as “QSY-7” or “QSY7”; Molecular Probes, Inc.).

The endopeptidase activity of SEP is measured by monitoring its ability to proteolyse the synthetic peptide substrate Rhodamine green-Gly-Gly-dPhe-Leu-Arg-Arg-Val-Cys(QSY7)-βAla-NH₂.

The two fluorophores (fluorogenic dyes) chosen for this assay have overlapping emission and absorption spectra and hence are suitable for energy transfer. The Rhodamine green acts as a donor and when excited at 485 nm gives out an emission (fluorescence) at 535 nm which in turn excites the QSY7 (FRET is occurring). The QSY7 is fluorescently silent and so gives off no emission above 535 nm hence no signal is observed (the Rhodamine green emission is quenched).

Upon cleavage (selective hydrolysis) by SEP at the Arg-Val peptide bond of the peptide substrate, the Rhodamine green and QSY7 moieties move apart and so upon excitation at 485 nm, energy transfer can no longer take place. As a result, an increase in fluoresence is observed at 535 nm for the Rhodamine green.

Preparation of the Synthetic Peptide Substrate Rhodamine green-Gly-Gly-dPhe-Leu-Arg-Arg-Val-Cys(QSY7)-βAla-NH₂

Peptide assembly was completed on 0.25 mmol FMOC-PAL-PEG-PS resin by solid phase peptide synthesis protocols using modifications to manufacturer supplied (Applied Biosystems, Foster City, Calif., USA) 9-fluoreneylmethoxycarbonyl (FMOC)-based synthesis cycles. Our modified cycles deprotect the amino terminus with 2×5 minute treatments with 20% piperidine/N-methylpyrrolidinone (NMP); the efficiency of which is monitored by UV absorbance at 301 nm by passage of a small aliquot of deprotection solution through a UV absorbance detector. In a separate cartridge, the incoming amino acid is activated with 0.9 equivalents each of 2-(1H-Benzotriazole-1-yl)-1,1,3,3 tetramethyluronium hexafluorophosphate (HBTU)/1-Hydroxybenzotriazole (HOBt) dissolved in N,N-dimethylformamide (DMF). 2 equivalents of diisopropylethyl amine (DIEA) are added. Concurrently, the resin is then washed with NMP to remove deprotection by-products. The wash solution is drained from the resin and the activated amino acid ester is transferred to the resin and stirred to allow coupling to the amino terminus for 20 minutes. The residual coupling solution is drained and the resin washed again with NMP. To ensure peptide homogeneity, a solution of 0.4M Acetic Anhydride/0.04M HOBt in NMP and 12 mmole DIEA are added to the resin to acetylate any potential unreacted sites. Finally, the resin is washed with NMP, drained, then washed with a mixture of 1:1 dichloromethane/2,2,2-trifluoroethanol and drained. This typifies one cycle of peptide synthesis. The completed synthesis resin was cleaved and deprotected using Reagent K (King, D. S. et. al., (1990), Int J. Pep. Prot Res., 36, pp. 255-66) affording 251 mg (100%) crude peptide CPI Electrospray mass spectrometry (ESMS) (m/z calculation (calc.)=977.21 (MH+average), obs. =977.47).

Attachment of QSY-7 to Cysteine:

50 mg (51 μmol) of crude CP1 was dissolved in solution of 10% DIEA/DMF containing 45 mg (52.4mmol) QSY-7 maleimide After 10 minutes, the reaction was judged to be incomplete via HPLC-MS analysis and an additional 30 mg (30.7 μmol) crude peptide was added. After 30 additional minutes, the reaction was judged via HPLC-MS to be complete and all starting reagents consumed. The product was isolated by C18 preparative HPLC chromatography and fractions exhibiting desired product molecular weight by Matrix Assisted Laser Desorption Ionization mass spectrometry (MALDI-MS) were pooled and lyophilized to 73.7 mg (50%) of a purple powder, CP2 ESMS (m/z calc.=1797.86 (MH+monoisotopic), obs.=1797.86).

Attachment of bis(trifluoroacetyl) Rhodamine Green to the Amino Terminus:

73.7 mg (41 μmol) of CP2 was dissolved in a 2% DIEA/DMF solution containing 35 mg (52.8 μmol) Rhodamine Green carboxylic acid, trifluoroacetamide, succinimidyl ester (5(6)-CR 110 TFA, SE)*mixed isomers*. After 2 hours, the reaction was judged to be complete via HPLC-MS analysis. The product was isolated via C4 preparative HPLC chromatography and fractions exhibiting desired product molecular weights (MALDI-MS) were pooled and lyophilized to 71.4 mg (74%) of a purple powder CP3 ESMS (m/z calc.=2345.92 (MH+monoisotopic), obs.=2345.47).

Removal of Trifluoroacetyl Protecting Groups from Rhodamine Green:

71.4 mg (30.4 μmol) of CP3 was dissolved in 10 ml 4:1 CH₃CN/H₂O. To this was added 200 mg (1886 μmol) Na₂CO₃. After 16 hr. vortexing, the supernatant was decanted from the insoluble material. The reaction vessel was rinsed with 1 ml DMSO; this was combined with the supernatant and the product isolated via C4 preparative HPLC chromatography. Fractions exhibiting product molecular weights (MALDI-MS) were combined and lyophilized to 64 mg (98%) of a purple powder, CP4 ESMS (m/z calc.=2155.54 (MH+average), obs.=2155.27). CP4 is the desired synthetic peptide substrate Rhodamine green-Gly-Gly-dPhe-Leu-Arg-Arg-Val-Cys(QSY7)-βAla-NH₂.

Materials:

All reagents were purchased of the highest commercial purity available and were used without further refinement. All reagents for peptide synthesis were purchased from Applied Biosystems, Foster City, Calif., USA with the following exceptions: QSY™-7 maleimide (Catalog number Q-10257) and Rhodamine Green carboxylic acid, trifluoroacetamide, succinimidyl ester (5(6)-CR 110 TFA, SE)*mixed isomers* (Catalog number R-6112) were purchased from Molecular Probes, Inc., OR, USA; FMOC-PAL-PEG-PS was purchased from Perseptive Biosystems, MA, USA (Catalog number GEN913384); FMOC-B-Alanine and FMOC-d-phenylalanine were purchased from Novabiochem, CA, USA; FMOC-Arg(Pbf)-OH was purchased from AnaSpec, Inc., CA, USA; 2,2,2-Trifluoroethanol was purchased from Aldrich, WI, USA. Sodium Carbonate was purchased from Fisher, PA, USA.

Preparative HPLC chromatography was performed on Vydac (CA, USA) C18 (Catalog number 218TP1022) or C4 (Catalog number 214TP1022) columns at 10 ml/min flow rate eluting with a linear gradient of 0% to 80% (A=5% CH₃CN/0.1% TFA/94.9% H₂O, B=100% CH₃CN) over 30 minutes collecting 30 second time fractions. Analytical HPLC-MS was performed using a Micromass (Manchester, UK) LCT mass spectrometer (masses based on externally calibrated standards) coupled with a Waters (MA, USA) 2690 HPLC inlet and a Waters 996 photodiode array detector performing chromatography on a Vydac C4 (Catalog number 214TP5415) column with a linear gradient of 0% to 80% (A=5% CH₃CN/0.1% TFA/94.9% H₂O, B=100% CH₃CN) over 30 minutes at 1 ml/min flow rate. Deconvoluted molecular weights were calculated from multiply charged observed ions using Micromass transform software. MALDI-MS were obtained on a Perseptive Biosystems Voyager-DE linear mass spectrometer using alpha cyano 4-hydroxy cinnamic acid matrix (Hewlett Packard, CA, USA) and reported masses based on external calibration.

Process (Including Chemical Structures):

CP4 (=synthetic peptide substrate Rhodamine green-Gly-Gly-dPhe-Leu-Arg-Arg-Val-Cys(QSY™-7)-βAla-NH₂) is synthesised by incorporating the key intermediate CP3 in a solid phase peptide synthesis scheme.

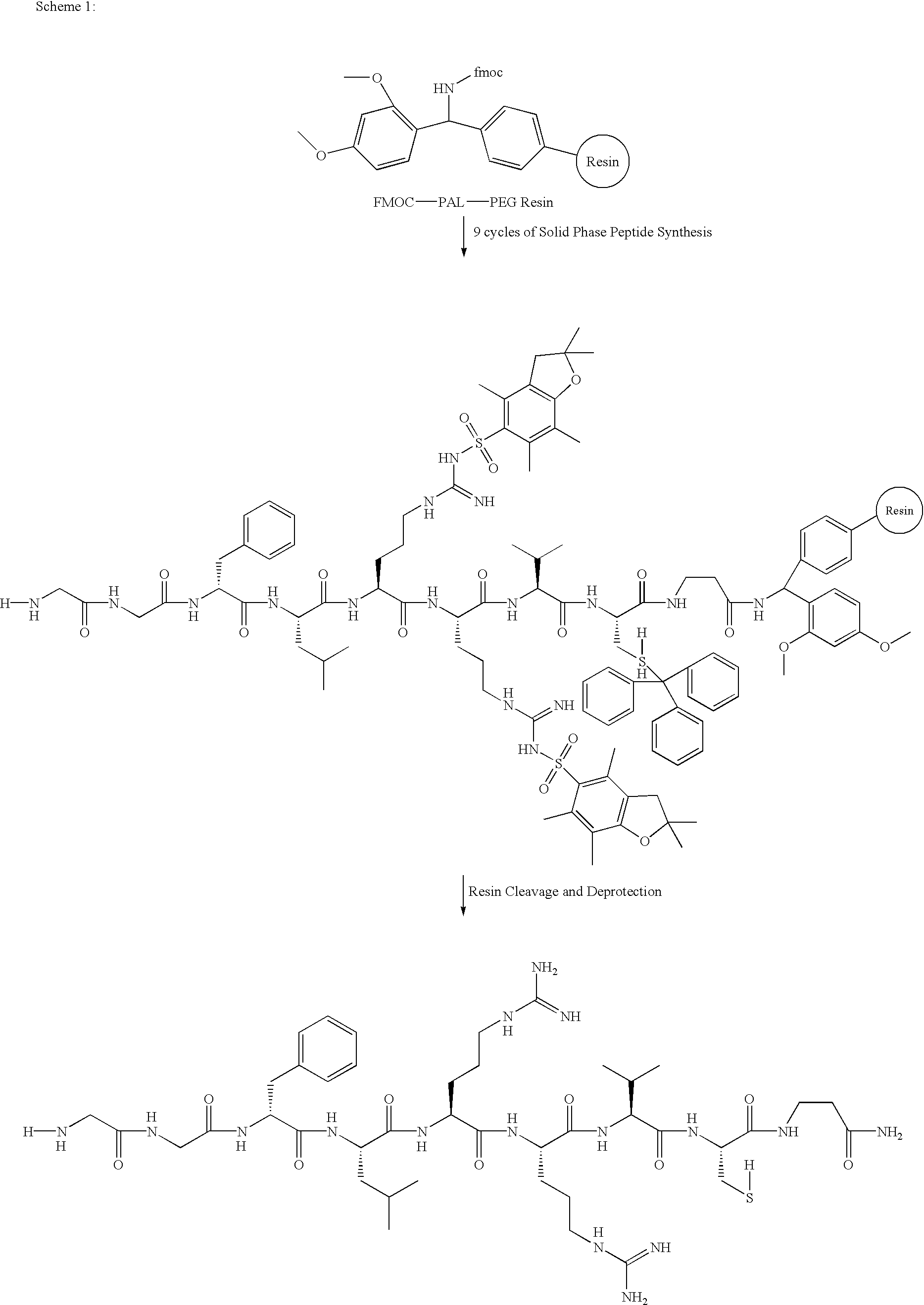

In summary, FMOC-PAL-PEG Resin is elaborated using Solid Phase Peptide Synthesis protocols optimised for efficiency of yield and time. These cycles (full details supra) incorporate 2 FMOC deprotections, washes, a single coupling of HBTU activated amino acid, washes, capping and finally, washing first with NMP then with 1:1 trifluoroethanol/dichloromethane. These washes help to relax resin secondary structure allowing for thorough deprotection and efficient coupling of the next incoming amino acid during the next cycle.

CP2 is synthesised (full details supra) according to Scheme 2:

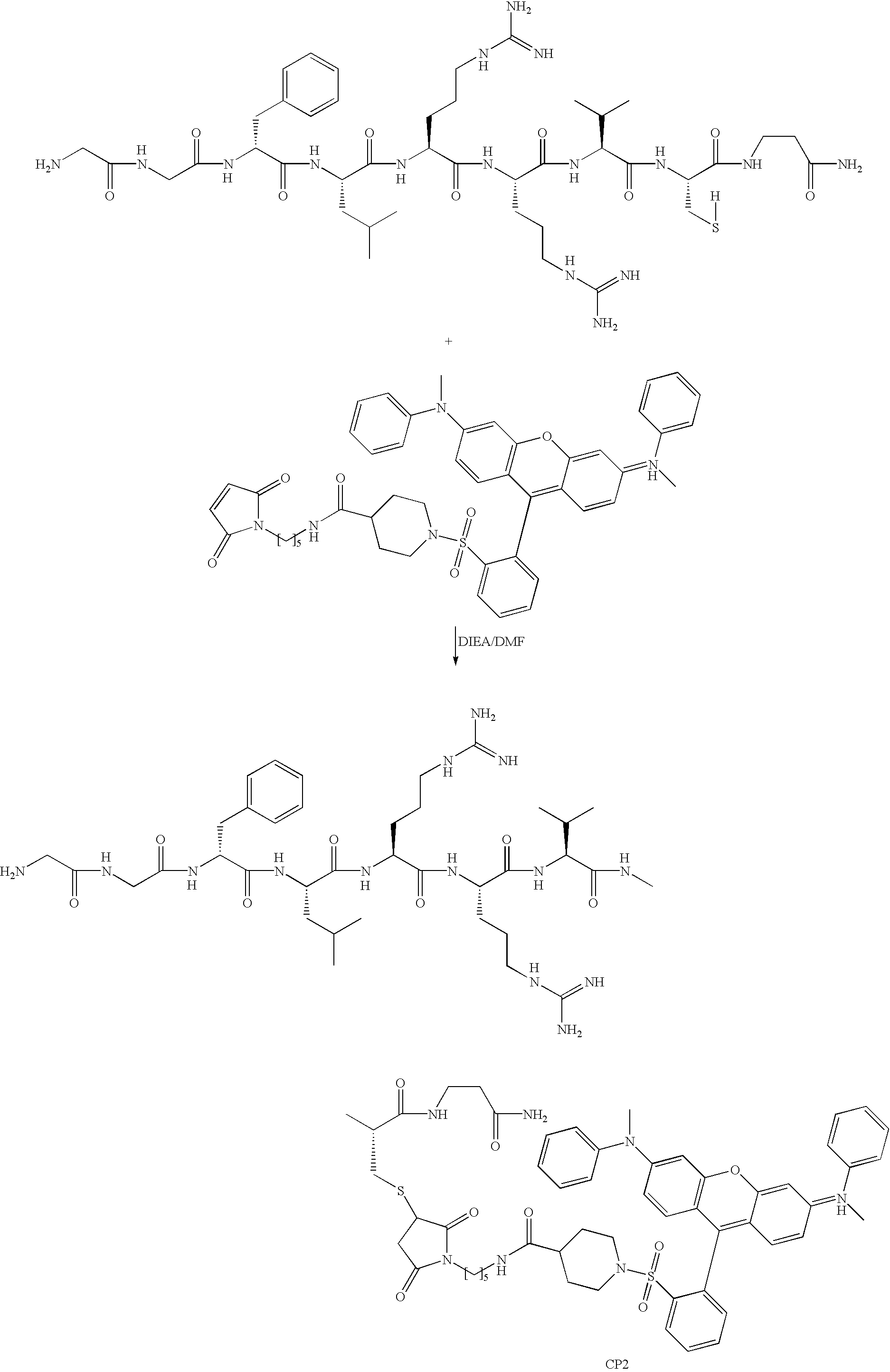

Following this incorporation of the QSY-7 tag, the second fluorophore, Rhodamine Green is added as the bis-trifluoroacetyl protected dye according to Scheme 3:

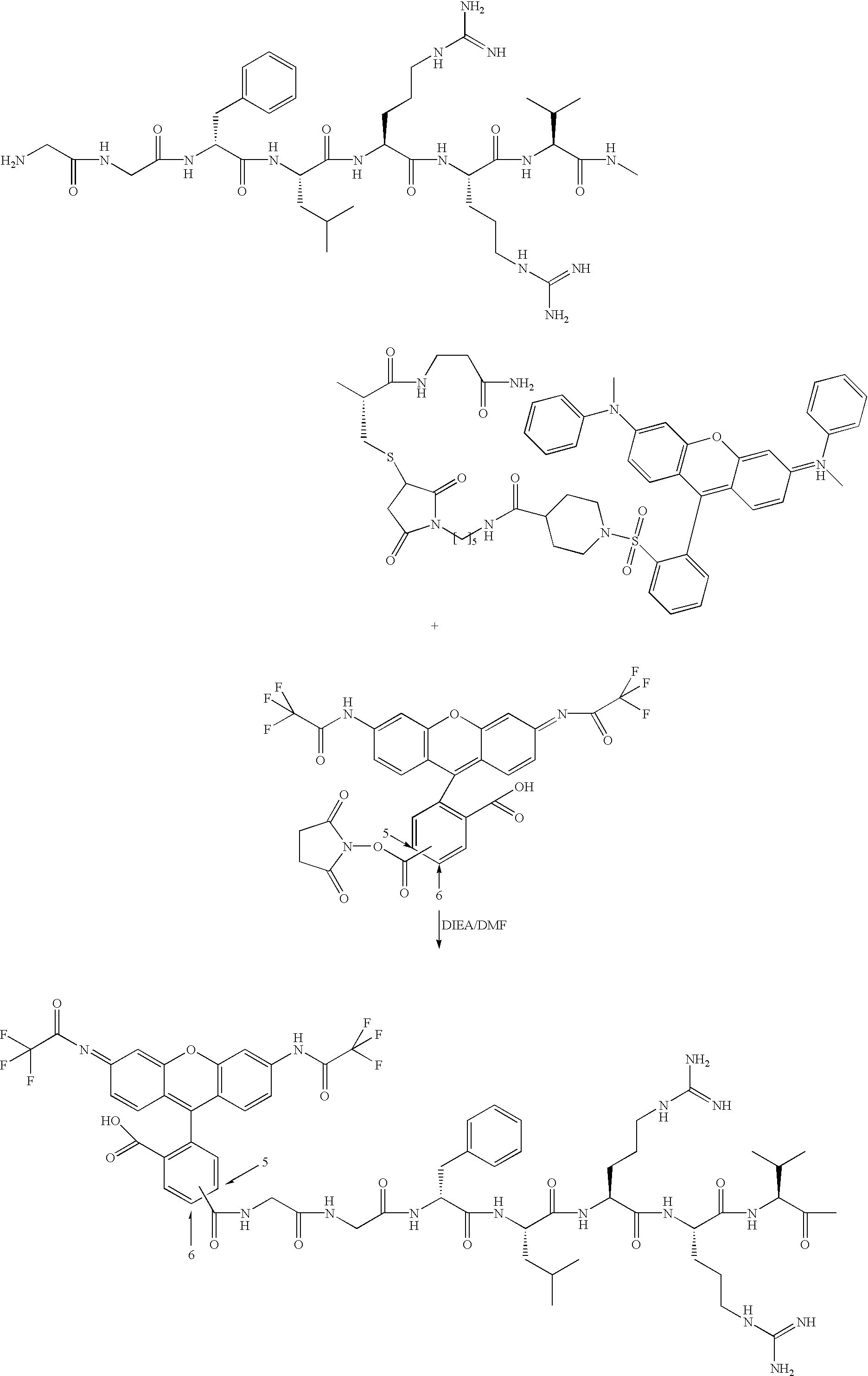

Finally, the trifluoroacetyl groups are removed by treatment with Na₂CO₃ affording the desired substrate, CP4:

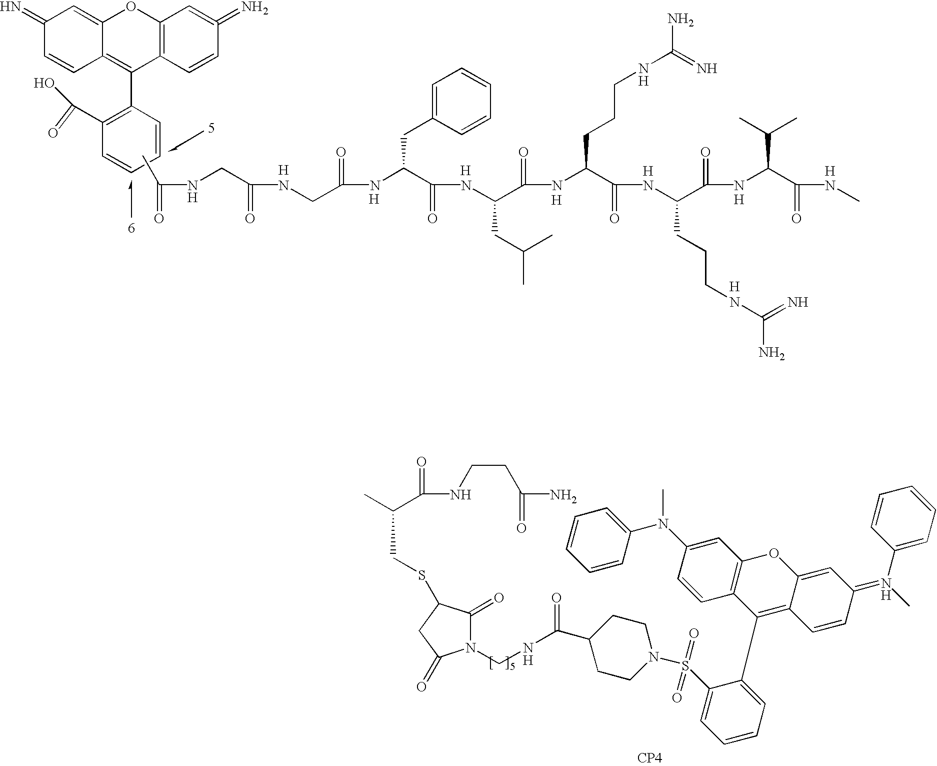

Assay

Reagents for the assay are first prepared as follows:

A substrate solution is made up by resuspending the substrate Rhodamine green-Gly-Gly-dPhe-Leu-Arg-Arg-Val-Cys(QSY7)-βAla-NH₂ in 50 mM HEPES buffer pH 7.4 (Sigma, UK) at a concentration of 2 μM, then adding 1 EDTA-free protease inhibitor cocktail tablet (Roche Diagnostics, UK) per 25 ml.

An aliquot of SEP enzyme described above is thawed then diluted in 50 mM HEPES, pH 7.4 by a predetermined factor specific to each enzyme batch, such that 50 μl contains sufficient enzyme to convert approximately 30% of substrate to product during the assay.

A 4% DMSO solution comprised of 4 ml DMSO plus 96 ml 50 mM HEPES pH 7.4 is prepared.

A product solution is prepared by adding 500 μl of substrate solution to 250 μl enzyme solution plus 250% of 4% DMSO solution, and incubating at 37° C. for 16 hours.

Assays are set up as follows:

In a black 96 well microtitre plate, 100 μl of substrate solution is added to 50 μl of 4% DMSO solution. A similar non-specific background blank is also set up in which the 50 μl of 4% DMSO solution additionally contains 40 μM phosphoramidon. 50 μlof enzyme solution is added to the assay and blank, and the 96 well plate placed in a BMG galaxy fluorescence reader, operating with the Biolise software package (BMG Lab technologies, Offenberg, Germany).

The plate is incubated in the fluorescence reader for 1 hour at 37° C. and a fluorescence measurement taken every 3 minutes (Excitation (Ex) 485 nm/Emission (Em) 535 nm). The proteolytic activity of SEP corresponds to the rate of increase in fluorescence of the sample—rate of increase in fluorescence units of the non-specific background blank. The maximum velocity measurement (MaxV) calculated by the software over four successive readings is used for this calculation.

A fluorescence measurement taken from 200 μl of product in a well on an identical microtitre plate is taken. If required this value is used, together with the measured fluorescence units from the 60 min timepoint of the SEP assay, to calculate the percentage (%) of the substrate proteolysed during the 1 hour incubation period or to convert the measured rates of fluorescence increase into other useful units such as ng substrate proteolysed/min/ml enzyme.

The assay is used to calculate enzyme kinetic parameters such as Vmax and Km following standard principles described in Fundamentals of Enzyme Kinetics by Athel Cornish Bowden, 1979, published by Butterworths.

Using the SEP Assay to Determine the Inhibition Parameters of SEP Inhibitors

To determine the IC₅₀ of SEP inhibitors (for example phosphoramidon), multiple SEP assays are performed as described above with a range of test concentrations of inhibitor included in the 50 μl of DMSO solution (made by appropriate dilution of a 10 mM 100% DMSO stock of inhibitor with 4% DMSO/50 mM HEPES pH 7.4.). Using a suitable standard graph fitting computer program, a sigmoidal dose response curve is fitted to a plot of log inhibitor concentration versus MaxV (or % inhibition or % activity). The IC₅₀ is calculated as the inhibitor concentration causing 50% maximal inhibition. Typically for a given IC₅₀ determination, a dose range of at least 10 inhibitor concentrations differing in half log unit increments is used.

The SEP assay is used to determine the Ki and mode of inhibition (i.e. whether the inhibition is competitive, mixed, non-competitive, etc.) following standard enzymology principles as described, for example, in Fundamentals of Enzyme Kinetics by Athel Cornish Bowden, 1979, published by Butterworths.

Auxiliary Agents—Modulators of Intermediate Conductance Calcium-Activated Potassium (IK_{Ca}) Channels

The term “calcium-activated potassium channels” includes large conductance calcium activated (BK_{Ca}) channels (also referred to as Maxi K+ channels), small conductance calcium activated (SK_{Ca}) channels and intermediate conductance calcium activated (IK_{Ca}) channels which are sometimes referred to as an hSK₄ channels or IK channels or hIK₁ channels.

Currently there are three subtypes of calcium-activated potassium channels. These are large conductance calcium activated (BK_{Ca}) channels, intermediate conductance calcium activated (IK_{Ca}) channels and small conductance calcium activated (SK_{Ca}) channels. These channels are characterised by the degree of ionic conductance that passes through the channel pore during a single opening (Fan et al 1995). By way of distinction: large conductance (BK) channels are gated by the concerted actions of internal calcium ions and membrane potential and have a unit conductance of 100 to 220 picoSiemens (pS); whereas Intermediate conductance (IK) and small conductance (SK) channels are gated solely by internal calcium ions. By way of further distinction, the IK_{Ca} and SK_{Ca} channels have a unit conductance of 20 to 85 pS and 2 to 20 pS, respectively, and are more sensitive to calcium than are BK channels. Each type of channel shows a distinct pharmacology (Ishii et al 1997).

As used herein, the term “intermediate conductance calcium activated (IK_{Ca}) channel” refers to a subtype of the calcium activated potassium channels which is characterised by the degree of ionic conductance that passes through the channel pore during a single opening (Fan et al 1995). In contrast to the large conductance (BK) channels which are gated by the concerted actions of internal calcium ions and membrane potential and have a unit conductance of 100 to 220 picoSiemens (pS), the intermediate conductance (IK) channel is gated solely by internal calcium ions, with a unit conductance of 20 to 85 pS and is more sensitive to calcium than the BK channels.

As used herein the term “modulating IK_{Ca} channel activity” means any one or more of: improving, increasing, enhancing, agonising, depolarising or upregulating IK_{Ca} channel activity or that the Ca²⁺ sensitivity of the IK_{Ca} channel is increased—that is, the calcium concentration required to elicit IK_{Ca} channel activity/opening is lowered. The increase in the Ca²⁺ sensitivity of the IK_{Ca} channel may be increased/enhanced by a direct or indirect opening of the IK_{Ca} channels. This increase in the Ca²⁺ sensitivity of the IK_{Ca} channel may result in a modification of the IK_{Ca} channel characteristics such that the IK_{Ca} channel opening is affected in such a way that the IK_{Ca} channel opens earlier and/or at lower intracellular calcium concentrations and/or for longer periods of time and/or with an increased open time probability.

The term “modulating IK_{Ca} channel activity” also includes the upregulation of IK_{Ca} channel expression in corpus cavernosum smooth muscle tissue such as, for example, by an agent that increases the expression of the IK_{Ca} channel and/or by the action of an agent on a substance that would otherwise impair and/or antagonise the modulation of IK_{Ca} channel activity and/or the expression of the IK_{Ca} channel.

By way of example the modulator may have the structure of formula (I):

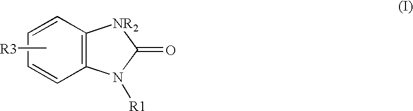

wherein

R1 is a H or a suitable substituent, such as an alkyl group which may be optionally substituted;

R2 is a H or a suitable substituent, preferably H

R3 represents one or more suitable optional substituents.

Alternatively, the modulator may have the structure of formula (1):

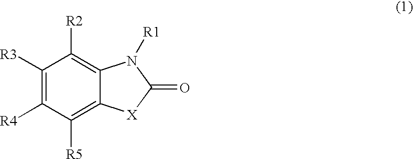

wherein:

X is selected from NR, O or S wherein R is H or alkyl (preferably lower alkyl, more preferably C1-6 alkyl)

R1 is alkyl (preferably lower alkyl, more preferably C1-6 alkyl)

R2 is selected from H, halide, alkyl (preferably lower alkyl, more preferably C1-6 alkyl), alkoxy (preferably lower alkoxy, more preferably C1-6 alkoxy)

R3 is selected from H, halide, alkyl (preferably lower alkyl, more preferably C1-6 alkyl), alkoxy (preferably lower alkoxy, more preferably C1-6 alkoxy)

R4 is selected from H, halide, alkyl (preferably lower alkyl, more preferably C1-6 alkyl), alkoxy (preferably lower alkoxy, more preferably C1-6 alkoxy)

R5 is selected from H, halide, alkyl (preferably lower alkyl, more preferably C1-6 alkyl), alkoxy (preferably lower alkoxy, more preferably C1-6 alkoxy).

Compounds of formula (1)—wherein X═O (formula (1a)) or wherein X═S (formula (1b))—can be prepared by N-alkylation under basic conditions of the respective corresponding parent heterocycles (2a) or (2b), these in turn may be prepared by the treatment of the respective corresponding aminophenol (3a) or aminothiophenol (3b) with phosgene or another suitable carbonylating agent. Aminophenols and aminothiophenols are usually prepared from the respective corresponding nitrophenols (4a) or nitrothiophenols (4b) by reduction. Many substituted nitrophenois (4a) and nitrothiophenols (4b) are commercially available.

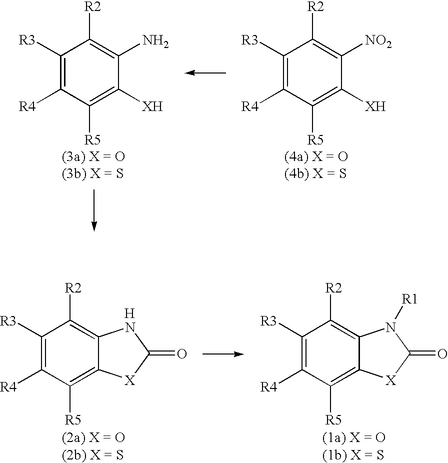

Compounds of formula I where X═NH (formula (1c)) can be prepared by a modification to the above scheme. In this respect, alkylation of a respective corresponding nitroaniline (5c) is carried out prior to reduction of the nitro group, providing a phenyldiamine (3c, X═NH) that is cyclised to 1 c by carbonylation as described above.

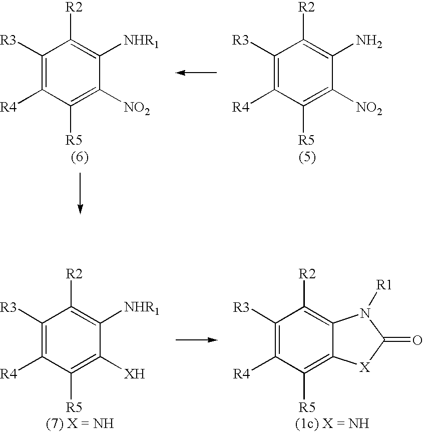

Preferably the modulator is EBIO (1-ethyl-2-benzimidazolinone) or a mimetic thereof or a pharmaceutically acceptable salt of any thereof. The structure of EBIO is:

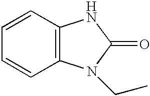

For some applications, preferably the agent has an IC₅₀ value of less than 300 nM, 250 nM, 200 nM, 150 nM, preferably less than about 100 nM, preferably less than about 75 nM, preferably less than about 50 nM, preferably less than about 25 nM, preferably less than about 20 nM, preferably less than about 15 nM, preferably less than about 10 nM, preferably less than about 5 nM.

For some applications, preferably the agent has at least about a 25, 50, 75, 100 fold selectivity to the desired target, preferably at least about a 150 fold selectivity to the desired target, preferably at least about a 200 fold selectivity to the desired target, preferably at least about a 250 fold selectivity to the desired target, preferably at least about a 300 fold selectivity to the desired target, preferably at least about a 350 fold selectivity to the desired target.

Corpus Cavernosum

As used herein, the term “corpus cavernosum” refers inter alia to a mass of tissue found in the penis. In this regard, the body of the penis is composed of three cylindrical masses of tissue, each surrounded by fibrous tissue called the tunica albuginea. The paired dorsolateral masses are called the corpora cavernosa penis (corpora=main bodies; cavernosa=hollow); the smaller midventral mass, the corpus spongiosum penis contains the spongy urethra and functions in keeping the spongy urethra open during ejaculation. All three masses are enclosed by fascia and skin and consist of erectile tissue permeated by blood sinuses. The corpus cavernosum comprises smooth muscle cells.

Treatment

It is to be appreciated that all references herein to treatment include one or more of curative, palliative and prophylactic treatment.

Sexual Stimulation

The present invention also encompasses use as defined hereinbefore via administration of an NPYi, preferably an NPY Y1i (and a PDEi, preferably a PDE5i, where applicable) before and/or during sexual stimulation. Here the term “sexual stimulation” may be synonymous with the term “sexual arousal”. This aspect of the present invention is advantageous because it provides systemic (physiological) selectivity. The natural cascade only occurs at the genitalia and not in other locations—e.g. in the heart etc. Hence, it is possible to achieve a selective effect on the genitalia via the MED treatment according to the present invention.

Thus, according to the present invention it is highly desirable that there is a sexual stimulation step at some stage. We have found that this step can provide systemic selectivity. Here, “sexual stimulation” may be one or more of a visual stimulation, a physical stimulation, an auditory stimulation, or a thought stimulation.

Agent

Agents for use in the treatment of male sexual dysfunction, in particular MED, according to the present invention may be any suitable agent that can act as an NPYi, preferably an NPY Y1i, and, where appropriate a combination of an NPYi, preferably an NPY Y1i, and a PDEi, preferably a PDE5i. As used herein, the term “agent” includes any entity capable of inhibiting NPY and/or NPY Y1 receptors.

Such agents (i.e. the agents as defined above) can be an amino acid sequence or a chemical derivative thereof. The substance may even be an organic compound or other chemical. The agent may even be a nucleotide sequence—which may be a sense sequence or an anti-sense sequence. The agent may even be an antibody.

Thus, the term “agent” includes, but is not limited to, a compound which may be obtainable from or produced by any suitable source, whether natural or not.

The agent may be designed or obtained from a library of compounds which may comprise peptides, as well as other compounds, such as small organic molecules, such as lead compounds.

By way of example, the agent may be a natural substance, a biological macromolecule, or an extract made from biological materials such as bacteria, fungi, or animal (particularly mammalian) cells or tissues, an organic or an inorganic molecule, a synthetic agent, a semi-synthetic agent, a structural or functional mimetic, a peptide, a peptidomimetics, a derivatised agent, a peptide cleaved from a whole protein, or a peptide synthesised synthetically (such as, by way of example, either using a peptide synthesiser or by recombinant techniques or combinations thereof, a recombinant agent, an antibody, a natural or a non-natural agent, a fusion protein or equivalent thereof and mutants, derivatives or combinations thereof.

An ACE assay is presented hereinbelow. For some applications (such as with particular individuals), such agents (i.e. those that also display ACE inhibitory action) may not be suitable for oral administration. Preferably, the NPY or NPY Y1 inhibitors according to the present invention has no, or substantially no, activity towards ACE.

ECE assays are well known in the art.

As used herein, the term “agent” may be a single entity or it may be a combination of agents.

If the agent is an organic compound then for some applications—such as if the agent is an NPYi or an NPY Y1i—that organic compound may typically comprise two or more linked hydrocarbyl groups. For some applications, preferably the agent comprises at least two cyclic groups—optionally wherein one of which cyclic groups may be a fused cyclic ring structure. For some applications, at least one of the cyclic groups is a heterocyclic group. For some applications, preferably the heterocyclic group comprises at least one N in the ring. Examples of such compounds are presented herein.

If the agent is an organic compound then for some applications—such as if the agent is an PDE5i—that organic compound may typically comprise two or more linked hydrocarbyl groups. For some applications, preferably the agent comprises at least two cyclic groups—wherein one of which cyclic groups may be a fused cyclic ring structure. For some applications, preferably at least one of the cyclic groups is a heterocyclic group. For some applications, preferably the heterocyclic group comprises at least one N in the ring. Examples of such compounds are presented in the PDE5 section herein.

The agent may contain halo groups. Here, “halo” means fluoro, chloro, bromo or iodo.

The agent may contain one or more of alkyl, alkoxy, alkenyl, alkylene and alkenylene groups—which may be unbranched- or branched-chain.

Pharmaceutically Acceptable Salt

The agent may be in the form of—and/or may be administered as—a pharmaceutically acceptable salt—such as an acid addition salt or a base salt—or a solvate thereof, including a hydrate thereof. For a review on suitable salts see Berge et al, J. Pharm. Sci., 1977, 66, 1-19.

Typically, a pharmaceutically acceptable salt may be readily prepared by using a desired acid or base, as appropriate. The salt may precipitate from solution and be collected by filtration or may be recovered by evaporation of the solvent.

Suitable acid addition salts are formed from acids which form non-toxic salts and examples are the hydrochloride, hydrobromide, hydroiodide, sulphate, bisulphate, nitrate, phosphate, hydrogen phosphate, acetate, maleate, fumarate, lactate, tartrate, citrate, gluconate, succinate, saccharate, benzoate, methanesulphonate, ethanesulphonate, benzenesulphonate, p-toluenesulphonate and pamoate salts.

Suitable base salts are formed from bases which form non-toxic salts and examples are the sodium, potassium, aluminium, calcium, magnesium, zinc and diethanolamine salts.

Polymorphic Form(s)/Asymmetric Carbon(s)

The agent may exist in polymorphic form.

The agent may contain one or more asymmetric carbon atoms and therefore exists in two or more stereoisomeric forms. Where an agent contains an alkenyl or alkenylene group, cis (E) and trans (Z) isomerism may also occur. The present invention includes the individual stereoisomers of the agent and, where appropriate, the individual tautomeric forms thereof, together with mixtures thereof.

Separation of diastereoisomers or cis and trans isomers may be achieved by conventional techniques, e.g. by fractional crystallisation, chromatography or H.P.L.C. of a stereoisomeric mixture of the agent or a suitable salt or derivative thereof. An individual enantiomer of the agent may also be prepared from a corresponding optically pure intermediate or by resolution, such as by H.P.L.C. of the corresponding racemate using a suitable chiral support or by fractional crystallisation of the diastereoisomeric salts formed by reaction of the corresponding racemate with a suitable optically active acid or base, as appropriate.

Isotopic Variations

The present invention also includes all suitable isotopic variations of the agent or a pharmaceutically acceptable salt thereof. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agent and pharmaceutically acceptable salts thereof can generally be prepared by conventional procedures using appropriate isotopic variations of suitable reagents.

Prodrugs

It will be appreciated by those skilled in the art that the agent may be derived from a prodrug. Examples of prodrugs include entities that have certain protected group(s) and which may not possess pharmacological activity as such, but may, in certain instances, be administered (such as orally or parenterally) and thereafter metabolised in the body to form the agent which are pharmacologically active.

Pro-Moieties

It will be further appreciated that certain moieties known as “pro-moieties”, for example as described in “Design of Prodrugs” by H. Bundgaard, Elsevier, 1985 (the disclosure of which is hereby incorporated by reference), may be placed on appropriate functionalities of the agents. Such prodrugs are also included within the scope of the invention.

Inhibitor/Antagonist

The term inhibitor as used herein in relation to the NPYi or NPY Y1i (and where applicable PDEi or PDE5i compounds and other auxiliary active agents) is to be regarded as being interchangeable with the term antagonist.

As used herein, the term “antagonist” means any agent that reduces the action of another agent or target. The antagonistic action may result form a combination of the substance being antagonised (chemical antagonism) or the production of an opposite effect through a different target (functional antagonism or physiological antagonism) or as a consequence of competition for the binding site of an intermediate that links target activation to the effect observed (indirect antagonism).

Further the phrase, enhancing the endogenous erectile process, is to be regarded as being interchangeable with the phrase upregulation of the endogenous erectile process.

Pharmaceutical Compositions

The present invention also provides a pharmaceutical composition comprising a therapeutically effective amount of the agent of the present invention and a pharmaceutically acceptable carrier, diluent or excipient (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilisers, dyes and even flavouring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be delivered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be delivered by both routes.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

For some embodiments, the agents of the present invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

In a preferred embodiment, the agents of the present invention are delivered systemically (such as orally, buccally, sublingually), more preferably orally.

Hence, preferably the agent is in a form that is suitable for oral delivery.

For some embodiments, preferably the agent—when in use—in addition to acting peripherally on the genitalia the agent also acts on the central nervous system.

For some embodiments, preferably the agent—when in use—is not peripherally acting other than in respect of receptors located in the genitalia and preferably those associated with the corpus cavernosum.

Administration

The term “administered” includes delivery by viral or non-viral techniques. Viral delivery mechanisms include but are not limited to adenoviral vectors, adeno-associated viral (AAV) vectors, herpes viral vectors, retroviral vectors, lentiviral vectors, and baculoviral vectors. Non-viral delivery mechanisms include lipid mediated transfection, liposomes, immunoliposomes, lipofectin, cationic facial amphiphiles (CFAs) and combinations thereof.

The agents of the present invention may be administered alone but will generally be administered as a pharmaceutical composition—e.g. when the agent is in admixture with a suitable pharmaceutical excipient, diluent or carrier selected with regard to the intended route of administration and standard pharmaceutical practice.

For example, the agent can be administered (e.g. orally or topically) in the form of tablets, capsules, ovules, elixirs, solutions or suspensions, which may contain flavouring or colouring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

The routes for administration (delivery) include, but are not limited to, one or more of: oral (e.g. as a tablet, capsule, or as an ingestable solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, penile, vaginal, epidural, sublingual.

It is to be understood that not all of the agents need be administered by the same route. Likewise, if the composition comprises more than one active component, then those components may be administered by different routes.

If the agent of the present invention is administered parenterally, then examples of such administration include one or more of: intravenously, intra-arterially, intraperitoneally, intrathecally, intraventricularly, intraurethrally, intrasternally, intracranially, intramuscularly or subcutaneously administering the agent; and/or by using infusion techniques.

For parenteral administration, the agent is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

As indicated, the agent of the present invention can be administered intranasally or by inhalation and is conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 1₃₄A™) or 1,1,1,2,3,3,3-heptafluoropropane (HFA 227EA™), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the agent and a suitable powder base such as lactose or starch.

Alternatively, the agent of the present invention can be administered in the form of a suppository or pessary, or it may be applied topically in the form of a gel, hydrogel, lotion, solution, cream, ointment or dusting powder. The agent of the present invention may also be dermally or transdermally administered, for example, by the use of a skin patch. They may also be administered by the pulmonary or rectal routes. They may also be administered by the ocular route. For ophthalmic use, the compounds can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the agent of the present invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, it can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

The compositions of the present invention may be administered by direct injection.

For some applications, preferably the agent is administered orally.

For some applications, preferably the agent is administered topically.

Dose Levels

Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The agent and/or the pharmaceutical composition of the present invention may be administered in accordance with a regimen of from 1 to 10 times per day, such as once or twice per day.

For oral and parenteral administration to humans, the daily dosage level of the agent may be in single or divided doses.

Depending upon the need, the agent may be administered at a dose of from 0.01 to 30 mg/kg body weight, such as from 0.1 to 10 mg/kg, more preferably from 0.1 to 1 mg/kg body weight. Naturally, the dosages mentioned herein are exemplary of the average case. There can, of course, be individual instances where higher or lower dosage ranges are merited.

Typically the daily oral dose may be, for instance, between 20-1000 mg, preferably 50-300 mg for example.

Formulation

The agents of the present invention may be formulated into a pharmaceutical composition, such as by mixing with one or more of a suitable carrier, diluent or excipient, by using techniques that are known in the art.

The following present some non-limiting examples of formulations.

Formulation 1: A tablet is prepared using the following ingredients:

| | weight/mg |
|---|---|
| Agent | 250 |
| Cellulose, microcrystalline | 400 |
| Silicon dioxide, fumed | 10 |
| Stearic acid | 5 |
| Total | 665 |

the components are blended and compressed to form tablets each weighing 665 mg.

Formulation 2: An intravenous formulation may be prepared as follows:

| | |
|---|---|
| Agent | 100 mg |
| Isotonic saline | 1,000 ml |

Individual

As used herein, the term “individual” refers to vertebrates, particularly members of the mammalian species. The term includes but is not limited to domestic animals, sports animals, primates and humans.

Bioavailability

Preferably, the compounds of the invention (and combinations) are orally bioavailable. Oral bioavailablity refers to the proportion of an orally administered drug that reaches the systemic circulation. The factors that determine oral bioavailability of a drug are dissolution, membrane permeability and metabolic stability. Typically, a screening cascade of firstly in vitro and then in vivo techniques is used to determine oral bioavailablity.

Dissolution, the solubilisation of the drug by the aqueous contents of the gastrointestinal tract (GIT), can be predicted from in vitro solubility experiments conducted at appropriate pH to mimic the GIT. Preferably the compounds of the invention have a minimum solubility of 50 mcg/ml. Solubility can be determined by standard procedures known in the art such as described in Adv. Drug Deliv. Rev. 23, 3-25, 1997.

Membrane permeability refers to the passage of the compound through the cells of the GIT. Lipophilicity is a key property in predicting this and is defined by in vitro Log D_{7.4} measurements using organic solvents and buffer. Preferably the compounds of the invention have a Log D_{7.4} of −2 to +4, more preferably −1 to +2. The log D can be determined by standard procedures known in the art such as described in J. Pharm. Pharmacol. 1990, 42:144.

Cell monolayer assays such as CaCO₂ add substantially to prediction of favourable membrane permeability in the presence of efflux transporters such as p-glycoprotein, so-called caco-2 flux. Preferably, compounds of the invention have a caco-2 flux of greater than 2×10⁻⁶cms⁻¹, more preferably greater than 5×10⁻⁶ cms¹. The caco flux value can be determined by standard procedures known in the art such as described in J. Pharm. Sci, 1990, 79, 595-600

Metabolic stability addresses the ability of the GIT or the liver to metabolise compounds during the absorption process: the first pass effect. Assay systems such as microsomes, hepatocytes etc are predictive of metabolic liability. Preferably the compounds of the Examples show metabolic stability in the assay system that is commensurate with an hepatic extraction of less then 0.5. Examples of assay systems and data manipulation are described in Curr. Opin. Drug Disc. Devel., 201, 4, 36-44, Drug Met. Disp.,2000, 28, 1518-1523.

Because of the interplay of the above processes further support that a drug will be orally bioavailable in humans can be gained by in vivo experiments in animals. Absolute bioavailability is determined in these studies by administering the compound separately or in mixtures by the oral route. For absolute determinations (% absorbed) the intravenous route is also employed. Examples of the assessment of oral bioavailability in animals can be found in Drug Met. Disp.,2001, 29, 82-87; J. Med Chem, 1997, 40, 827-829, Drug Met. Disp.,1999, 27, 221-226.

Chemical Synthesis Methods

Typically the NPYi/NPY Y1i (and/or PDEi/PDE5i where applicable) suitable for the use according to the present invention will be prepared by chemical synthesis techniques.

The agent or target or variants, homologues, derivatives, fragments or mimetics thereof may be produced using chemical methods to synthesise the agent in whole or in part. For example, peptides can be synthesised by solid phase techniques, cleaved from the resin, and purified by preparative high performance liquid chromatography (e.g., Creighton (1983) Proteins Structures And Molecular Principles, W H Freeman and Co, New York N.Y.). The composition of the synthetic peptides may be confirmed by amino acid analysis or sequencing (e.g., the Edman degradation procedure; Creighton, supra).

Direct synthesis of the agent or variants, homologues, derivatives, fragments or mimetics thereof can be performed using various solid-phase techniques (Roberge J Y et al (1995) Science 269: 202-204) and automated synthesis may be achieved, for example, using the ABI 43 1 A Peptide Synthesizer (Perkin Elmer) in accordance with the instructions provided by the manufacturer. Additionally, the amino acid sequences comprising the agent or any part thereof, may be altered during direct synthesis and/or combined using chemical methods with a sequence from other subunits, or any part thereof, to produce a variant agent or target, such as, for example, a variant NPY or NPY Y1.

In an alternative embodiment of the invention, the coding sequence of the agent target or variants, homologues, derivatives, fragments or mimetics thereof may be synthesised, in whole or in part, using chemical methods well known in the art (see Caruthers M H et al (1980) Nuc Acids Res Symp Ser 215-23, Horn T et al (1980) Nuc Acids Res Symp Ser 225-232).

Mimetic

As used herein, the term “mimetic” relates to any chemical which includes, but is not limited to, a peptide, polypeptide, antibody or other organic chemical which has the same qualitative activity or effect as a reference agent to a target. That is a mimetic may be a functional equivalent to a known agent.

Chemical Derivative

The term “derivative” or “derivatised” as used herein includes chemical modification of an agent. Illustrative of such chemical modifications would be replacement of hydrogen by a halo group, an alkyl group, an acyl group or an amino group.

Chemical Modification

In one embodiment of the present invention, the agent may be a chemically modified agent.

The chemical modification of an agent may either enhance or reduce hydrogen bonding interaction, charge interaction, hydrophobic interaction, Van Der Waals interaction or dipole interaction between the agent and the target.

In one aspect, the identified agent may act as a model (for example, a template) for the development of other compounds.

Targets

In one aspect of the present invention, an NPY or NPY Y1 receptor may be used as a target in screens to identify agents capable of inhibiting NPY or NPY Y1. In this regard, the target may comprise an amino acid sequence encoded by the nucleotide sequences shown as SEQ ID No. 1, SEQ ID No. 2 or SEQ ID No. 3 or a variant, homologue, derivative or fragment thereof which is prepared by recombinant and/or synthetic means or an expression entity comprising same.

Alternatively, an NPY or NPY Y1 receptor may be used to as a target to identify agents capable of mediating an increase in intracavernosal pressure through the inhibition of NPY or NPY Y1. In this respect, the target may be suitable tissue extract.

The target may even be a combination of such tissue and/or recombinant targets.

Recombinant Methods

Typically the agent of the present invention may be prepared by recombinant DNA techniques.

In one embodiment, preferably the agent is an NPYi or an NPY Y1i. The NPYi or the NPY Y1i may be prepared by recombinant DNA techniques.

Amino Acid Sequence

As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “protein”.

The amino acid sequence may be prepared isolated from a suitable source, or it may be made synthetically or it may be prepared by use of recombinant DNA techniques.

In one aspect, the present invention provides an amino acid sequence that is capable of acting as a target in an assay for the identification of one or more agents and/or derivatives thereof.

Preferably, the target is an NPY or NPY Y1 receptor.

Preferably, the NPY or NPY Y1 receptor is an isolated NPY or NPY Y1 receptor and/or is purified and/or is non-native.

The NPY or NPY Y1 receptor of the present invention may be in a substantially isolated form. It will be understood that the NPY or NPY Y1 receptor may be mixed with carriers or diluents which will not interfere with the intended purpose of the receptor and which will still be regarded as substantially isolated. The NPY or NPY Y1 receptor of the present invention may also be in a substantially pure form, in which case it will generally comprise the NPY or NPY Y1 receptor in a preparation in which more than 90%, e.g. 95%, 98% or 99% of the NPY or NPY Y1 receptor in the preparation is a peptide obtainable from the expression of SEQ ID No. 1, 2 or 3 or variants, homologues, derivative or fragments thereof.

Nucleotide Sequence

As used herein, the term “nucleotide sequence” is synonymous with the term “polynucleotide”.

The nucleotide sequence may be DNA or RNA of genomic or synthetic or of recombinant origin. The nucleotide sequence may be double-stranded or single-stranded whether representing the sense or antisense strand or combinations thereof.

For some applications, preferably, the nucleotide sequence is DNA.

For some applications, preferably, the nucleotide sequence is prepared by use of recombinant DNA techniques (e.g. recombinant DNA).

For some applications, preferably, the nucleotide sequence is cDNA.

For some applications, preferably, the nucleotide sequence may be the same as the naturally occurring form for this aspect.

In one aspect, the present invention provides a nucleotide sequence encoding a substance capable of acting as a target in an assay for the identification of one ore more agents and/or derivative thereof.

In one aspect of the present invention the nucleotide sequence encodes an NPY or an NPY Y1 receptor.

It will be understood by a skilled person that numerous different nucleotide sequences can encode the same target as a result of the degeneracy of the genetic code. In addition, it is to be understood that skilled persons may, using routine techniques, make nucleotide substitutions that do not substantially affect the activity encoded by the nucleotide sequence of the present invention to reflect the codon usage of any particular host organism in which the target is to be expressed. Thus, the terms “variant”, “homologue” or “derivative” in relation to the nucleotide sequence set out in the attached sequence listings include any substitution of, variation of, modification of, replacement of, deletion of or addition of one (or more) nucleic acid from or to the sequence providing the resultant nucleotide sequence encodes a functional target according the present invention (or even an agent according to the present invention if said agent comprises a nucleotide sequence or an amino acid sequence).

As indicated above, with respect to sequence homology, preferably there is at least 75%, more preferably at least 85%, more preferably at least 90% homology to the NPY sequence cross referenced to herein. More preferably there is at least 95%, more preferably at least 98%, homology. Nucleotide homology comparisons may be conducted as described above. A preferred sequence comparison program is the GCG Wisconsin Bestfit program described above. The default scoring matrix has a match value of 10 for each identical nucleotide and −9 for each mismatch. The default gap creation penalty is −50 and the default gap extension penalty is −3 for each nucleotide.

The present invention also encompasses nucleotide sequences that are capable of hybridising selectively to the sequences presented herein, or any variant, fragment or derivative thereof, or to the complement of any of the above. Nucleotide sequences are preferably at least 15 nucleotides in length, more preferably at least 20, 30, 40 or 50 nucleotides in length. These sequences could be used a probes, such as in a diagnostic kit.

Variants/Homologues/Derivatives

In addition to the specific nucleotide sequences mentioned herein and amino acid sequences derivable therefrom, the present invention also encompasses the use of variants, homologue and derivatives thereof. Here, the term “homology” can be equated with “identity”.

In the present context, an homologous sequence is taken to include an amino acid sequence which may be at least 75, 85 or 90% identical, preferably at least 95 or 98% identical. In particular, homology should typically be considered with respect to those regions of the sequence known to be essential for an activity. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However it is preferred to use the GCG Bestfit program. A new tool, called BLAST 2 Sequences is also available for comparing protein and nucleotide sequence (see FEMS Microbiol Lett 1999 174(2): 247-50; FEMS Microbiol Lett 1999 177(1): 187-8 and tatiana@ncbi.nim.nih.gov).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

The sequences may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | |
|---|---|
| ALIPHATIC | |
| Non-polar | GAP |
| | ILV |
| Polar - uncharged | CSTM |
| | NQ |
| Polar - charged | DE |
| | KR |
| AROMATIC | HFWY |

The present invention also encompasses homologous substitution (substitution and replacement are both used herein to mean the interchange of an existing amino acid residue, with an alternative residue) may occur i.e. like-for-like substitution such as basic for basic, acidic for acidic, polar for polar etc. Non-homologous substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

Replacements may also be made by unnatural amino acids include; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, β-alanine*, L-α-amino butyric acid*, L-γ-amino butyric acid*, L-α-amino isobutyric acid*, L-ε-amino caproic acid^{#}, 7-amino heptanoic acid*, L-methionine sulfone^{#*}, L-norleucine*, L-norvaline*, p-nitro-L-phenylalanine*, L-hydroxyproline^{#}, L-thioproline*, methyl derivatives of phenylalanine (Phe) such as 4-methyl-Phe*, pentamethyl-Phe*, L-Phe (4-amino)^{#}, L-Tyr (methyl)*, L-Phe (4-isopropyl)*, L-Tic (1,2,3,4-tetrahydroisoquinoline-3-carboxyl acid)*, L-diaminopropionic acid^{#} and L-Phe (4-benzyl)*. The notation * has been utilised for the purpose of the discussion above (relating to homologous or non-homologous substitution), to indicate the hydrophobic nature of the derivative whereas # has been utilised to indicate the hydrophilic nature of the derivative, #* indicates amphipathic characteristics.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Horwell D C, Trends Biotechnol. (1995) 13(4), 132-134.

Hybridisation

The term “hybridisation” as used herein shall include “the process by which a strand of nucleic acid joins with a complementary strand through base pairing” as well as the process of amplification as carried out in polymerase chain reaction (PCR) technologies.

Nucleotide sequences of the invention capable of selectively hybridising to the nucleotide sequences presented herein, or to their complement, will be generally at least 75%, preferably at least 85 or 90% and more preferably at least 95% or 98% homologous to the corresponding complementary nucleotide sequences presented herein over a region of at least 20, preferably at least 25 or 30, for instance at least 40, 60 or 100 or more contiguous nucleotides.

The term “selectively hybridizable” means that the nucleotide sequence, when used as a probe, is used under conditions where a target nucleotide sequence is found to hybridise to the probe at a level significantly above background. The background hybridisation may occur because of other nucleotide sequences present, for example, in the cDNA or genomic DNA library being screened. In this event, background implies a level of signal generated by interaction between the probe and a non-specific DNA member of the library which is less than 10 fold, preferably less than 100 fold as intense as the specific interaction observed with the target DNA. The intensity of interaction may be measured, for example, by radiolabelling the probe, e.g. with ³²P.

Hybridisation conditions are based on the melting temperature (Tm) of the nucleic acid binding complex, as taught in Berger and Kimmel (1987, Guide to Molecular Cloning Techniques, Methods in Enzymology, Vol. 152, Academic Press, San Diego Calif.), and confer a defined “stringency” as explained below.

Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridisation can be used to identify or detect identical nucleotide sequences while an intermediate (or low) stringency hybridisation can be used to identify or detect similar or related polynucleotide sequences.

In a preferred aspect, the present invention covers nucleotide sequences that can hybridise to the nucleotide sequence of the present invention under stringent conditions (e.g. 65° C. and 0.1× SSC {1× SSC=0.15 M NaCl, 0.015 M Na₃Citrate pH 7.0). Where the nucleotide sequence of the invention is double-stranded, both strands of the duplex, either individually or in combination, are encompassed by the present invention. Where the nucleotide sequence is single-stranded, it is to be understood that the complementary sequence of that nucleotide sequence is also included within the scope of the present invention.

Nucleotide sequences which are not 100% homologous to the sequences of the present invention but fall within the scope of the invention can be obtained in a number of ways. Other variants of the sequences described herein may be obtained for example by probing DNA libraries made from a range of sources. In addition, other viral/bacterial, or cellular homologues particularly cellular homologues found in mammalian cells (e.g. rat, mouse, bovine and primate cells), may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of the nucleotide sequence set out in herein under conditions of medium to high stringency. Similar considerations apply to obtaining species homologues and allelic variants of the amino acid and/or nucleotide sequences of the present invention.

Variants and strain/species homologues may also be obtained using degenerate PCR which will use primers designed to target sequences within the variants and homologues encoding conserved amino acid sequences within the sequences of the present invention. Conserved sequences can be predicted, for example, by aligning the amino acid sequences from several variants/homologues. Sequence alignments can be performed using computer software known in the art. For example the GCG Wisconsin PileUp program is widely used. The primers used in degenerate PCR will contain one or more degenerate positions and will be used at stringency conditions lower than those used for cloning sequences with single sequence primers against known sequences.

Alternatively, such nucleotide sequences may be obtained by site directed mutagenesis of characterised sequences, such as the nucleotide sequence set out in SEQ ID Nos 1 or 2 of the sequence listings of the present invention. This may be useful where for example silent codon changes are required to sequences to optimise codon preferences for a particular host cell in which the nucleotide sequences are being expressed. Other sequence changes may be desired in order to introduce restriction enzyme recognition sites, or to alter the activity of the protein encoded by the nucleotide sequences.

The nucleotide sequences of the present invention may be used to produce a primer, e.g. a PCR primer, a primer for an alternative amplification reaction, a probe e.g. labelled with a revealing label by conventional means using radioactive or non-radioactive labels, or the nucleotide sequences may be cloned into vectors. Such primers, probes and other fragments will be at least 15, preferably at least 20, for example at least 25, 30 or 40 nucleotides in length, and are also encompassed by the term nucleotide sequence of the invention as used herein.

The nucleotide sequences such as a DNA polynucleotides and probes according to the invention may be produced recombinantly, synthetically, or by any means available to those of skill in the art. They may also be cloned by standard techniques.

In general, primers will be produced by synthetic means, involving a step wise manufacture of the desired nucleic acid sequence one nucleotide at a time. Techniques for accomplishing this using automated techniques are readily available in the art.

Longer nucleotide sequences will generally be produced using recombinant means, for example using a PCR (polymerase chain reaction) cloning techniques. This will involve making a pair of primers (e.g. of about 15 to 30 nucleotides) flanking a region of the targeting sequence which it is desired to clone, bringing the primers into contact with mRNA or cDNA obtained from an animal or human cell, performing a polymerase chain reaction (PCR) under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector.

Due to the inherent degeneracy of the genetic code, other DNA sequences which encode substantially the same or a functionally equivalent amino acid sequence, may be used to clone and express the target sequences. As will be understood by those of skill in the art, for certain expression systems, it may be advantageous to produce the target sequences with non-naturally occurring codons. Codons preferred by a particular prokaryotic or eukaryotic host (Murray E et al (1989) Nuc Acids Res 17:477-508) can be selected, for example, to increase the rate of the target expression or to produce recombinant RNA transcripts having desirable properties, such as a longer half-life, than transcripts produced from naturally occurring sequence.

Vector

In one embodiment of the present invention, an agent (i.e. an NPYi or an NPY Y1i) may be administered directly to an individual.

In another embodiment of the present invention, a vector comprising a nucleotide sequence encoding an agent of the present invention is administered to an individual.

Preferably the recombinant agent is prepared and/or delivered to a target site using a genetic vector.

As it is well known in the art, a vector is a tool that allows or facilitates the transfer of an entity from one environment to another. In accordance with the present invention, and by way of example, some vectors used in recombinant DNA techniques allow entities, such as a segment of DNA (such as a heterologous DNA segment, such as a heterologous cDNA segment), to be transferred into a host and/or a target cell for the purpose of replicating the vectors comprising the nucleotide sequences of the present invention and/or expressing the proteins of the invention encoded by the nucleotide sequences of the present invention. Examples of vectors used in recombinant DNA techniques include but are not limited to plasmids, chromosomes, artificial chromosomes or viruses.

The term “vector” includes expression vectors and/or transformation vectors.

The term “expression vector” means a construct capable of in vivo or in vitro/ex vivo expression.

The term “transformation vector” means a construct capable of being transferred from one species to another.

Naked DNA

The vectors comprising nucleotide sequences encoding an agent of the present invention for use in treating MSDs such as MED may be administered directly as “a naked nucleic acid construct”, preferably further comprising flanking sequences homologous to the host cell genome.

As used herein, the term “naked DNA” refers to a plasmid comprising a nucleotide sequences encoding an agent of the present invention together with a short promoter region to control its production. It is called “naked” DNA because the plasmids are not carried in any delivery vehicle. When such a DNA plasmid enters a host cell, such as a eukaryotic cell, the proteins it encodes (such as an agent of the present invention) are transcribed and translated within the cell.

Non-Viral Delivery

Alternatively, the vectors comprising nucleotide sequences of the present invention or an agent of the present invention (i.e. NPYi or NPY Y1i) or a target of the present invention (i.e. NPY or NPY Y1) may be introduced into suitable host cells using a variety of non-viral techniques known in the art, such as transfection, transformation, electroporation and biolistic transformation.

As used herein, the term “transfection” refers to a process using a non-viral vector to deliver a gene to a target mammalian cell.

Typical transfection methods include electroporation, DNA biolistics, lipid-mediated transfection, compacted DNA-mediated transfection, liposomes, immunoliposomes, lipofectin, cationic agent-mediated, cationic facial amphiphiles (CFAS) (Nature Biotechnology 1996 14; 556), multivalent cations such as spermine, cationic lipids or polylysine, 1, 2,-bis (oleoyloxy)-3-(trimethylammonio) propane (DOTAP)-cholesterol complexes (Wolff and Trubetskoy 1998 Nature Biotechnology 16: 421) and combinations thereof.

Uptake of naked nucleic acid constructs by mammalian cells is enhanced by several known transfection techniques for example those including the use of transfection agents. Example of these agents include cationic agents (for example calcium phosphate and DEAE-dextran) and lipofectants (for example lipofectam™ and transfectam™). Typically, nucleic acid constructs are mixed with the transfection agent to produce a composition.

Viral Vectors

Alternatively, the vectors comprising an agent or target of the present invention or nucleotide sequences of the present invention may be introduced into suitable host cells using a variety of viral techniques which are known in the art, such as for example infection with recombinant viral vectors such as retroviruses, herpes simplex viruses and adenoviruses.

Preferably the vector is a recombinant viral vectors. Suitable recombinant viral vectors include but are not limited to adenovirus vectors, adeno-associated viral (AAV) vectors, herpes-virus vectors, a retroviral vector, lentiviral vectors, baculoviral vectors, pox viral vectors or parvovirus vectors (see Kestler et al 1999 Human Gene Ther 10(10):1619-32). In the case of viral vectors, delivery of the nucleotide sequence encoding the agent of the present invention is mediated by viral infection of a target cell.

Targeted Vector

The term “targeted vector” refers to a vector whose ability to infect/transfect/transduce a cell or to be expressed in a host and/or target cell is restricted to certain cell types within the host organism, usually cells having a common or similar phenotype.

Replication Vectors

The nucleotide sequences encoding an agent (i.e. NPYi or NPY Y1i or PDEi or PDE5i) of the present invention or a target (such as NPY or NPY Y1) may be incorporated into a recombinant replicable vector. The vector may be used to replicate the nucleotide sequence in a compatible host cell. Thus in one embodiment of the present invention, the invention provides a method of making a target of the present invention by introducing a nucleotide sequence of the present invention into a replicable vector, introducing the vector into a compatible host cell, and growing the host cell under conditions which bring about replication of the vector. The vector may be recovered from the host cell.

Expression Vector

Preferably, an agent of the present invention or a nucleotide sequence of present invention or a target of the present invention which is inserted into a vector is operably linked to a control sequence that is capable of providing for the expression of the coding sequence, such as the coding sequence of the NPY or NPY Y1 of the present invention by the host cell, i.e. the vector is an expression vector. An agent of the present invention or a target produced by a host recombinant cell may be secreted or may be contained intracellularly depending on the sequence and/or the vector used. As will be understood by those of skill in the art, expression vectors containing an agent or target of the present invention coding sequences can be designed with signal sequences which direct secretion of the agent or target of the present invention coding sequences through a particular prokaryotic or eukaryotic cell membrane.

Expression in vitro

The vectors of the present invention may be transformed or transfected into a suitable host cell and/or a target cell as described below to provide for expression of an agent or a target of the present invention. This process may comprise culturing a host cell and/or target cell transformed with an expression vector under conditions to provide for expression by the vector of a coding sequence encoding an agent or a target of the present invention and optionally recovering the expressed agent or target of the present invention. The vectors may be for example, plasmid or virus vectors provided with an origin of replication, optionally a promoter for the expression of the said polynucleotide and optionally a regulator of the promoter. The vectors may contain one or more selectable marker genes, for example an ampicillin resistance gene in the case of a bacterial plasmid or a neomycin resistance gene for a mammalian vector. The expression of an agent of the present invention or target of the present invention may be constitutive such that they are continually produced, or inducible, requiring a stimulus to initiate expression. In the case of inducible expression, production of an agent of the present invention or a target can be initiated when required by, for example, addition of an inducer substance to the culture medium, for example dexamethasone or IPTG.

Fusion Proteins

The NPY or NPY Y1 or an agent (i.e. NPYI or NPY Y1i) of the present invention may be expressed as a fusion protein to aid extraction and purification and/or delivery of the agent of the present invention or the NPY/NPY Y1 receptor target to an individual and/or to facilitate the development of a screen for agents. Examples of fusion protein partners include glutathione-S-transferase (GST), 6× His, GAL4 (DNA binding and/or transcriptional activation domains) and β-galactosidase. It may also be convenient to include a proteolytic cleavage site between the fusion protein partner and the protein sequence of interest to allow removal of fusion protein sequences. Preferably the fusion protein will not hinder the activity of the target.

The fusion protein may comprise an antigen or an antigenic determinant fused to the substance of the present invention. In this embodiment, the fusion protein may be a non-naturally occurring fusion protein comprising a substance which may act as an adjuvant in the sense of providing a generalised stimulation of the immune system. The antigen or antigenic determinant may be attached to either the amino or carboxy terminus of the substance.

In another embodiment of the invention, the amino acid sequence may be ligated to a heterologous sequence to encode a fusion protein. For example, for screening of peptide libraries for agents capable of affecting the substance activity, it may be useful to encode a chimeric substance expressing a heterologous epitope that is recognised by a commercially available antibody.

Host Cells

A wide variety of host cells can be employed for expression of the nucleotide sequences encoding the agent—such as an agent of the present invention—or a NPY/NPY Y1 receptor target of the present invention. These cells may be both prokaryotic and eukaryotic host cells. Suitable host cells include bacteria such as E. coli, yeast, filamentous fungi, insect cells, mammalian cells, typically immortalized, e.g., mouse, CHO, human and monkey cell lines and derivatives thereof.

Examples of suitable expression hosts within the scope of the present invention are fungi such as Aspergillus species (such as those described in EP-A-01 84438 and EP-A-0284603) and Trichoderma species; bacteria such as Bacillus species (such as those described in EP-A-01 34048 and EP-A-0253455), Streptomyces species and Pseudomonas species; and yeasts such as Kluyveromyces species (such as those described in EP-A-0096430 and EP-A-0301670) and Saccharomyces species. By way of example, typical expression hosts may be selected from Aspergillus niger, Aspergillus niger var. tubigenis, Aspergillus niger var. awamori, Aspergillus aculeatis, Aspergillus nidulans, Aspergillus orvzae, Trichoderma reesei, Bacillus subtilis, Bacillus licheniformis, Bacillus amyloliquefaciens, Kluyveromyces lactis and Saccharomyces cerevisiae.

The use of suitable host cells—such as yeast, fungal and plant host cells—may provide for post-translational modifications (e.g. myristoylation, glycosylation, truncation, lapidation and tyrosine, serine or threonine phosphorylation) as may be needed to confer optimal biological activity on recombinant expression products of the present invention.

Preferred host cells are able to process the expression products to produce an appropriate mature polypeptide. Examples of processing includes but is not limited to glycosylation, ubiquitination, disulfide bond formation and general post-translational modification.

Antibodies

In one embodiment of the present invention, the agent may be an antibody. In addition, or in the alternative, the target may be an antibody.

Antibodies may be produced by standard techniques, such as by immunisation with the substance of the invention or by using a phage display library.

For the purposes of this invention, the term “antibody”, unless specified to the contrary, includes but is not limited to, polyclonal, monoclonal, chimeric, single chain, Fab fragments, fragments produced by a Fab expression library, as well as mimetics thereof. Such fragments include fragments of whole antibodies which retain their binding activity for a target substance, Fv, F(ab′) and F(ab′)₂ fragments, as well as single chain antibodies (scFv), fusion proteins and other synthetic proteins which comprise the antigen-binding site of the antibody. Furthermore, the antibodies and fragments thereof may be humanised antibodies. Neutralising antibodies, i.e., those which inhibit biological activity of the substance polypeptides, are especially preferred for diagnostics and therapeutics.

If polyclonal antibodies are desired, a selected mammal (e.g., mouse, rabbit, goat, horse, etc.) is immunised with an immunogenic polypeptide bearing a epitope(s) obtainable from an identified agent and/or substance of the present invention. Depending on the host species, various adjuvants may be used to increase immunological response. Such adjuvants include, but are not limited to, Freund's, mineral gels such as aluminium hydroxide, and surface active substances such as lysolecithin, pluronic polyols, polyanions, peptides, oil emulsions, keyhole limpet hemocyanin, and dinitrophenol. BCG (Bacilli Calmette-Guerin) and Corynebacterium parvum are potentially useful human adjuvants which may be employed if purified the substance polypeptide is administered to immunologically compromised individuals for the purpose of stimulating systemic defence.

Serum from the immunised animal is collected and treated according to known procedures. If serum containing polyclonal antibodies to an epitope obtainable from an identified agent and/or substance of the present invention contains antibodies to other antigens, the polyclonal antibodies can be purified by immunoaffinity chromatography. Techniques for producing and processing polyclonal antisera are known in the art. In order that such antibodies may be made, the invention also provides polypeptides of the invention or fragments thereof haptenised to another polypeptide for use as immunogens in animals or humans.

Monoclonal antibodies directed against epitopes obtainable from an identified agent and/or substance of the present invention can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced against orbit epitopes can be screened for various properties; i.e., for isotype and epitope affinity.

Monoclonal antibodies to the substance and/or identified agent may be prepared using any technique which provides for the production of antibody molecules by continuous cell lines in culture. These include, but are not limited to, the hybridoma technique originally described by Koehler and Milstein (1975 Nature 256:495-497), the human B-cell hybridoma technique (Kosbor et al (1983) Immunol Today 4:72; Cote et al (1983) Proc Natl Acad Sci 80:2026-2030) and the EBV-hybridoma technique (Cole et al (1985) Monoclonal Antibodies and Cancer Therapy, Alan R Liss Inc, pp 77-96). In addition, techniques developed for the production of “chimeric antibodies”, the splicing of mouse antibody genes to human antibody genes to obtain a molecule with appropriate antigen specificity and biological activity can be used (Morrison et al (1984) Proc Natl Acad Sci 81:6851-6855; Neuberger et al (1984) Nature 312:604-608; Takeda et al (1985) Nature 314:452-454). Alternatively, techniques described for the production of single chain antibodies (U.S. Pat. No. 4,946,779) can be adapted to produce the substance specific single chain antibodies.

Antibodies, both monoclonal and polyclonal, which are directed against epitopes obtainable from an identified agent and/or substance are particularly useful in diagnosis, and those which are neutralising are useful in passive immunotherapy. Monoclonal antibodies, in particular, may be used to raise anti-idiotype antibodies. Anti-idiotype antibodies are immunoglobulins which carry an “internal image” of the substance and/or agent against which protection is desired. Techniques for raising anti-idiotype antibodies are known in the art. These anti-idiotype antibodies may also be useful in therapy.

Antibodies may also be produced by inducing in vivo production in the lymphocyte population or by screening recombinant immunoglobulin libraries or panels of highly specific binding reagents as disclosed in Oriandi et al (1989, Proc Natl Acad Sci 86: 3833-3837), and Winter G and Milstein C (1991; Nature 349:293-299).

Antibody fragments which contain specific binding sites for the substance may also be generated. For example, such fragments include, but are not limited to, the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed to allow rapid and easy identification of monoclonal Fab fragments with the desired specificity (Huse WD et al (1989) Science 256:1275-1281).

Reporters

A wide variety of reporters may be used in the assay methods (as well as screens) of the present invention with preferred reporters providing conveniently detectable signals (e.g. by spectroscopy). By way of example, a reporter gene may encode an enzyme which catalyses a reaction which alters light absorption properties.

Examples of reporter molecules include but are not limited to β-galactosidase, invertase, green fluorescent protein, luciferase, chloramphenicol, acetyltransferase, β-glucuronidase, exo-glucanase and glucoamylase. Alternatively, radiolabelled or fluorescent tag-labelled nucleotides can be incorporated into nascent transcripts which are then identified when bound to oligonucleotide probes.

In one preferred embodiment, the production of the reporter molecule is measured by the enzymatic activity of the reporter gene product, such as β-galactosidase.

A variety of protocols for detecting and measuring the expression of the target, such as by using either polyclonal or monoclonal antibodies specific for the protein, are known in the art. Examples include enzyme-linked immunosorbent assay (ELISA), radioimmunoassay (RIA) and fluorescent activated cell sorting (FACS). A two-site, monoclonal-based immunoassay utilising monoclonal antibodies reactive to two non-interfering epitopes on polypeptides is preferred, but a competitive binding assay may be employed. These and other assays are described, among other places, in Hampton R et al (1990, Serological Methods, A Laboratory Manual, APS Press, St Paul Minn.) and Maddox DE et al (1983, J Exp Med 158:121 1).

A wide variety of labels and conjugation techniques are known by those skilled in the art and can be used in various nucleic and amino acid assays. Means for producing labelled hybridisation or PCR probes for detecting the target polynucleotide sequences include oligolabelling, nick translation, end-labelling or PCR amplification using a labelled nucleotide. Alternatively, the coding sequence, or any portion of it, may be cloned into a vector for the production of an mRNA probe. Such vectors are known in the art, are commercially available, and may be used to synthesise RNA probes in vitro by addition of an appropriate RNA polymerase such as T7, T3 or SP6 and labelled nucleotides.

A number of companies such as Pharmacia Biotech (Piscataway, N.J.), Promega (Madison, Wis.), and US Biochemical Corp (Cleveland, Ohio) supply commercial kits and protocols for these procedures. Suitable reporter molecules or labels include those radionuclides, enzymes, fluorescent, chemiluminescent, or chromogenic agents as well as substrates, cofactors, inhibitors, magnetic particles and the like. Patents teaching the use of such labels include U.S. Pat. Nos. 3,817,837; 3,850,752; 3,939,350; 3,996,345; 4,277,437; 4,275,149 and 4,366,241. Also, recombinant immunoglobulins may be produced as shown in U.S. Pat. No. 4,816,567.

Additional methods to quantify the expression of a particular molecule include radiolabeling (Melby P C et al 1993 J Immunol Methods 159:235-44) or biotinylating (Duplaa C et al 1993 Anal Biochem 229-36) nucleotides, coamplification of a control nucleic acid, and standard curves onto which the experimental results are interpolated. Quantification of multiple samples may be speeded up by running the assay in an ELISA format where the oligomer of interest is presented in various dilutions and a spectrophotometric or calorimetric response gives rapid quantification.

Although the presence/absence of marker gene expression suggests that the gene of interest is also present, its presence and expression should be confirmed. For example, if the nucleotide sequence is inserted within a marker gene sequence, recombinant cells containing the same may be identified by the absence of marker gene function. Alternatively, a marker gene can be placed in tandem with a target coding sequence under the control of a single promoter. Expression of the marker gene in response to induction or selection usually indicates expression of the target as well.

Alternatively, host cells which contain the coding sequence for the target and express the target coding regions may be identified by a variety of procedures known to those of skill in the art. These procedures include, but are not limited to, DNA-DNA or DNA-RNA hybridisation and protein bioassay or immunoassay techniques which include membrane-based, solution-based, or chip-based technologies for the detection and/or quantification of the nucleic acid or protein.

Screens

Any one or more of appropriate targets—such as an amino acid sequence and/or nucleotide sequence—may be used for identifying an agent, e.g. NPYi or an NPY Y1i, in any of a variety of drug screening techniques. The target employed in such a test may be free in solution, affixed to a solid support, borne on a cell surface, or located intracellularly. The target may even be within an animal model, wherein said target may be an exogenous target or an introduced target. The animal model will be a non-human animal model. The abolition of target activity or the formation of binding complexes between the target and the agent being tested may be measured.

Techniques for drug screening may be based on the method described in Geysen, European Patent Application 84/03564, published on Sep. 13, 1984. In summary, large numbers of different small peptide test compounds are synthesised on a solid substrate, such as plastic pins or some other surface. The peptide test compounds are reacted with a suitable target or fragment thereof and washed. Bound entities are then detected—such as by appropriately adapting methods well known in the art. A purified target can also be coated directly onto plates for use in a drug screening techniques. Alternatively, non-neutralising antibodies can be used to capture the peptide and immobilise it on a solid support.

This invention also contemplates the use of competitive drug screening assays in which neutralising antibodies capable of binding a target specifically compete with a test compound for binding to a target.

Another technique for screening provides for high throughput screening (HTS) of agents having suitable binding affinity to the substances and is based upon the method described in detail in WO 84/03564.

It is expected that the assay methods of the present invention will be suitable for both small and large-scale screening of test compounds as well as in quantitative assays.

In a preferred aspect, the screen of the present invention comprises at least the following steps (which need not be in this same consecutive order): (a) conducting an in vitro screen to determine whether a candidate agent has the relevant activity (such as modulation of NPY, in particular NPY Y1, such as NPY or NPY Y1 from dog kidney); (b) conducting one or more selectivity screens to determine the selectivity of said candidate agent (e.g. to see if said agent is also an ACE inhibitor—such as by using the assay protocol presented herein and/or see if said agent is active against NPY Y2 and/or NPY Y5); and (c) conducting an in vivo screen with said candidate agent (e.g. using a functional animal model, including determining the selectivity of the agent by determining the effect of the agent on arterial blood pressure). Typically, if said candidate agent passes screen (a) and screen (b) then screen (c) is performed.

Diagnostics

The present invention also provides a diagnostic composition or kit for the detection of a pre-disposition for MED. In this respect, the composition or kit will comprise an entity that is capable of indicating the presence of one or more—or even the absence of one or more—of the targets in a test sample. Preferably, the test sample is obtained from the penis.

By way of example, the diagnostic composition may comprise any one of the nucleotide sequences mentioned herein or a variant, homologue, fragment or derivative thereof, or a sequence capable of hybridising to all or part of any one of the nucleotide sequence.

In order to provide a basis for the diagnosis of disease, normal or standard values from a target should be established. This may be accomplished by combining body fluids or cell extracts taken from normal subjects, either animal or human, with an antibody to a target under conditions suitable for complex formation which are well known in the art. The amount of standard complex formation may be quantified by comparing it to a dilution series of positive controls where a known amount of antibody is combined with known concentrations of a purified target. Then, standard values obtained from normal samples may be compared with values obtained from samples from subjects potentially affected by MED. Deviation between standard and subject values establishes the presence of the disease state.

A target itself, or any part thereof, may provide the basis for a diagnostic and/or a therapeutic compound. For diagnostic purposes, target polynucleotide sequences may be used to detect and quantify gene expression in conditions, disorders or diseases in which MED may be implicated.

The target encoding polynucleotide sequence may be used for the diagnosis of MED resulting from expression of the target. For example, polynucleotide sequences encoding a target may be used in hybridisation or PCR assays of tissues from biopsies or autopsies or biological fluids, to detect abnormalities in target expression. The form of such qualitative or quantitative methods may include Southern or northern analysis, dot blot or other membrane-based technologies; PCR technologies; dip stick, pin or chip technologies; and ELISA or other multiple sample formal technologies. All of these techniques are well known in the art and are in fact the basis of many commercially available diagnostic kits.

Such assays may be tailored to evaluate the efficacy of a particular therapeutic treatment regime and may be used in animal studies, in clinical trials, or in monitoring the treatment of an individual. In order to provide a basis for the diagnosis of disease, a normal or standard profile for target expression should be established. This is accomplished by combining body fluids or cell extracts taken from normal subjects, either animal or human, with the target or a portion thereof, under conditions suitable for hybridisation or amplification. Standard hybridisation may be quantified by comparing the values obtained for normal subjects with a dilution series of positive controls run in the same experiment where a known amount of purified target is used. Standard values obtained from normal samples may be compared with values obtained from samples from subjects potentially affected by a disorder or disease related to expression of the target coding sequence. Deviation between standard and subject values establishes the presence of the disease state. If disease is established, an existing therapeutic agent is administered, and treatment profile or values may be generated. Finally, the assay may be repeated on a regular basis to evaluate whether the values progress toward or return to the normal or standard pattern. Successive treatment profiles may be used to show the efficacy of treatment over a period of several days or several months.

Thus, in one aspect, the present invention relates to the use of a target polypeptide, or variant, homologue, fragment or derivative thereof, to produce anti-target antibodies which can, for example, be used diagnostically to detect and quantify target levels in MED.

The present invention further provides diagnostic assays and kits for the detection of a target in cells and tissues comprising a purified target which may be used as a positive control, and anti-target antibodies. Such antibodies may be used in solution-based, membrane-based, or tissue-based technologies to detect any disease state or condition related to the expression of target protein or expression of deletions or a variant, homologue, fragment or derivative thereof.

Diagnostic Kits

The present invention also includes a diagnostic composition or diagnostic methods or kits for (i) detection and measurement of NPY and NPY Y1 activity in biological fluids and tissue; and/or (ii) localisation of a NPY and NPY Y1 activity in erectile tissues; and/or for (iii) the detection of a predisposition to a male sexual dysfunction, such as MED. In this respect, the composition or kit will comprise an entity that is capable of indicating the presence of one or more—or even the absence of one or more—targets, such as NPY or NPY Y1 activity in a test sample. Preferably, the test sample is obtained from male sexual genitalia or a secretion thereof or therefrom.

By way of example, the diagnostic composition may comprise any one of the nucleotide sequences mentioned herein or a variant, homologue, fragment or derivative thereof, or a sequence capable of hybridising to all or part of any one of the nucleotide sequence.

Diagnostic Testing

In order to provide a basis for the diagnosis of disease, normal or standard values from a target should be established. This may be accomplished by combining body fluids or cell extracts taken from normal subjects, either animal or human, with, for example, an antibody to a target under conditions suitable for complex formation which are well known in the art. The amount of standard complex formation may be quantified by comparing it to a dilution series of positive controls where a known amount of antibody is combined with known concentrations of a purified target. Then, standard values obtained from normal samples may be compared with values obtained from samples from subjects potentially affected by a male sexual dysfunction (such as MED). Deviation between standard and subject values establishes the presence of the disease state.

A target itself, or any part thereof, may provide the basis for a diagnostic and/or a prophylactic and/or therapeutic compound. For diagnostic purposes, target polynucleotide sequences may be used to detect and quantify gene expression in conditions, disorders or diseases in which male sexual dysfunction, particularly MED, may be implicated.

The target encoding polynucleotide sequence may be used for the diagnosis of SD resulting from expression of the target. For example, polynucleotide sequences encoding a target may be used in hybridisation or PCR assays of tissues from biopsies or autopsies or biological fluids, to detect abnormalities in target expression. The form of such qualitative or quantitative methods may include Southern or northern analysis, dot blot or other membrane-based technologies; PCR technologies; dip stick, pin or chip technologies; and ELISA or other multiple sample formal technologies. All of these techniques are well known in the art and are in fact the basis of many commercially available diagnostic kits.

Such assays may be tailored to evaluate the efficacy of a particular therapeutic treatment regime and may be used in animal studies, in clinical trials, or in monitoring the treatment of an individual. In order to provide a basis for the diagnosis of disease, a normal or standard profile for target expression should be established. This is accomplished by combining body fluids or cell extracts taken from normal subjects, either animal or human, with the target or a portion thereof, under conditions suitable for hybridisation or amplification. Standard hybridisation may be quantified by comparing the values obtained for normal subjects with a dilution series of positive controls run in the same experiment where a known amount of purified target is used. Standard values obtained from normal samples may be compared with values obtained from samples from subjects potentially affected by a disorder or disease related to expression of the target coding sequence. Deviation between standard and subject values establishes the presence of the disease state. If disease is established, an existing therapeutic agent is administered, and treatment profile or values may be generated. Finally, the assay may be repeated on a regular basis to evaluate whether the values progress toward or return to the normal or standard pattern. Successive treatment profiles may be used to show the efficacy of treatment over a period of several days or several months.

Thus, in one aspect, the present invention relates to the use of a target polypeptide, or variant, homologue, fragment or derivative thereof, to produce anti-target antibodies which can, for example, be used diagnostically to detect and quantify target levels in male sexual dysfunctioning states.

The present invention further provides diagnostic assays and kits for the detection of a target in cells and tissues comprising a purified target which may be used as a positive control, and anti-target antibodies. Such antibodies may be used in solution-based, membrane-based, or tissue-based technologies to detect any disease state or condition related to the expression of target protein or expression of deletions or a variant, homologue, fragment or derivative thereof.

The diagnostic compositions and/or kits comprising these entities may be used for a rapid, reliable, sensitive, and specific measurement and localisation of NPY or NPY Y1 activity in erectile tissue extracts. In certain situations, the kit may indicate the existence of male sexual dysfunction, such as MED.

Assay Methods

The diagnostic compositions and/or methods and/or kits may be used in the following techniques which include but are not limited to; competitive and non-competitive assays, radioimmunoassay, bioluminescence and chemiluminescence assays, fluorometric assays, sandwich assays, immunoradiometric assays, dot blots, enzyme linked assays including ELISA, microtiter plates, antibody coated strips or dipsticks for rapid monitoring of urine or blood, immunohistochemistry and immunocytochemistry.

By way of example, an immunohistochemistry kit may also be used for localisation of NPY or NPY Y1 activity in genital tissue. This immunohistochemistry kit permits localisation of NPY or NPY Y1in tissue sections and cultured cells using both light and electron microscopy which may be used for both research and clinical purposes. Such information may be useful for diagnostic and possibly therapeutic purposes in the detection and/or prevention and/or treatment of MED. For each kit the range, sensitivity, precision, reliability, specificity and reproducibility of the assay are established. Intraassay and interassay variation is established at 20%, 50% and 80% points on the standard curves of displacement or activity.

PROBES

Another aspect of the subject invention is the provision of nucleic acid hybridisation or PCR probes which are capable of detecting (especially those that are capable of selectively selecting) polynucleotide sequences, including genomic sequences, encoding a target coding region, such as an NPY or an NPY Y1 receptor, or closely related molecules, such as alleles. The specificity of the probe, i.e., whether it is derived from a highly conserved, conserved or non-conserved region or domain, and the stringency of the hybridisation or amplification (high, intermediate or low) will determine whether the probe identifies only naturally occurring target coding sequence, or related sequences. Probes for the detection of related nucleic acid sequences are selected from conserved or highly conserved nucleotide regions of target family members and such probes may be used in a pool of degenerate probes. For the detection of identical nucleic acid sequences, or where maximum specificity is desired, nucleic acid probes are selected from the non-conserved nucleotide regions or unique regions of the target polynucleotides. As used herein, the term “non-conserved nucleotide region” refers to a nucleotide region that is unique to a target coding sequence disclosed herein and does not occur in related family members.

PCR as described in U.S. Pat. Nos. 4,683,195, 4,800,195 and 4,965,188 provides additional uses for oligonucleotides based upon target sequences. Such oligomers are generally chemically synthesised, but they may be generated enzymatically or produced from a recombinant source. Oligomers generally comprise two nucleotide sequences, one with sense orientation (5′→3′) and one with antisense (3′→5′) employed under optimised conditions for identification of a specific gene or condition. The same two oligomers, nested sets of oligomers, or even a degenerate pool of oligomers may be employed under less stringent conditions for detection and/or quantification of closely related DNA or RNA sequences.

The nucleic acid sequence for an agent or a target can also be used to generate hybridisation probes as previously described, for mapping the endogenous genomic sequence. The sequence may be mapped to a particular chromosome or to a specific region of the chromosome using well known techniques. These include in situ hybridisation to chromosomal spreads (Verma et al (1988) Human Chromosomes: A Manual of Basic Techniques, Pergamon Press, New York City), flow-sorted chromosomal preparations, or artificial chromosome constructions such as YACs, bacterial artificial chromosomes (BACs), bacterial Pi constructions or single chromosome cDNA libraries.

In situ hybridisation of chromosomal preparations and physical mapping techniques such as linkage analysis using established chromosomal markers are invaluable in extending genetic maps. Examples of genetic maps can be found in Science (1995; 270:410f and 1994; 265:1981f). Often the placement of a gene on the chromosome of another mammalian species may reveal associated markers even if the number or arm of a particular human chromosome is not known. New sequences can be assigned to chromosomal arms, or parts thereof, by physical mapping. This provides valuable information to investigators searching for disease genes using positional cloning or other gene discovery techniques. Once a disease or syndrome has been crudely localised by genetic linkage to a particular genomic region any sequences mapping to that area may represent associated or regulatory genes for further investigation. The nucleotide sequence of the subject invention may also be used to detect differences in the chromosomal location due to translocation, inversion, etc. between normal, carrier or affected individuals.

Organism

The term “organism” in relation to the present invention includes any organism that could comprise the target and/or products obtained therefrom. Examples of organisms may include a mammal, a fungus, yeast or a plant.

The term “transgenic organism” in relation to the present invention includes any organism that comprises the target and/or products obtained therefrom.

Transformation of Host Cells/Host Organisms

As indicated earlier, the host organism can be a prokaryotic or a eukaryotic organism. Examples of suitable prokaryotic hosts include E coli and Bacillus subtilis. Teachings on the transformation of prokaryotic hosts is well documented in the art, for example see Sambrook et al (Molecular Cloning: A Laboratory Manual, 2nd edition, 1989, Cold Spring Harbor Laboratory Press) and Ausubel et al., Current Protocols in Molecular Biology (1995), John Wiley & Sons, Inc.

If a prokaryotic host is used then the nucleotide sequence may need to be suitably modified before transformation—such as by removal of introns.

In another embodiment the transgenic organism can be a yeast. In this regard, yeast have also been widely used as a vehicle for heterologous gene expression. The species Saccharomyces cerevisiae has a long history of industrial use, including its use for heterologous gene expression. Expression of heterologous genes in Saccharomyces cerevisiae has been reviewed by Goodey et al (1987, Yeast Biotechnology, D R Berry et al, eds, pp 401-429, Allen and Unwin, London) and by King et al (1989, Molecular and Cell Biology of Yeasts, E F Walton and G T Yarronton, eds, pp 107-133, Blackie, Glasgow).

For several reasons Saccharomyces cerevisiae is well suited for heterologous gene expression. First, it is non-pathogenic to humans and it is incapable of producing certain endotoxins. Second, it has a long history of safe use following centuries of commercial exploitation for various purposes. This has led to wide public acceptability. Third, the extensive commercial use and research devoted to the organism has resulted in a wealth of knowledge about the genetics and physiology as well as large-scale fermentation characteristics of Saccharomyces cerevisiae.

A review of the principles of heterologous gene expression in Saccharomyces cerevisiae and secretion of gene products is given by E Hinchcliffe E Kenny (1993, “Yeast as a vehicle for the expression of heterologous genes”, Yeasts, Vol. 5, Anthony H Rose and J Stuart Harrison, eds, 2nd edition, Academic Press Ltd.).

Several types of yeast vectors are available, including integrative vectors, which require recombination with the host genome for their maintenance, and autonomously replicating plasmid vectors.

In order to prepare the transgenic Saccharomyces, expression constructs are prepared by inserting the nucleotide sequence of the present invention into a construct designed for expression in yeast. Several types of constructs used for heterologous expression have been developed. The constructs contain a promoter active in yeast fused to the nucleotide sequence of the present invention, usually a promoter of yeast origin, such as the GAL1 promoter, is used. Usually a signal sequence of yeast origin, such as the sequence encoding the SUC2 signal peptide, is used. A terminator active in yeast ends the expression system.

For the transformation of yeast several transformation protocols have been developed. For example, a transgenic Saccharomyces according to the present invention can be prepared by following the teachings of Hinnen et al (1978, Proceedings of the National Academy of Sciences of the USA 75, 1929); Beggs, J D (1978, Nature, London, 275, 104); and Ito, H et al (1983, J Bacteriology 153, 163-168).

The transformed yeast cells are selected using various selective markers. Among the markers used for transformation are a number of auxotrophic markers such as LEU2, HIS4 and TRP1, and dominant antibiotic resistance markers such as aminoglycoside antibiotic markers, e.g. G418.

Another host organism is a plant. The basic principle in the construction of genetically modified plants is to insert genetic information in the plant genome so as to obtain a stable maintenance of the inserted genetic material. Several techniques exist for inserting the genetic information, the two main principles being direct introduction of the genetic information and introduction of the genetic information by use of a vector system. A review of the general techniques may be found in articles by Potrykus (Annu Rev Plant Physiol Plant Mol Biol [1991] 42:205-225) and Christou (Agro-Food-Industry Hi-Tech March/April 17-27, 1994). Further teachings on plant transformation may be found in EP-A-0449375.

Thus, the present invention also provides a method of transforming a host cell with a nucleotide sequence that is to be the target or is to express the target. Host cells transformed with the nucleotide sequence may be cultured under conditions suitable for the expression and recovery of the encoded protein from cell culture. The protein produced by a recombinant cell may be secreted or may be contained intracellularly depending on the sequence and/or the vector used. As will be understood by those of skill in the art, expression vectors containing coding sequences can be designed with signal sequences which direct secretion of the coding sequences through a particular prokaryotic or eukaryotic cell membrane. Other recombinant constructions may join the coding sequence to nucleotide sequence encoding a polypeptide domain which will facilitate purification of soluble proteins (Kroll D J et al (1993) DNA Cell Biol 12:441-53).

ACE Assay

Potency values for ACE or selectivity values for inhibitors of NPY or NPY Y1 over ACE are determined by the following assay.

The Preparation and Assay of Soluble Angiotensin Converting Enzyme (Ace), from Porcine and Human Kidney Cortex.

Soluble ACE activity is obtained from the kidney cortex and assayed by measuring the rate of cleavage of the ACE substrate Abz-Gly-p-nitro-Phe-Pro-OH to generate its fluorescent product, Abz-Gly.

1. Materials

All water is double de ionised.

1.1 Human Kidney IIAM (Pennsylvania. U.S.A.) or UK Human Tissue Bank (UK HTB)

1.2 Porcine kidney ACE Sigma (A2580)

1.3 Homogenisation buffer-1

100 mM Mannitol and 20 mM Tris@pH 7.1

2.42 g Tris (Fisher T/P630/60) is diluted in 1 liter of water and the pH adjusted to 7.1 using 6M HCl at room temperature. To this 18.22 g Mannitol (Sigma M-9546) is added.

1.4 Homogenisation buffer-2

100 mM Mannitol, 20 mM Tris@pH 7.1 and 10 mM MgCl_{2.}6H₂O (Fisher M0600/53)

To 500 ml of the homogenisation buffer 1 (1.4) 1.017 g of MgCl₂ is added.

1.5 Tris buffer (ACE buffer).

50 mM Tris and 300 mM NaCl@pH 7.4

50 ml of 50 mM Tris pH 7.4 (Sigma T2663) and 17.52 g NaCl (Fisher S/3160/60) are made up to 1000 ml in water.

1.6 Substrate (Abz-D-Gly-p-nitro-Phe-Pro-OH) (Bachem M-1100)

ACE substrate is stored as a powder at −20° C. A 2 mM stock is made by gently re-suspending the substrate in ACE buffer, this must not be vortexed or sonicated. 400 μl aliquots of the 2 mM stock are stored at −20° C. for up to one month.

1.7 Total product

Samples corresponding to 100% substrate to product conversion are included on the plate to enable the % substrate turnover to be determined (see calculations). The total product is generated by incubating 1 ml of 2 mM substrate with 20 μl of enzyme stock for 24 hours at 37° C.

1.8 Stop solution.

0.5M EDTA (Promega CAS[6081/92/6]) is diluted 1:250 in ACE buffer to make a 2 mM solution.

1.9 Dimethyl sulphoxide (DMSO).

1.10 Magnesium Chloride —MgCl₂.6H₂O (Fisher M0600/53).

1.11 Black 96 well flat bottom assay plates (Costar 3915 or Packard).

1.12 Topseal A (Packard 6005185).

1.13 Centrifuge tubes

2. Specific Equiptment

2.1 Sorvall RC-5B centrifuge (SS34 GSA rotor, pre-cooled to 4° C.).

2.2 Braun miniprimer mixer.

2.3 Beckman CS-6R centrifuge.

2.4 BMG Fluostar Galaxy.

2.5 Wesbart 1589 shaking incubator.

3. Methods

3.1 Tissue Preparation

3.1 Human ACE is obtained from the kidney cortex using a method adapted from Booth, A. G. & Kenny, A. J. (1974) Biochem. J. 142, 575-581.

3.3 Frozen kidneys are allowed to thaw at room temperature and the cortex is dissected away from the medulla.

3.4 The cortex is finely chopped and homogenised in approximately 10 volumes of homogenisation buffer-1 (1.4) using a Braun miniprimer (2.2).

3.5 Magnesium chloride (1.11) (20.3 mg/gm tissue) is added to the homogenate and stirred in an ice-water bath for 15 minutes.

3.6 The homogenate is centrifuged at 1,500 g (3,820 rpm) for 12 minutes in a Beckman centrifuge (2.3) before removing the supernatant to a fresh centrifuge tube and discarding the pellet.

3.7 The supernatant is centrifuged at 15,000 g (12,100 rpm) for 12 minutes in a Sovall centrifuge (2.1) and the supernatant is discarded.

3.8 The pale pink layer on the top of the remaining pellet is removed and re-suspended in homogenisation buffer-2 (1.5) (5 ml buffer per 1 g tissue).

3.9 The suspension is centrifuged at 2,200 g (4,630 rpm) for 12 minutes in a Beckman centrifuge before discarding the pellet.

3.10 The supernatant is centrifuged at 15,000 g (12,100 rpm) for 12 minutes using the Sorvall centrifuge and the supernatant is discarded.

3.11 The final pellet is resuspended in homogenisation buffer-2 (0.5 ml buffer per 1 g tissue). A homogenous suspension is obtained using a Braun miniprimer. This is then frozen down in 100 μl aliquots to be assayed for NPY or NPY Y1 activity.

4.0 Determination of Ace Activity

The activity of the previously aliquoted ACE is measured by its ability to cleave the ACE specific peptide substrate.

Porcine ACE (1.2) is defrosted and resuspended in ACE buffer (1.6) at 0.004U/μl, this is frozen down in 50 μl aliquots.

4.1 A 4% DMSO/ACE buffer solution is made (4 mls DMSO in 96 mls ACE buffer).

4.2 Substrate (1.7), total product (1.8) and enzyme (1.1, 1.2, 1.3), are left on ice to thaw.

4.3 50 μl of 4% DMSO/ACE buffer solution is added to each well.

4.4 The 2 mM substrate stock is diluted 1:100 to make a 20 μM solution. 100 μl of 20 μM substrate is added to each well (final concentration in the assay 10 μM).

4.5 50 μl of a range of enzyme dilutions is added to initiate the reaction (usually 1:100, 1:200, 1:400, 1:800, 1:1600, and 1:3200 are used). 50 μl of ACE buffer is added to blank wells.

4.6 The 2 mM total product is diluted 1:200 to make 10 μM solution. 200 μl 10 μM product is added to the first four wells of a new plate.

4.7 Plates are incubated at 37° C. in a shaking incubator for 60 minutes.

4.8 The enzyme reaction is stopped by the addition of 100 μl 2 mM EDTA in ACE buffer and incubated at 37° C. in a shaking incubator for 20 minutes before being read on the BMG Fluostar Galaxy (ex320/em420).

5. Ace Inhibition Assays

5.1 Substrate, total product, and enzyme stocks are left on ice to thaw.

5.2 Compound stocks are made up in 100% DMSO and diluted 1:25 in ACE buffer to give a 4% DMSO solution. All further dilutions are carried out in a 4% DMSO/ACE buffer solution (4 mls DMSO in 96mls ACE buffer).

5.3 50 μl of compound, in duplicate, is added to the 96 well plate and 50 μl of 4% DMSO/ACE buffer is added to control and blank wells.

5.4 Steps 5.2 and 5.3 can be carried out either by hand or using the Packard multiprobe robots

5.5 The 2 mM substrate stock is diluted 1:100 in ACE buffer to make a 20 μM solution (10 μM final concentration in the assay) (110 μl of 2 mM substrate added to 10.89 ml buffer is enough for 1 plate).

5.6 The enzyme stock is diluted in ACE buffer, as determined from activity checks (4.0).

5.7 The 2 mM total product stock is diluted 1:200 in ACE buffer to make a 10 μM solution. 200 μl is added to the first four wells of a separate plate.

5.8 The 0.5 mM EDTA stock is diluted 1:250 to make a 2 mM stock (44 μl EDTA to 10.96 ml ACE buffer).

5.9 To each well of the 96 well plate the following reagents are added:

Table 1: Reagents added to 96 well plate.

| | Compound/ | Tris | | ACE | Total |
|---|---|---|---|---|---|
| | DMSO | Buffer | Substrate | enzyme | product |
| Samples | 2 μl compound | 50 μl | 100 μl | 50 μl | None |
| Controls | 2 μl DMSO | 50 μl | 100 μl | 50 μl | None |
| Blanks | 2 μl DMSO | 100 μl | 100 μl | None | None |
| Totals | 2 μl DMSO | None | None | None | 200 μl |

5.10 50 μl of the highest concentration of each compound used in the assay is added in duplicate to the same 96 well plate as the totals (5.7). 150 μl of ACE buffer is added to determine any compound fluorescence.

5.11 The reaction is initiated by the addition of the ACE enzyme before incubating at 37° C. for 1 hour in a shaking incubator.

5.12 The reaction is stopped by the addition of 100 μl 2 mM EDTA and incubated at 37° C. for 20 minutes in a shaking incubator, before being read on the BMG Fluostar Galaxy (ex320/em420).

6. Calculations

The activity of the ACE enzyme is determined in the presence and absence of compound and expressed as a percentage.

FU=Fluorescence units

(i) % Control activity (turnover of enzyme):

$$\frac{Mean FU of controls - Mean FU of blanks}{Mean FU of totals - Mean FU of blanks} \times 100$$

(ii) % Activity with inhibitor:

$$\frac{Mean FU of compound - Mean FU of blanks}{Mean FU of totals - Mean FU of blanks} \times 100$$

(iii) Activity expressed as % of control:

% Activity with inhibitor×100

% Control activity

OR

$$\frac{Mean FU of compound - Mean FU of blanks}{Mean FU of contr ols - Mean FU of blanks} \times 100$$

(iv) % Inhibition=100−% control

(v) For fluorescent compounds the mean FU of blanks containing compound (5.10) is deducted from the mean FU of compound values used to calculate the % Activity.

A sigmoidal dose-response curve is fitted to the % activities (% of control) vs compound concentration and IC₅₀ values calculated using LabStats fit-curve in Excel.

PDE action potency values referred to herein are determined by the following assays:

PDE5 inhibitor—Test Methods

Phosphodiesterase (PDE) Inhibitory Activity

Preferred PDE compounds suitable for use in accordance with the present invention are potent and selective cGMP PDE5 inhibitors. In vitro PDE inhibitory activities against cyclic guanosine 3′,5′-monophosphate (cGMP) and cyclic adenosine 3′,5′-monophosphate (cAMP) phosphodiesterases can be determined by measurement of their IC₅₀ values (the concentration of compound required for 50% inhibition of enzyme activity).

The required PDE enzymes can be isolated from a variety of sources, including human corpus cavernosum, human and rabbit platelets, human cardiac ventricle, human skeletal muscle and bovine retina, essentially by the method of W. J. Thompson and M. M. Appleman (Biochem., 1971, 10, 311). In particular, the cGMP-specific PDE (PDE5) and the cGMP-inhibited cAMP PDE (PDE3) can be obtained from human corpus cavernosum tissue, human platelets or rabbit platelets; the cGMP-stimulated PDE (PDE2) was obtained from human corpus cavernosum; the calcium/calmodulin (Ca/CAM)-dependent PDE (PDE1) from human cardiac ventricle; the cAMP-specific PDE (PDE4) from human skeletal muscle; and the photoreceptor PDE (PDE6) from bovine retina. Phosphodiesterases 7-11 can be generated from full length human recombinant clones transfected into SF9 cells.

Assays can be performed either using a modification of the “batch” method of W. J. Thompson et al. (Biochem., 1979, 18, 5228) or using a scintillation proximity assay for the direct detection of AMP/GMP using a modification of the protocol described by Amersham plc under product code TRKQ7090/7100. In summary, the effect of PDE inhibitors was investigated by assaying a fixed amount of enzyme in the presence of varying inhibitor concentrations and low substrate, (cGMP or cAMP in a 3:1 ratio unlabelled to [³H]-labeled at a conc ˜⅓ Kₘ) such that IC₅₀≅Kᵢ. The final assay volume was made up to 100 μl with assay buffer [20 mM Tris-HCl pH 7.4, 5 mM MgCl₂, 1 mg/ml bovine serum albumin]. Reactions were initiated with enzyme, incubated for 30-60 min at 30° C. to give <30% substrate turnover and terminated with 50 μl yttrium silicate SPA beads (containing 3 mM of the respective unlabelied cyclic nucleotide for PDEs 9 and 11). Plates were re-sealed and shaken for 20 min, after which the beads were allowed to settle for 30 min in the dark and then counted on a TopCount plate reader (Packard, Meriden, Conn.) Radioactivity units were converted to % activity of an uninhibited control (100%), plotted against inhibitor concentration and inhibitor IC₅₀ values obtained using the ‘Fit Curve’ Microsoft Excel extension.

Functional Activity

This can be assessed in vitro by determining the capacity of a compound of the invention to enhance sodium nitroprusside-induced relaxation of pre-contracted rabbit corpus cavernosum tissue strips, as described by S. A. Ballard et al. (Brit. J. Pharmacol., 1996, 118 (suppl.), abstract 153P).

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

By cross reference herein to compounds contained in patents which can be used in accordance with invention, we mean the therapeutically active compound as defined in the claims (in particular of claim 1) and the specific examples (all of which is incorporated herein by reference).

The invention will now be further described only by way of example in which reference is made to the following Figures:

### FIGS.

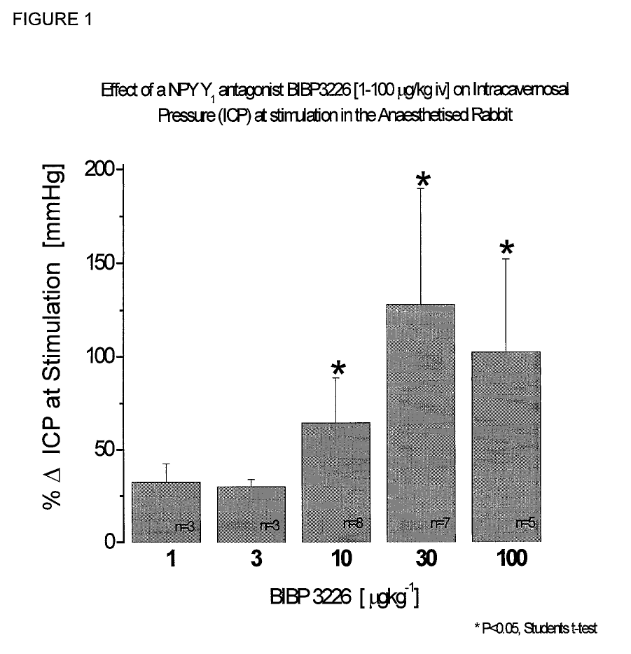

FIG. 1 which shows a graph;

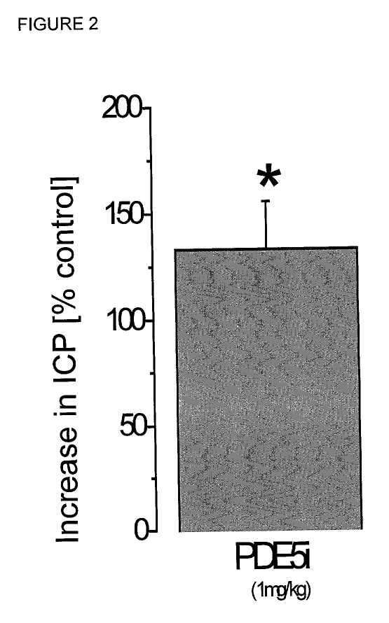

FIG. 2 which shows a graph;

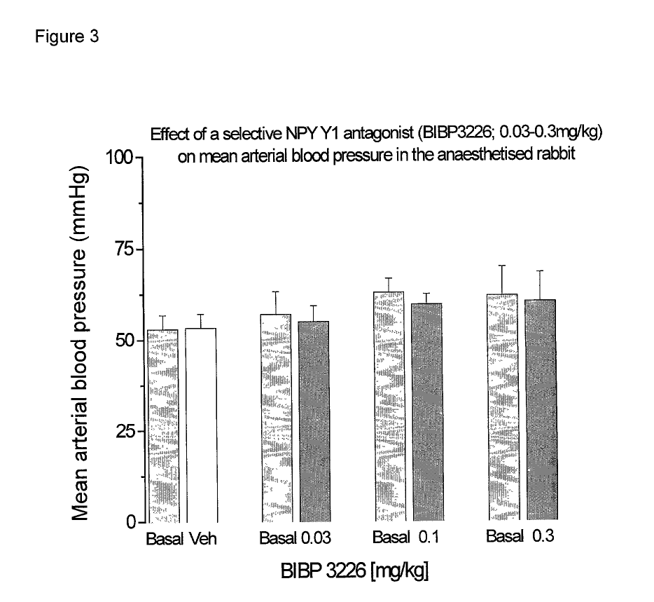

FIG. 3 which shows a graph;

FIG. 4 which shows a nucleotide sequence;

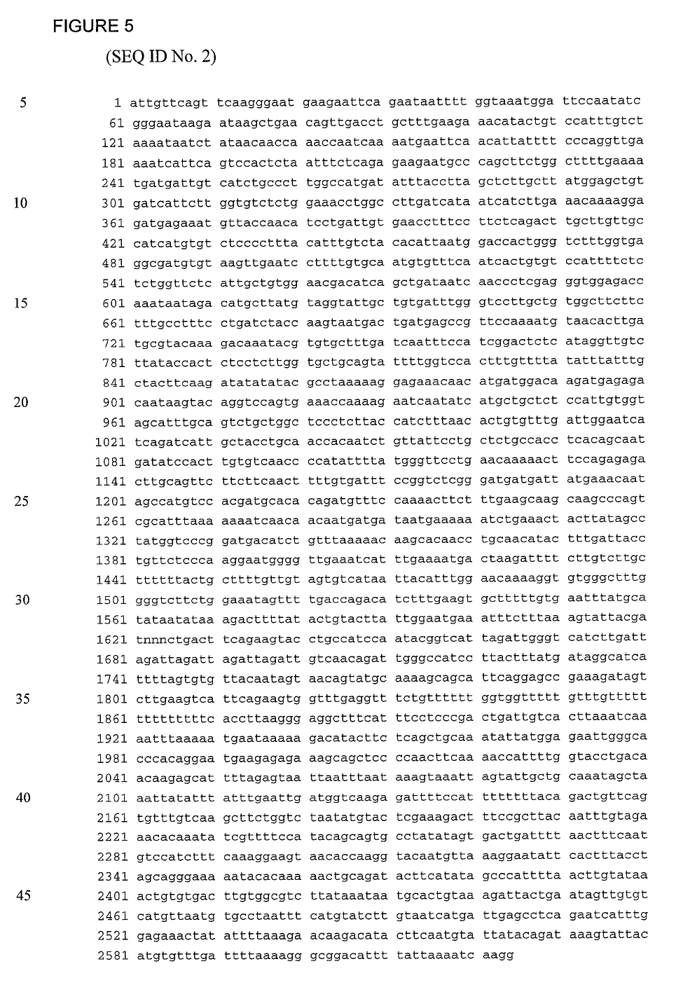

FIG. 5 which shows a nucleotide sequence;

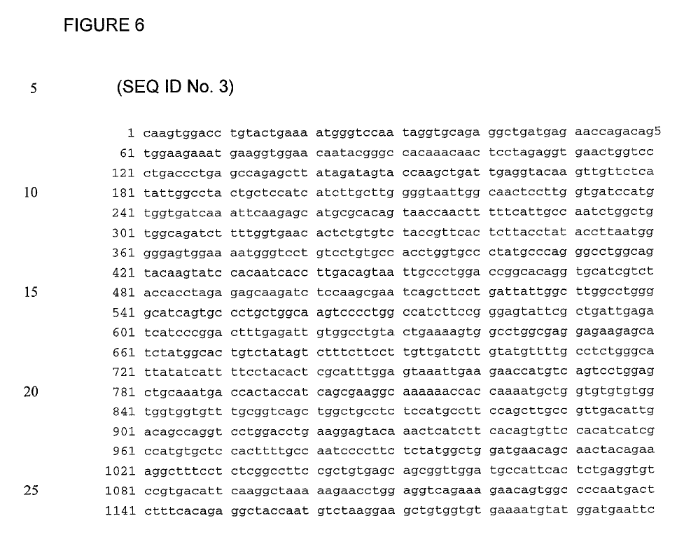

FIG. 6 which shows a nucleotide sequence;

FIG. 7 which shows a nucleotide sequence;

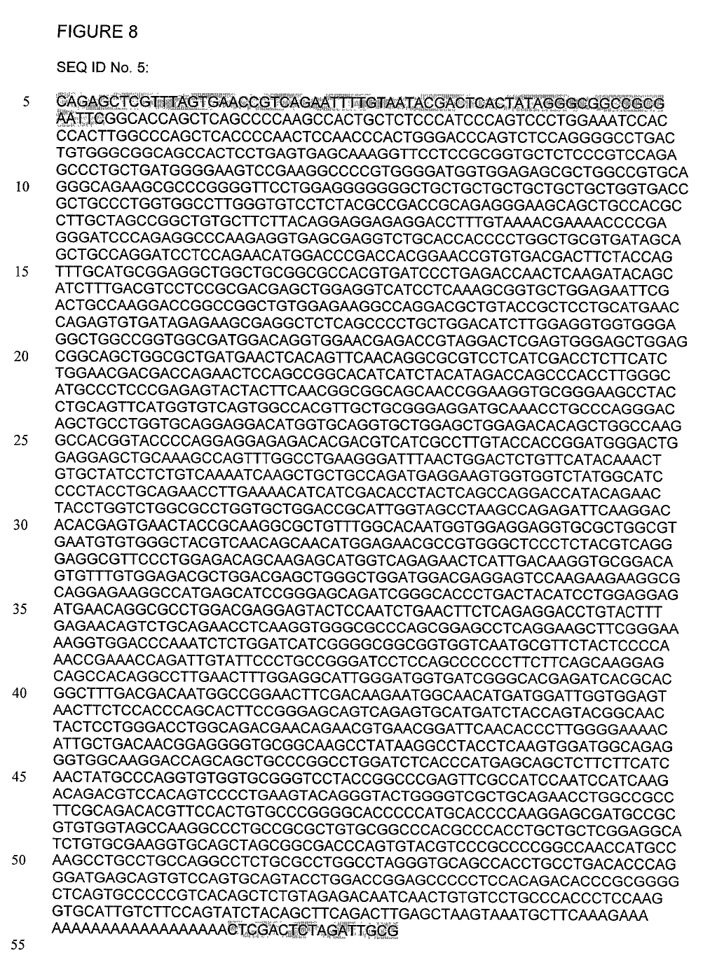

FIG. 8 which shows a nucleotide sequence;

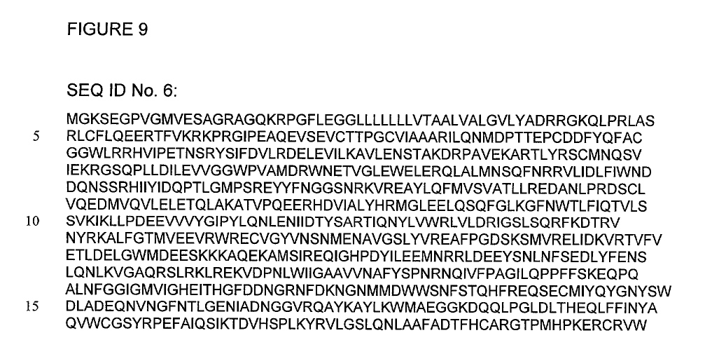

FIG. 9 which shows a nucleotide sequence;

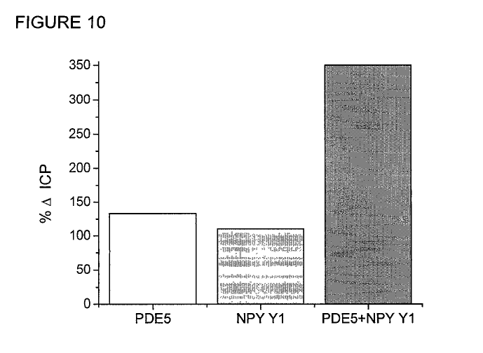

FIG. 10 which shows a graph.

In more detail:

FIG. 1 shows the effect of an NPY Y1 antagonist BIBP3226 (1-100 pg/kg iv) on intracavernosal pressure (ICP) at stimulation in an anaesthetised rabbit. (*P<0.05, Students T-test);

FIG. 2 shows the effect of a PDE 5 inhibitor on intracavernosal pressue (ICP) at stimulation in the anaesthetised rabbit. Data is expressed in percentage increase in ICP over control increases. (*P<0.01, Students T-test unpaired compared with control increases;

FIG. 3 shows the effect of a NPY Y1 antagonist BIBP 3226 (0.03-0.3 mg/kg) on mean arterial blood pressure in the anaesthetised rabbit. Values of mean arterial pressure are expressed as mean±s.e. mean (n=3). Light grey bars represent the basal mean arterial pressure prior to drug administration and the dark grey bars represent the mean arterial pressure following intravenous application of BIBP 3226. White bars represent vehicle controls;

FIG. 4 shows a human neuropeptide Y (NPY) nucleotide sequence (SEQ ID No. 1);

FIG. 5 shows a human NPY Y1 receptor nucleotide sequence (SEQ ID No. 2);

FIG. 6 shows a human NPY Y2 receptor nucleotide sequence (SEQ ID No. 3);

FIGS. 7 and 8 show nucleotide sequences (cDNAs) coding for human SEP (SEQ ID No. 4 and SEQ ID No. 5 respectively). SEQ ID No. 5 includes 5′ and 3′ partial vector sequences;

FIG. 9 shows an amino acid sequence of a human SEP protein (SEQ ID No. 6); and

FIG. 10 shows the effect of an NPY Y1 receptor antagonist, a PDE 5 inhibitor, and a combination of an NPY Y1 receptor antagonist and PDE 5 inhibitor on intracavernosal pressure (ICP) at stimulation in the anaesthetised rabbit. Data is expressed in percentage increase in ICP over control increases.

### EXAMPLES

1.0 Methods

1.1. Animal Test Method

1.1. 1 Anaesthetised Rabbit Methodology

Male New Zealand rabbits (˜2.5 kg) were pre-medicated with a combination of Medetomidine (Domitor®) 0.5 ml/kg i.m., and Ketamine (Vetalar®) 0.25 ml/kg i.m. whilst maintaining oxygen intake via a face mask. The rabbits were tracheotomised using a Portex™ uncuffed endotracheal tube 3 ID., connected to ventilator and maintained at a ventilation rate of 30-40 breaths per minute, with an approximate tidal volume of 18-20 ml, and a maximum airway pressure of 10 cm H₂O. Anaesthesia was then switched to Isoflurane and ventilation continued with O₂ at 2 l/min. The right marginal ear vein was cannulated using a 23G or 24G catheter, and Lactate Ringer solution perfused at 0.5 ml/min. The rabbit was maintained at 3% Isoflurane during invasive surgery, dropping to 2% for maintenance anaesthesia. The left jugular vein was exposed, isolated and then cannulated with a PVC catheter (17G) for the infusion of drugs and compounds.

The left groin area of the rabbit was shaved and a vertical incision was made approximately 5 cm in length along the thigh. The femoral vein and artery were exposed, isolated and then cannulated with a PVC catheter (17G) for the infusion of drugs and compounds. Cannulation was repeated for the femoral artery, inserting the catheter to a depth of 10 cm to ensure that the catheter reached the abdominal aorta. This arterial catheter was linked to a Gould system to record blood pressure. Samples for blood gas analysis were also taken via the arterial catheter. Systolic and diastolic pressures were measured, and the mean arterial pressure calculated using the formula (diastolic x2+systolic)÷3. Heart rate was measured via the pulse oxymeter and Po-ne-mah data acquisition software system (Ponemah Physiology Platform, Gould Instrument Systems Inc).

A ventral midline incision was made into the abdominal cavity. The incision was about 5 cm in length just above the pubis. The fat and muscle was bluntly dissected away to reveal the hypogastric nerve which runs down the body cavity. It was essential to keep close to the side curve of the pubis wall in order to avoid damaging the femoral vein and artery which lie above the pubis. The sciatic and pelvic nerves lie deeper and were located after further dissection on the dorsal side of the rabbit. Once the sciatic nerve is identified, the pelvic nerve was easily located. The term pelvic nerve is loosely applied; anatomy books on the subject fail to identify the nerves in sufficient detail. However, stimulation of the nerve causes an increase in intracavernosal pressure and cavernosal blood flow, and innervation of the pelvic region. The pelvic nerve was freed away from surrounding tissue and a Harvard bipolar stimulating electrode was placed around the nerve. The nerve was slightly lifted to give some tension, then the electrode was secured in position. Approximately 1 ml of light paraffin oil was placed around the nerve and electrode. This acts as a protective lubricant to the nerve and prevents blood contamination of the electrode. The electrode was connected to a Grass S88 Stimulator. The pelvic nerve was stimulated using the following parameters:−5V, pulse width 0.5 ms, duration of stimulus 20 seconds with a frequency of 16 Hz. Reproducible responses were obtained when the nerve was stimulated every 15-20 minutes. Several stimulations using the above parameters were performed to establish a mean control response. The compound(s) to be tested were infused, via the jugular vein, using a Harvard 22 infusion pump allowing a continuous 15 minute stimulation cycle. The skin and connective tissue around the penis was removed to expose the penis. A catheter set (Insyte-W, Becton-Dickinson 20 Gauge 1.1×48 mm) was inserted through the tunica albica into the left corpus cavemosal space and the needle removed, leaving a flexible catheter. This catheter was linked via a pressure transducer (Ohmeda 5299-04) to a Gould systerl to record intracavernosal pressure. Once an intracavernosal pressure was established, the catheter was sealed in place using Vetbond (tissue adhesive, 3M). Heart rate was measured via the pulse oxymeter and Po-ne-mah data acquisition software system (Ponemah Physiology Platform, Gould Instrument Systems Inc).

Intracavernosal blood flow was recorded either as numbers directly from the Flowmeter using Po-ne-mah data acquisition software (Ponemah Physiology Platform, Gould Instrument Systems Inc), or indirectly from Gould chart recorder trace. Calibration was set at the beginning of the experiment (0-125 ml/min/100 g tissue). The NPY inhibitor was made up in saline +10% 1M NaOH, the phosphodiesterase type 5 (PDE5) inhibitor was made up in saline +5% 1 M HCl. The inhibitors and vehicle controls were infused at a rate of 0.1 ml/second. NPY inhibitors and PDE_{cAMP} inhibitors were left for 15 minutes prior to pelvic nerve stimulation.

All data are reported as mean±s.e.m. Significant changes were identified using Student's t-tests.

2.0 Results and Discussion

2.1 NPY Receptor Antagonist

There are a number of anaesthetised animal models of erection which mimic the physiology of penile erection, i.e. increases in penile blood flow and intra cavernosal pressure. The effects of sexual arousal are mimicked by stimulation of pelvic neurones that innervate the penis. This is a mechanism to investigate erectile mechanisms and to assess potential therapeutic agents for the treatment of MED.

It is now established that selective PDE5 inhibitors such as sildenafil enhance nerve stimulated-increases in intracavernosal pressure (ICP) in animal model s and that nerve stimulation mimics the erectile process observed in man (Carter et al., 1998, Traish et al., 1999, Omote 1999, Wallis 1999). This PDE5 inhibitor-induced enhancement of ICP characterises the mechanism of action of PDE5 inhibitors and explains how agents such as sildenafil overcomes any relaxant deficiencies associated with MED or impotence. In agreement with these previous studies, the examples hereinafter have demonstrated that a selective PDE5 inhibitor, administered intravenously, potentiates nerve-stimulated increases in ICP in the anaesthetised rabbit (Example 2).

The examples hereinafter demonstrate that inhibition of NPY receptors with a selective NPY Y1 receptor antagonist (BIBP3226) dose-dependently potentiates nerve stimulated increases in intracavernosal pressure in the anaesthetised rabbit (Example 1). At the doses used in this study a similar enhancement of the erectile process was observed with a NPY antagonist as was observed with a PDE5 inhibitor (Example 2). These examples underline the potential clinical application of a NPY receptor antagonist therapy to enhance the erectile process and hence in the treatment of MED.

Concomitant inhibition of an NPY or NPY Y1 receptor and a PDE5 receptor produced a marked enhancement of the ICP, or the erectile process, than was achievable with the same dose of the same PDE5 inhibitor alone. Using the rabbit model of erection, we can demonstrate that the potentiation of ICP induced by PDE5 inhibition can be further potentiated by co-administration of a NPY Y1 receptor antagonist. At 1 mg/kg

### Example 3

### Effect of Agents that Enhance Intracavernosal Pressure on the Mean Arterial Blood Pressure in the Anaesthetised Rabbit

In the search for novel therapies to treat male sexual dysfunctions such as MED it is desirable that there are no associated adverse cardiovascular effects eg effects on blood pressure or heart rate. In our studies, we have found that a NPY Y1 receptor antagonist BIBP3226 (0.03-0.3 mg/kg) had no substantial effect on blood pressure or heart rate at similar doses to those that enhanced pelvic nerve stimulated increases in intracavernosal pressure.

Intravenous administration of BIBP3226 (a selective NPY Y1 antagonist had no substantial effect the mean arterial blood pressure in the anaesthetised rab it model of penile erection. The graph shown in FIG. 3 demonstrates that BIBP3226 has no significant effect on mean arterial pressure in the anaesthetised rabbit at doses that enhanced pelvic nerve stimulated increases in intracavemosal pressure. Values of mean arterial pressure (MAP) are expressed as mean±s.e.mean (n=3). Light grey bars represent the basal MAP prior to drug administration and dark grey bars represent the MAP following intravenous application of BIBP3226. White bars represent vehicle controls. *P<0.05, Students t-test unpaired compared with control increases.

### Example 4

### NPY 1 Receptor Antagonists Significantly Increases the Efficacy of PDE 5 Inhibitor to Enhance Penile Erection in an Anaesthetised Rabbit Model of Erection.

Intravenous administration of a selective PDE5 inhibitor (1 mg/kg) significantly enhanced nerve-stimulated increases in ICP by 133% compared to contol increases (see Example 2). Intravenous administration of a BIBP 3226 (a selective NPY Y1 antagonist, 100 μg/kg) significantly enhanced nerve-stimulated increases in ICP by 110% compared to control increases. Once the NPY Y1 antagonist-mediated increase was sustained, co-administration of a selective PDE5 inhibitor (1 mg/kg) further enhanced nerve-stimulated increases in ICP to a maximum increase of 350% (see FIG. 10). The degree of potentiation appears to be larger than one would expect with a concomitant application of a NPY Y1 antagonist and a PDE5 inhibitor (ie 133%+110%=243% compared with 350%). Data is expressed as percentage increase in ICP over control increases.

There were no major effects of PDE5 inhibition or combined PDE5 inhibitio/NPY Y1 antagonism on basal/unstimulated intracavernosal pressure.

### Example 5

### NPY Y1 Receptor Antagonists Potentiate the Erectile Effects of PDE 5 Inhibitors and Speeds up the Onset of Action of PDE 5 Inhibitors in Anaesthetised Rabbit Model of Erection.

Early investigations suggest that NPY Y1 receptor antagonists beneficially potentiates the efficacy of a PDE 5 inhibitor and speeds up the onset of PDE 5 inhibitor action in the anaesthetised rabbit model.

### REFERENCES

Allen, J. M.; Bloom, S. R. : Neuropeptide Y: a putative neurotransmitter. Neurochem. Int. 8: 1-8, 1986.

Ammar et al (1996), Genomics Dec 15; 38 (3): 392-8.

Argiolas, A. et al (1995), Neuromodulation of penile erection. Prog Neurobiol. 47: 235-255.

Bahary, N.; Zorich, G.; Pachter, J. E.; Leibel, R. L.; Friedman, J. M. : Molecular genetic linkage maps of mouse chromosomes 4 and 6. Genomics 11: 33-47, 1991.

Baker, E.; Hort, Y. J.; Ball, H.; Sutherland, G. R.; Shine, J.; Herzog, H.: Assignment of the human neuropeptide Y gene to chromosome 7p15.1 by nonisotopic in situ hybridization. Genomics 26: 163-164, 1995.

Benet, A. E. et al (1994), Male erectile dysfunction assessment and treatment options. Comp. Ther. 20: 669-673.

Boolel, M. et al (1996). Sildenafil, a novel effective oral therapy for male erectile dysfunction. Br. J. of Urology 78: 257-261.

Carr, L. G.; Foroud, T.; Bice, P.; Gobbett, T.; Ivashina, J.; Edenberg, H.; Lumeng, L.; Li, T. K.: A quantitative trait locus for alcohol consumption in selectively bred rat lines. Alcohol Clin. Exp. Res. 22: 884-887, 1998.

Carter A J. et al (1998). Effect of the selective phosphodiesterase type 5 inhibitor sildenafil on erectile dysfunction in the anesthetized dog. J. Urol. 160: 242-6.

Chiou, W. F. et al (1998). Relaxation of corpus cavernosum and raised intracavernous pressure by berberine in rabbit. Br. J. Pharmacol. 125: 1677-1684.

Dockray, G. J.: Neuropeptide Y: in search of a function. Neurochem. Int. 8: 9-11, 1986.

Erickson, J. C.; Clegg, K. E.; Palmiter, R. D. : Sensitivity to leptin and susceptibility to seizures of mice lacking neuropeptide Y. Nature 381: 415-421, 1996. PubMed ID: 8632796

Erickson, J. C.; Hollopeter, G.; Palmiter, R. D.: Attenuation of the obesity syndrome of ob/ob mice by the loss of neuropeptide Y. Science 274: 1704-1706, 1996.

Gerald et al 1995, J. Biol Chem, Nov 10; 270(45); 26758-61.

Herzog, H.; Baumgartner, M.; Vivero, C.; Selbie, L. A.; Auer, B.; Shine, J.: Genomic organization, localization, and allelic differences in the gene for the human neuropeptide Y Y1 receptor. J. Biol. Chem. 268: 6703-6707, 1993.

Herzog, H.; Darby, K.; Ball, H.; Hort, Y.; Beck-Sickinger, A.; Shine, J.: Overlapping gene structure of the human neuropeptide Y receptor subtypes Y1 and Y5 suggests coordinate transcriptional regulation. Genomics 41: 315-319, 1997.

Herzog, H.; Hort, Y. J.; Ball, H. J.; Hayes, G.; Shine, J.; Selbie, L. A.: Cloned human neuropeptide Y receptor couples to two different second messenger systems. Proc. Nat. Acad. Sci. 89: 5794-5798,1992.

Jacques et al (1995) European Journal of Pharmacology 278, 1; R3-R5.

Jeremy, J. Y. et al (1997). Effects of sildenafil, a type-5 cGMP phosphodiest rase inhibitor, and papaverine on cyclic GMP and cyclic AMP levels in the rabbit corpus cavernosum in vitro. Br. J. Urology 79: 958-963.

Karvonen, M. K.; Pesonen, U.; Koulu, M.; Niskanen, L.; Laakso, M.; Rissanen, A.; Dekker, J. M.; 't Hart, L. M.; Valve, R.; Uusitupa, M. I.: Association of a leucine(7)-to-proline(7) polymorphism in the signal peptide of neurope tide Y with high serum cholesterol and LDL cholesterol levels. Nature Med. 4: 1434-1437,1998.

Larhammar, D.; Blomqvist, A. G.; Yee, F.; Jazin, E.; Yoo, H.; Wahlestedt, C.: Cloning and functional expression of a human neuropeptide Y/peptide YY receptor of the Y1 type. J. Biol. Chem. 267: 10935-10938, 1992.

Lutz, C. M.; Frankel, W. N.; Richards, J. E.; Thompson, D. A.: Neuropeptide Y receptor genes on human chromosome 4q31-q32 map to conserved linkage groups on mouse chromosomes 3 and 8. Genomics 41: 498-500, 1997.

Lerner, S. E. et al (1993). A review of erectile dysfunction: new insights and more questions. J. Urology 149: 1246-1255.

Maccarrone, C.; Jarrott, B.: Neuropeptide Y: a putative neurotransmitter. Neurochem. Int. 8: 13-22, 1986.

Meisler, M. H.; Spence, J. E.; Dixon, J. E.; Caldwell, R. M.; Minth, C. D.; Beaudet, A. L.: Exclusion of close linkage between the loci for cystic fibrosis and neuropeptide Y on human chromosome 7. Cytogenet. Cell Genet. 44: 175-176, 1987.

Melman, A. & Gingell, J.C. (1999). The epidemiology and pathophysiolog y of erectile dysfunction. J. Urology 161: 5-11.

Minth, C. D.; Andrews, P. C.; Dixon, J. E. : Characterization, sequence, and expression of the cloned human neuropeptide Y gene. J. Biol. Chem. 261: 11974-11979, 1986.

Minth, C. D.; Bloom, S. R.; Polak, J. M.; Dixon, J. E.: Cloning, characterization, and DNA sequence of a human cDNA encoding neuropeptide tyrosine. Proc. Nat. Acad. Sci. 81: 4577-4581, 1984.

Montague, D. et al (1996). Clinical guidelines panel on erectile dysfunction: Summary report on the treatment of organic erectile dysfunction. J. Urology 156: 2007-2011.

Naylor, A. M. (1998). Endogenous neurotransmitters mediating penile erection. Br. J. Urology 81: 424-431.

NIH Consensus Development Panel on Impotence (1993). NIH Consensus Conference Impotence. J.A.M.A. 270: 83.

Omote M. (1999). Pharmacological profiles of sildenafil (VIAGRA) in the treatment of erectile dysfunction: efficacy and drug interaction with nitrate. Nippon Yikurigaku Zasshi. 114:213-8.

Rose et al (1995) J. Biol Chem, Sep 29; 270(39); 22661-4.

Rudolf et at (1994) European Journal of Pharmacology 271, 2-3; R11-R13.

Takeuchi, T.; Gumucio, D.; Eddy, R.; Meisler, M.; Minth, C.; Dixon, J.; Yamada, T.; Shows, T. : Assignment of the related pancreatic polypeptide (PPY) and neuropeptide Y (NPY) genes to regions on human chromosomes 17 and 7. (Abstract) Cytogenet. Cell Genet. 40: 759 only, 1985.

Takeuchi, T.; Gumucio, D. L.; Yamada, T.; Meisler, M. H.; Minth, C. D.; Dixon, J. E.; Eddy, R. E.; Shows, T. B.: Genes encoding pancreatic polypeptide and neuropeptide Y are on human chromosomes 17 and 7. J. Clin. Invest 77:1038-1041, 1986.

Taub, H. C. et al (1993). Relationship between contraction and relaxation in human and rabbit corpus cavernosum. Urology 42: 698-704.

Terenghi, G.; Polak, J. M.; Hamid, Q.; O'Brien, E.; Denny, P.; Legon, S.; Dixon, J.; Minth, C. D.; Palay, S. L.; Yasargil, G.; Chan-Palay, V. : Localization of neuropeptide Y mRNA in neurons of human cerebral cortex by means of in situ hybridization with a complementary RNA probe. Proc. Nat. Acad. Sci. 84: 7315-7318, 1987.

Thiele, T. E.; Marsh, D. J.; Ste. Marie, L.; Bernstein, I. L.; Palmiter, R. D.: Ethanol consumption and resistance are inversely related to neuropeptide Y levels. Nature 396: 366-369, 1998.

Traish A M, et al (1999). Effects of castration and androgen replacement on erectile function in a rabbit model. Endocrinology. 140: 1861-8.

Uusitupa, M. I. J.; Karvonen, M. K.; Pesonen, U.; Koulu, M.: Neuropeptide Y: a novel link between the neuroendocrine system and cholesterol metabolism. Ann. Med. 30: 508-510, 1998.

Abbreviations

| | |
|---|---|
| cAMP = | cyclic adenosine-3′,5′-monophosphate |
| cGMP = | cyclic guanosine-3′,5′-monophosphate |
| PDE = | phosphodiesterase |
| PDE_{cGMP =} | cGMP hydrolysing PDE |
| PDEi = | inhibitor of a PDE (also known as I:PDE) |
| PDE5 = | phosphodiesterase type 5 |
| PDE5i = | inhibitor of PDE5 |
| NPY = | neuropeptide Y |
| NPYi = | inhibitor of NPY |
| NPY Y1 = | neuropeptide Y Y1 receptor |
| NPY Y1i = | inhibitor of NPY Y1 |
| kDa = | kilodalton |
| bp = | base pair |
| kb = | kilobase pair |

1. The use of an inhibitor of a neuropeptide Y (NPY), which inhibitor when in use is selective for an NPY associated with male genitalia, in the preparation of a medicament for the treatment or prevention of male erectile dysfunction (MED).

2. The use of an inhibitor of neuropeptide Y Y1 receptor (NPY Y1), which inhibitor when in use is selective for an NPY Y1 associated with male genitalia, in the preparation of a medicament for the treatment or prevention of MED.

3. The use according to claim 1 or claim 2, wherein said inhibitor when in use is highly selective for NPY/NPY Y1 located in male genitalia.

4. The use according to any one of claims 1-3, wherein said inhibitor has no, or substantially no, activity towards endopeptidase NEP and/or angiotensin converting enzyme.

5. The use according to any one of the preceding claims wherein said treatment or prevention of MED is selective.

6. The use according to any one of the preceding claims wherein an increase in intracavernosal pressure is observed.

7. The use according to any one of the preceding claims wherein the medicament is administered by mouth.

8. The use according to any one of the preceding claims wherein said inhibitor is when in use highly selective for NPY and/or NPY Y1 receptors associated with the corpus cavernosum.

9. The use according to any one of the preceding claims, wherein said NPY and/or NPY Y1inhibitor is administered before and/or during sexual arousal.

10. The use of an NPY Y1 inhibitor in the manufacture of a medicament for selectively increasing the intracavernosal pressure during sexual arousal.

11. A pharmaceutical composition for use in the treatment of male erectile dysfunction (MED); the pharmaceutical composition comprising an inhibitor of a neuropeptide Y (NPY), which inhibitor when in use is selective for an NPY associated with male genitalia; wherein the inhibitor is optionally admixed with a pharmaceutically acceptable carrier, diluent or excipient.

12. A pharmaceutical composition according to claim 10 wherein the inhibitor is an inhibitor of NPY Y1.

13. A method of treating or preventing MED in a human or animal which method comprises administering to an individual an effective amount of an NPYi, which NPYi when in use is selective for an NPY associated with male genitalia, wherein the NPYi, is optionally admixed with a pharmaceutically acceptable carrier, diluent or excipient.

14. A method according to claim 13 wherein the inhibitor is an NPY Y1inhibitor.

15. A method of treating or preventing MED in a human or animal which method comprises delivering to an individual an NPYi that is capable of selectively increasing the intracavernosal pressure during sexual arousal.

16. A method according to claim 15 wherein said NPYi is NPY Y1i.

17. An assay method for identifying an agent that can be used to treat MED, the assay comprising: determining whether a test agent can directly enhance the endogenous erectile process; wherein said enhancement is defined as a potentiation of intracavernosal pressure (and/or cavernosal blood flow) in the presence of a test agent; such potentiation by a test agent is indicative that the test agent may be useful in the treatment of MED and wherein said test agent is an NPYi.

18. An assay according to claim 17 wherein said test agent is an NPY Y1.

19. An assay according to claim 17 or claim 18 wherein said test agent selectively inhibits NPY or NPY Y1 receptors associated with the genitalia.

20. A process comprising the steps of:
(a) performing an assay according to any one of claims 17-19;
(b) identifying one or more agents capable of inhibiting NPY or NPY Y1; and
(c) preparing a quantity of those one or more identified agents; and wherein said agent is a NPYi or an NPY Y1i.

21. A process according to claim 20 wherein said process further comprises testing said one or more agents identified in step (b) for their effect on arterial blood pressure and selecting agents with no, or substantially no, effect on blood pressure.

22. An assay method for identifying an agent that can be used to treat or prevent MED, the assay comprising: contacting a test agent which has a moiety capable of inhibiting the metabolic breakdown of a peptide (preferably a fluorescent labelled peptide), said peptide being normally metabolised by NPY or NPY Y1; and measuring the activity and/or levels of peptide remaining afte r a fixed time (for example via fluorometric analysis); wherein the change in the level of the peptide measured by fluorescence is indicative of the potency (IC50) of the test agent and is indicative that the test agent may be useful in the treatment of MED; and wherein said test agent is an NPYi.

23. An assay according to claim 22 wherein said test agent is an NPY Y1.

24. A method of treating MED with an agent; wherein the agent is capable of inhibiting NPY or NPY Y1in an in vitro assay method; wherein the in vitro assay method is the assay method defined in any one of claims 22-23.

25. An agent identified by the assay methods according to claims 17-19 or claims 22-23.

26. An agent according to claim 25 for use in treating or preventing MED.

27. A medicament for oral administration to treat MED, wherein the medicament comprises the agent according to claim 25.

28. A diagnostic method wherein the method comprises: isolating a sample from a male; determining whether the sample contains an entity present in such an amount as to cause MED; wherein the entity has a direct effect on the endogenous erectile process in the corpus cavernosum of the male; and wherein said entity can be modulated to achieve a beneficial effect by use of an agent, and wherein said agent is an NPYi or an NPY Y1i.

29. A diagnostic composition or kit comprising means for detecting an entity in an isolated male sample; wherein the means can be used to determine whether the sample contains the entity and in such an amount to cause MED, or is in an amount so as to cause MED; wherein the entity has a direct effect on the endogenous erectile process and wherein said entity can be modulated to achieve a beneficial effect by use of an agent; and wherein said agent is an NPYi or an NPY Y1i.

30. An animal model for identifying agent capable of treating MED, said model comprising an anaesthetised animal including means to measure changes in intracavernosal pressure and/or cavemosal blood flow of said animal following stimulation of the pelvic nerve thereof; and wherein said agent is an NPYi or an NPY Y1i.

31. An animal model according to claim 30 wherein said model further comprising means to measure arterial blood pressure of said animal.

32. An assay method for identifying an agent that can directly enhance the endogenous erectile process in order to treat MED, the assay method comprising: administering an agent to the animal model of claim 30 or claim 31; and measuring the change in the endogenous erectile process; wherein said change is defined as a potentiation of intracavernosal pressure (and/or cavernosal blood flow) in the animal model in the presence of a test agent as defined; and wherein said agent is an NPYi or an NPY Y1i.

33. The use according to any one of claims 1-10, wherein in addition to the treatment of MED, abnormal drink and food intake disorders, in particular obesity, anorexia, bulimia and metabolic disorders are also treated.

34. The use of a combination consisting of one or more NPYi's and one of the following auxiliary active agents in the manufacture/preparation of a medicament for the treatment or prevention of MED:
(i) Naturally occurring or synthetic prostaglandins or esters thereof.;
(ii) α-adrenergic receptor antagonist compounds;
(iii) NO-donor (NO-agonist) compounds;
(iv) Potassium channel openers or modulators;
(v) Dopaminergic agents, preferably apomorphine or a selective D2, D3 or D2/D₃agonist;
(vi) Vasodilator agents;
(vii) Thromboxane A2 agonists;
(viii) CNS active agents;
(ix) Ergot alkoloids;
(x) Compounds which modulate the action of natruretic factors in particular atrial naturetic factor (also known as atrial naturetic peptide), B type and C type naturetic factors such as inhibitors or neutral endopeptidase;
(xi) Angiotensin receptor antagonists such as losartan;
(xii) Substrates for NO-synthase, such as L-arginine;
(xiii) Calcium channel blockers such as amlodipine;
(xiv) Antagonists of endothelin receptors and inhibitors or endothelin-converting enzyme;
(xv) Cholesterol lowering agents such as statins (e.g. atorvastatin/Lipitor-trade mark) and fibrates;
(xvi) Antiplatelet and antithrombotic agents, e.g. tPA, uPA, warfarin, hirudin and other thrombin inhibitors, heparin, thromboplastin activating factor inhibitors;
(xvii) Insulin sensitising agents such as rezulin and hypoglycaemic agents such as glipizide;
(xviii) L-DOPA or carbidopa;
(xix) Acetylcholinesterase inhibitors such as donezipil;
(xx) Steroidal or non-steroidal anti-inflammatory agents;
(xxi) Estrogen receptor modulators and/or estrogen agonists a d/or estrogen antagonists;
(xxii) A PDE inhibitor, more particularly a PDE 2, 3, 4, 5, 7 or 8 inhibitor, preferably PDE2 or PDE5 inhibitor and most preferably a PDE5 inhibitor;
(xxiii) An NEP inhibitor;
(xxiv) Vasoactive intestinal protein (VIP), VIP mimetic, VIP analogue, more particularly mediated by one or more of the VIP receptor subtypes VPAC1, VPAC or PACAP (pituitory adenylate cyclase activating peptide), one or more of a VIP receptor agonist or a VIP analogue or a VIP fragment, one or more of a α-adrenoceptor antagonist with VIP combination;
(xxv) A melanocortin receptor agonist or modulator or melanoc ortin enhancer;
(xxvi) A serotonin receptor agonist, antagonist or modulator, more particularly agonists, antagonists or modulators for 5HT1A (including VML 670), 5HT2A, 5HT2C, 5HT3 and/or 5HT6 receptors;
(xxvii) A testosterone replacement agent (including dehydroandrostendione), testosternone (Tostrelle), dihydrotestosterone or a testosterone implant;
(xxviii) Estrogen, estrogen and medroxyprogesterone or medroxyprogesterone acetate (MPA) (i.e. as a combination), or estrogen and methyl testosterone hormone replacement therapy agent;
(xxix) A modulator of transporters for noradrenaline, dopamine and/or serotonin;
(xxx) A purinergic receptor agonist and/or modulator;
(xxxi) A neurokinin (NK) receptor antagonist;
(xxxii) An opioid receptor agonist, antagonist or modulator, preferably agonists for the ORL-1 receptor;
(xxxiii) An agonist or modulator for oxytocin/vasopressin receptors, preferably a selective oxytocin agonist or modulator;
(xxxiv) Modulators of cannabinoid receptors;
(xxxv) A bombesin receptor antagonist, more particularly a bombesin BB₁, BB₂, BB₃, or BB₄ receptor antagonist, preferably a bombesin BB₁ inhibitor;
(xxxvi) A SEP inhibitor;
(xxxvii) An agent capable of modulating the activity of an intermediate conductance calcium-activated potassium (IK_{Ca}) channel in the sexual genitalia of an individual.

35. The use of a combination consisting of one or more NPYi's and one or more PDEi's in the manufacture/preparation of a medicament for the treatment or prevention of MED.

36. The use according to claim 35 wherein said NPYi is an NPY Y1i.

37. The use according to claim 35 or claim 36 wherein said PDEi is a PD E5i.

38. The use according to any one of claims 35-37 wherein the medicament is administered by mouth.

39. A pharmaceutical composition consisting of one or more NPYi's and one or more PDEi's, optionally admixed with a pharmaceutically acceptable carrier, diluent or excipient.

40. A pharmaceutical composition according to claim 39 wherein said NPYi is a NPY Y1i.

41. A pharmaceutical composition according to claims 39 or 40 wherein said NPY Y1i is highly selective for NPY Y1 receptors associated with genitala.

42. A pharmaceutical composition according to claim 39 or claim 41 wherein said PDEi is a PDE5i.

43. A pharmaceutical composition according to any one of claims 39 to 42 wherein the composition is administered by mouth.

44. The use of a pharmaceutical composition according to any one of claims 39-43 in the preparation of a medicament for the treatment or prevention of MED.
